(19) United States
(12) Patent Application Publication
(10) Pub. No.: US 2021/0210684 A1
(43) Pub. Date: Jul. 8, 2021

(54) PLURALITY OF ORGANIC ELECTROLUMINESCENT MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Dong Hyung Lee, Gyeonggi-do (KR); Chi-Sik Kim, Gyeonggi-do (KR); Soo-Yong Lee, Gyeonggi-do (KR); Seung-Hoon Yoo, Gyeonggi-do (KR); Doo-Hyeon Moon, Gyeonggi-do (KR); Hyun-Woo Kang, Gyeonggi-do (KR)

(21) Appl. No.: 17/119,671

(22) Filed: Dec. 11, 2020

(30) Foreign Application Priority Data
Jan. 3, 2020 (KR) 10-2020-0000822

(51) Int. Cl. H01L 51/00; C09K 11/02; C09K 11/06; C07C 211/61

(52) U.S. Cl. CPC H01L 51/006; C09K 11/02; C09K 11/06; H01L 51/506; C09K 2211/1007; C09K 2211/1011; C09K 2211/1014; C07C 211/61

(57) ABSTRACT

The present disclosure relates to a plurality of organic electroluminescent materials comprising at least one first compound and at least one second compound, and an organic electroluminescent device comprising the same. By comprising a specific combination of compounds, in which the first compound is represented by formula 1 and the second compound is represented by formula 2, it is possible to provide an organic electroluminescent device having improved lifetime properties,

### TECHNICAL FIELD

The present disclosure relates to a plurality of organic electroluminescent materials and an organic electroluminescent device comprising the same.

### BACKGROUND ART

An electroluminescent device (EL device) is a self-light-emitting display device which has advantages in that it provides a wider viewing angle, a greater contrast ratio, and a faster response time. The first organic electroluminescent device was developed by Eastman Kodak in 1987, by using small aromatic diamine molecules and aluminum complexes as materials for forming a light-emitting layer (see Appl. Phys. Lett. 51, 913, 1987).

An organic electroluminescent device (OLED) changes electric energy into light by applying electricity to an organic electroluminescent material, and commonly comprises an anode, a cathode, and an organic layer formed between the two electrodes. The organic layer of the OLED may comprise a hole injection layer, a hole transport layer, an electron blocking layer, a light-emitting layer, an electron buffer layer, a hole blocking layer, an electron transport layer, an electron injection layer, etc. The materials used in the organic layer can be classified into a hole injection material, a hole transport material, an electron blocking material, a light-emitting material, an electron buffer material, a hole blocking material, an electron transport material, an electron injection material, etc., depending on their functions. In the OLED, holes from the anode and electrons from the cathode are injected into a light-emitting layer by the application of electric voltage, and excitons having high energy are produced by the recombination of the holes and electrons. The organic light-emitting compound moves into an excited state by the energy and emits light from energy when the organic light-emitting compound returns to the ground state.

The selection of a compound included in a hole transport layer or the like has been recognized as a means to improve the properties of a device such as hole transport efficiency to the light-emitting layer, luminous efficiency, and lifetime. In addition, the most important factor determining luminous efficiency in an OLED is light-emitting materials. A light-emitting material must have high quantum efficiency, and high electron and hole mobility, and the formed light-emitting layer must be uniform and stable. Light-emitting materials may be categorized into blue, green, and red light-emitting materials dependent on the light-emitting color, and may further include yellow or orange light-emitting materials. The light-emitting material may be used by mixing a host and a dopant in order to improve color purity, luminous efficiency, and stability. In general, a device having excellent EL properties is a structure comprising a light-emitting layer made by doping a dopant on a host, When using such a dopant/host material system, the host material has a great influence on the efficiency and lifetime of the organic electroluminescent device, so the selection thereof is important.

U.S. Patent Application Publication No. 2017/0025609 A1 (published on Jan. 26, 2017) discloses a phenanthrene substituted with an arylamine. In addition, Korean Patent Appln. Laid-Open No. 2017-0130434 A (published on Nov. 28, 2017) discloses an organic electroluminescent device comprising an anthracene derivative compound and a boron derivative compound. However, the aforementioned references do not specifically disclose that the performance of an organic electroluminescent device can be improved by combining a phenanthrene derivative compound and a boron derivative compound.

### DISCLOSURE OF INVENTION

### Technical Problem

The objective of the present disclosure is to provide an organic electroluminescent device having a longer lifetime property compared to a conventional organic electroluminescent device by comprising a specific combination of compounds in an organic layer of an organic electroluminescent device.

### Solution to Problem

As a result of intensive studies to solve the technical problem above, the present inventors found that the above objective can be achieved by a plurality of organic electroluminescent materials comprising at least one first compound and at least one second compound, wherein the first compound is represented by the following formula 1, and the second compound is represented by the following formula 2.

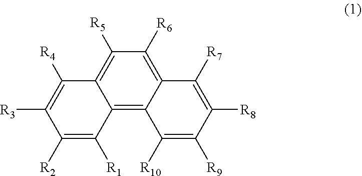

In formula

R₁ to R₁₀, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)alysilyl, or

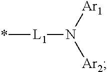

with a proviso that at least one of R₁ to R₁₀ is

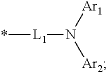

L₁ represents a single bond, a substituted or unsubstituted (C6-C30)arylene, or a substituted or unsubstituted (3- to 30-membered)heteroarylene,

Ar₁ and Ar₂, each independently, represent hydrogen, deuterium, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl, or Ar₁ and Ar₂ may be linked to each other to form a fused ring; and

* represents the position linked to phenanthrene.

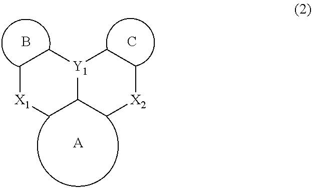

In formula 2,

ring A, ring B, and ring C, each independently, represent a substituted or unsubstituted mono- or polycyclic (3- to 50-membered) alicyclic or aromatic ring, or the combination thereof, which may contain at least one heteroatom selected from B, N, O, S, Si, and P; and ring B and ring C may be linked to each other to form a fused ring;

Y₁ represents B;

X₁ and X₂, each independently, represent NR or O; and

R, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (3- to 30-membered)heteroaryl(C6-C30)arylamino; or may be linked to at least one of ring A, ring B, and ring C to form a fused ring.

### Advantageous Effects of Invention

By comprising a specific combination of compounds according to the present disclosure in an organic layer, it is possible to provide an organic electroluminescent device having longer lifetime and/or higher luminous efficiency properties compared to conventional organic electroluminescent devices, and to manufacture a display system or a lighting system using the same. In addition, with this effect or alternatively, an organic electroluminescent device emitting deeper blue light than conventional organic electroluminescent devices can be provided. Mode for the invention

Hereinafter, the present disclosure will be described in detail. However, the following description is intended to explain the present disclosure, and is not meant in any way to restrict the scope of the present disclosure.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means an organic electroluminescent material(s) comprising a combination of at least two compounds, which may be comprised in any layer constituting an organic electroluminescent device, It may mean both a material before being comprised in an organic electroluminescent device (for example, before vapor deposition) and a material after being comprised in an organic electroluminescent device (for example, after vapor deposition). Specifically, a plurality of organic electroluminescent materials may be a combination of at least two compounds which may be comprised in at least one of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. As an example, a plurality of organic electroluminescent materials of the present disclosure may be a combination of at least one dopant material and at least one hole transport material, and may selectively further comprise a conventional material included in the organic electroluminescent materials. At least two compounds may be comprised in the same layer or different layers through methods used in the art, and may be mixture-evaporated or co-evaporated, or may be individually evaporated.

Hereinafter, the organic electroluminescent device of the present disclosure will be described in more detail.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The alkyl may include methyl, ethyl, n-propyl, iso-propyl, n-butyl, iso-butyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7, preferably 5 to 7, ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms. The above aryl(ene) may be partially saturated, and may comprise a Spiro structure. The number of ring backbone carbon atoms is preferably 6 to 25, and more preferably 6 to 18. The aryl may include phenyl, biphenyl, terphenyl, naphthyl, binaphthyl, phenylnaphthyl, naphthylphenyl, phenylterphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, etc. Specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzofluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, etc.

The term “(3- to 30-membered)heteroaryl(ene)” is meant to be an aryl(ene) having 3 to 30 ring backbone atoms and including at least one, preferably 1 to 4 heteroatoms selected from the group consisting of B, N, O, S, Si, and P. The heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a Spiro structure. The heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. “Halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

In addition, “substituted” in the expression “substituted or unsubstituted” means that a hydrogen atom in a certain functional group is replaced with another atom or another functional group, a substituent. In the present disclosure, the substituents of the substituted alkyl, the substituted aryl, the substituted arylene, the substituted heteroaryl, the substituted heteroarylene, the substituted cycloalkyl, the substituted heterocycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-alkenylamino, the substituted mono- or di-arylamino, the substituted mono- or di-heteroarylamino, the substituted alkylalkenylamino, the substituted alkylarylamino, the substituted alkylheteroarylamino, the substituted alkenylarylamino, the substituted alkenylheteroarylamino, the substituted heteroarylarylamino, and the substituted ring, each independently, are at least one selected from the group consisting of deuterium; a halogen; a cyano; a carboxyl; a nitro; a hydroxyl; a (C1-C30)alkyl unsubstituted or substituted with at least one deuterium; a halo(C1-C30)alkyl, a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium(s), a (C1-C30)alkyl(s), a (C6-C30)aryl(s), and a di(C6-C30)arylamino(s); a (C6-C30)aryl unsubstituted or substituted with at least one of deuterium(s), a (C1-C30)alkyl(s), a (3- to 30-membered)heteroaryl(s), and a di(C6-C30)arylamino(s); a tri(C1-C30)alkylsilyl; a tri(C6-C30)arylsilyl; a di(C1-C30)alkyl(C6-C30)arylsilyl, a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino unsubstituted or substituted with at least one of deuterium(s), a (C1-C30)alkyl(s), a (3- to 30-membered)heteroaryl(s), and a di(C6-C30)arylamino(s); a mono- or di-(3- to 30-membered)heteroarylamino; a (C1-C30)alkyl(C2-C30)alkenylamino; a (C1-C30)alkyl(C6-C30)arylamino, a (C1-C30)alkyl(3- to 30-membered)heteroarylamino; a (C2-C30)alkenyl(C6-C30)arylamino; a (C2-C30)alkenyl(3- to 30-membered)heteroarylamino; a (3- to 30-membered)heteroaryl(C6-C30)arylamino unsubstituted or substituted with at least one of a (C1-C30)alkyl(s) and a (C6-C30)aryl(s); a (C1-C30)alkylcarbonyl, a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl, and a (C1-C30)alkyl(C6-C30)aryl. According to one embodiment of the present disclosure, the substituents, each independently, are at least one selected from the group consisting of deuterium; a (C1-C20)alkyl unsubstituted or substituted with at least one deuterium; a (5- to 25-membered)heteroaryl unsubstituted or substituted with at least one of deuterium(s), a (C1-C20)alkyl(s), and a (C6-C18)aryl(s), a (C6-C25)aryl unsubstituted or substituted with at least one of deuterium(s), a (C1-C20)alkyl(s), a (5- to 25-membered)heteroaryl(s), and a di(C6-C18)arylamino(s); a mono- or di-(C6-C25)arylamino unsubstituted or substituted with at least one of deuterium(s), a (C1-C20)alkyl(s), and a di(C6-C18)arylamino(s); and a (5- to 25-membered)heteroaryl(C6-C18)arylamino unsubstituted or substituted with at least one of a (C1-C20)alkyl(s) and a (C6-C18)aryl(s). According to another embodiment of the present disclosure, the substituents, each independently, are at least one selected from the group consisting of deuterium; a (C1-C10)alkyl unsubstituted or substituted with at least one deuterium; a (5- to 25-membered)heteroaryl unsubstituted or substituted with at least one of deuterium(s) and a (C1-C10)alkyl(s); a (C6-C18)aryl unsubstituted or substituted with at least one of deuterium(s), a (C1-C10)alkyl(s), a (5- to 20-membered)heteroaryl(s), and a di(C6-C18)arylamino(s); a di(C6-C18)arylamino unsubstituted or substituted with at least one of deuterium(s), a (C1-C10)alkyl(s), and a di(C6-C18)arylamino(s); and a (5- to 20-membered)heteroaryl(C6-C18)arylamino unsubstituted or substituted with at least one of a (C1-C10)alkyl(s) and a (C6-C18)aryl(s). For example, the substituents, each independently, may be at least one selected from the group consisting of deuterium; a methyl unsubstituted or substituted with at least one deuterium; a teat-butyl; a phenyl unsubstituted or substituted with at least one of deuterium(s), a methyl(s), a carbazolyl(s), a dibenzofuranyl(s), a phenoxazinyl(s), a phenothiazinyl(s), a 9,9-dimethyl-dihydroacridinyl(s), and a diphenylamino(s); a naphthyl; a biphenyl; a terphenyl; a triphenylenyl; a carbazolyl; a phenoxazinyl; a phenothiazinyl; a 9,9-dimethyl-dihydroacridinyl; a diphenylamino unsubstituted or substituted with at least one of deuterium(s), a methyl(s), a tert-butyl(s); and a diphenylamino(s); a dinaphthylamino; a phenylnaphthylamino; a phenylnaphthylamino substituted with a tert-butyl(s); a dibiphenylarnino; a carbazolylphenylamino substituted with a phenyl(s); a dibenzofuranylphenylamino; and a tetrahydroacridinylphenylamino substituted with at least one methyl.

In the formulas of the present disclosure, when adjacent substituents are linked to each other to form a ring, the ring may be a substituted or unsubstituted mono- or polycyclic (3- to 50-membered) alicyclic or aromatic ring, or the combination thereof; preferably, a substituted or unsubstituted mono- or polycyclic (5- to 40-membered) alicyclic or aromatic ring, or the combination thereof; and more preferably, an unsubstituted mono- or polycyclic (5- to 35-membered) aromatic ring. In addition, the formed ring may contain at least one heteroatom selected from N, O, and S. Further, the ring may be a Spiro ring formed with the backbone structure. For example, the ring may be a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, a substituted or unsubstituted carbazole ring, a substituted or unsubstituted benzoxazine ring, a substituted or unsubstituted benzothiazine ring, a substituted or unsubstituted dihydroquinoline ring, etc.

In the formulas of the present disclosure, the heteroaryl(ene), and the heterocycloalkyl, each independently, may contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

Hereinafter, the compounds represented by formula 1 or 2 will be explained in more detail.

In formula 1, R₁ to R₁₀, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or

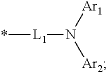

with a proviso that at least one of R₁ to R₁₀ is

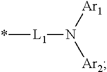

and * represents the position linked to phenanthrene. According to one embodiment of the present disclosure, R₁ to R₁₀, each independently, represent hydrogen, deuterium, a substituted or unsubstituted (C1-C10)alkyl, a substituted or unsubstituted (C6-C25)aryl, a substituted or unsubstituted (5- to 25-membered)heteroaryl, or

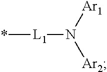

with a proviso that at least one of R₁ to R₁₀ is

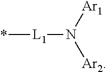

According to another embodiment of the present disclosure, R₁ to R₁₀, each independently, represent hydrogen, deuterium, or

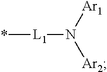

with a proviso that any one of R₁ to R₁₀ is

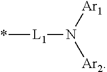

For example, any one of R₁ to R₁₀ may be

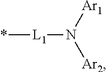

and the others, each independently, may be hydrogen or deuterium.

L₁ represents a single bond, a substituted or unsubstituted (C6-C30)arylene, or a substituted or unsubstituted (3- to 30-membered)heteroarylene. According to one embodiment of the present disclosure, L₁ represents a single bond, or a substituted or unsubstituted (C6-C25)arylene. According to another embodiment of the present disclosure, L₁ represents a single bond, or an unsubstituted (C6-C18)arylene. For example, L₁ may be a single bond, a phenylene, or a naphthylene.

Ar₁ and Ar₂, each independently, represent hydrogen, deuterium, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl, or Ar₁ and Ar₂ may be linked to each other to form a fused ring. Ar₁ and Ar₂ are the same as or different from each other. According to one embodiment of the present disclosure, Ar₁ and Ar₂, each independently, represent a substituted or unsubstituted (C6-C25)aryl, or a substituted or unsubstituted (5- to 25-membered)heteroaryl. According to another embodiment of the present disclosure, Ar₁ and Ar₂, each independently, represent a (C6-C25)aryl unsubstituted or substituted with a (C1-C10)alkyl(s), or a (5- to 25-membered)heteroaryl unsubstituted or substituted with a (C6-C18)aryl(s). For example, Ar₁ and Ar₂, each independently, may be a phenyl, a biphenyl, a naphthylphenyl, a phenylnaphthyl, a terphenyl, a dimethylfluorenyl, a diphenylfluorenyl, 9,9′-spirobifluorenyl, a dibenzothiophenyl, a dibenzofuranyl, or a carbazolyl substituted with a phenyl(s).

The formula 1 may be represented by the following formula 1-1.

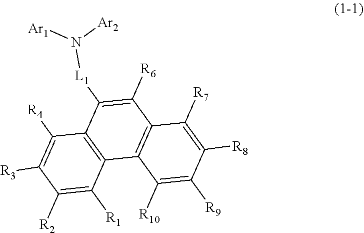

In formula 1-1, L₁, A₁, Ar₂, R₁ to R₄, and R₆ to R₁₀ are as defined in formula 1. The compound represented by formula 1 may be at least one selected from the group consisting of the following compounds, but is not limited thereto.

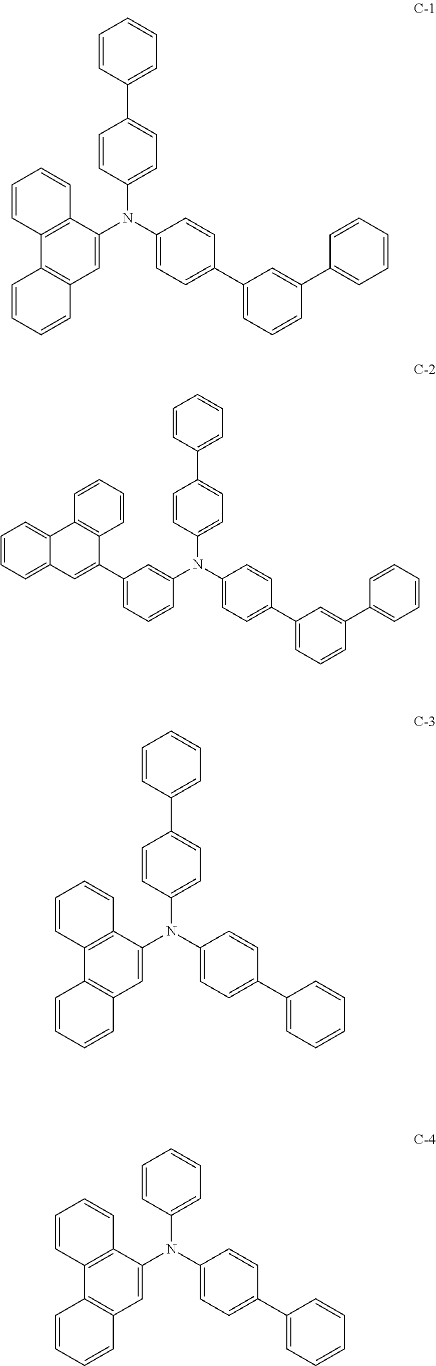 [SMILES: c1ccc(-c2ccc(N(c3ccc(-c4cccc(-c5ccccc5)c4)cc3)c3cc4ccccc4c4ccccc34)cc2)cc1 ; c1ccc(-c2ccc(N(c3ccc(-c4cccc(-c5ccccc5)c4)cc3)c3cccc(-c4cc5ccccc5c5ccccc45)c3)cc2)cc1 ; c1ccc(-c2ccc(N(c3ccc(-c4ccccc4)cc3)c3cc4ccccc4c4ccccc34)cc2)cc1 ; c1ccc(-c2ccc(N(c3ccccc3)c3cc4ccccc4c4ccccc34)cc2)cc1]

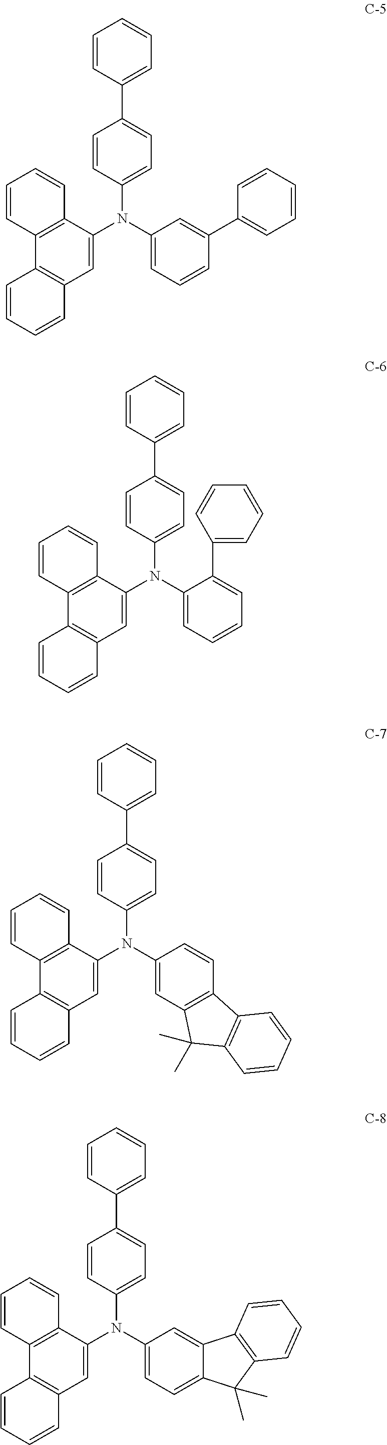 [SMILES: c1ccc(-c2ccc(N(c3cccc(-c4ccccc4)c3)c3cc4ccccc4c4ccccc34)cc2)cc1 ; c1ccc(-c2ccc(N(c3ccccc3-c3ccccc3)c3cc4ccccc4c4ccccc34)cc2)cc1 ; CC1(C)c2ccccc2-c2ccc(N(c3ccc(-c4ccccc4)cc3)c3cc4ccccc4c4ccccc34)cc21 ; CC1(C)c2ccccc2-c2cc(N(c3ccc(-c4ccccc4)cc3)c3cc4ccccc4c4ccccc34)ccc21]

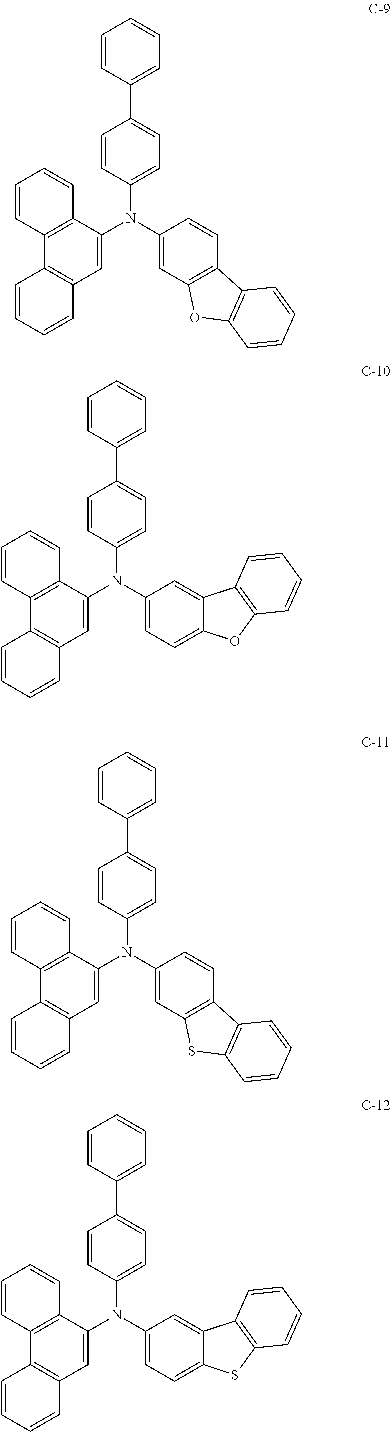 [SMILES: c1ccc(-c2ccc(N(c3ccc4c(c3)oc3ccccc34)c3cc4ccccc4c4ccccc34)cc2)cc1 ; c1ccc(-c2ccc(N(c3ccc4oc5ccccc5c4c3)c3cc4ccccc4c4ccccc34)cc2)cc1 ; c1ccc(-c2ccc(N(c3ccc4c(c3)sc3ccccc34)c3cc4ccccc4c4ccccc34)cc2)cc1 ; c1ccc(-c2ccc(N(c3ccc4sc5ccccc5c4c3)c3cc4ccccc4c4ccccc34)cc2)cc1]

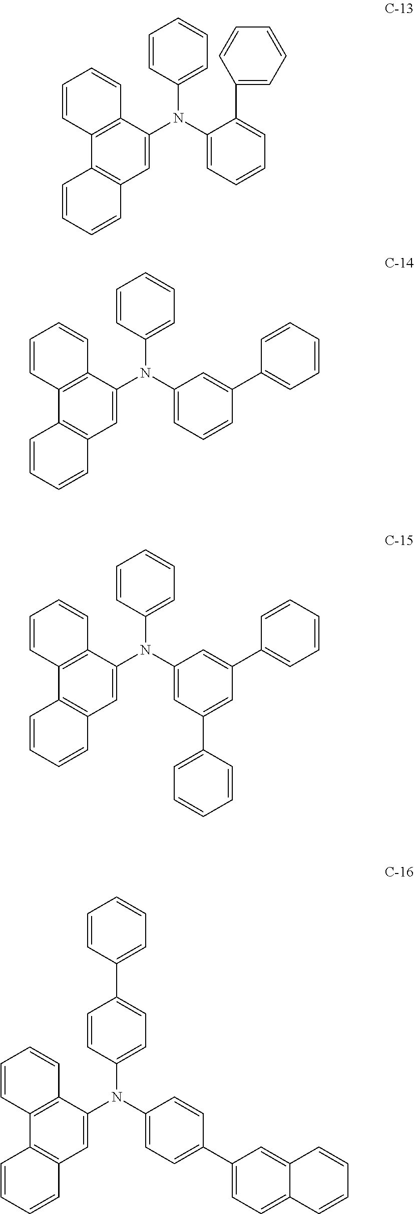 [SMILES: c1ccc(-c2ccccc2N(c2ccccc2)c2cc3ccccc3c3ccccc23)cc1 ; c1ccc(-c2cccc(N(c3ccccc3)c3cc4ccccc4c4ccccc34)c2)cc1 ; c1ccc(-c2cc(-c3ccccc3)cc(N(c3ccccc3)c3cc4ccccc4c4ccccc34)c2)cc1 ; c1ccc(-c2ccc(N(c3ccc(-c4ccc5ccccc5c4)cc3)c3cc4ccccc4c4ccccc34)cc2)cc1]

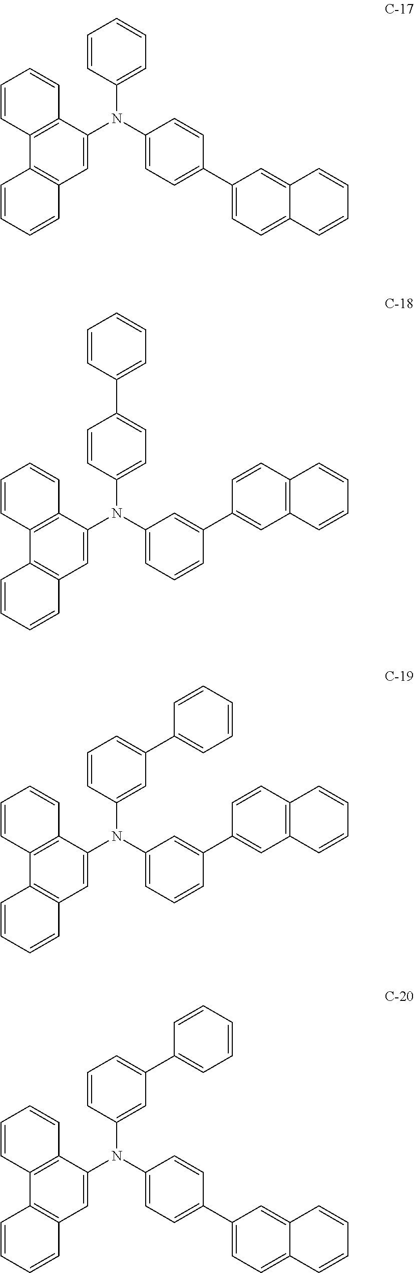 [SMILES: c1ccc(N(c2ccc(-c3ccc4ccccc4c3)cc2)c2cc3ccccc3c3ccccc23)cc1 ; c1ccc(-c2ccc(N(c3cccc(-c4ccc5ccccc5c4)c3)c3cc4ccccc4c4ccccc34)cc2)cc1 ; c1ccc(-c2cccc(N(c3cccc(-c4ccc5ccccc5c4)c3)c3cc4ccccc4c4ccccc34)c2)cc1 ; c1ccc(-c2cccc(N(c3ccc(-c4ccc5ccccc5c4)cc3)c3cc4ccccc4c4ccccc34)c2)cc1]

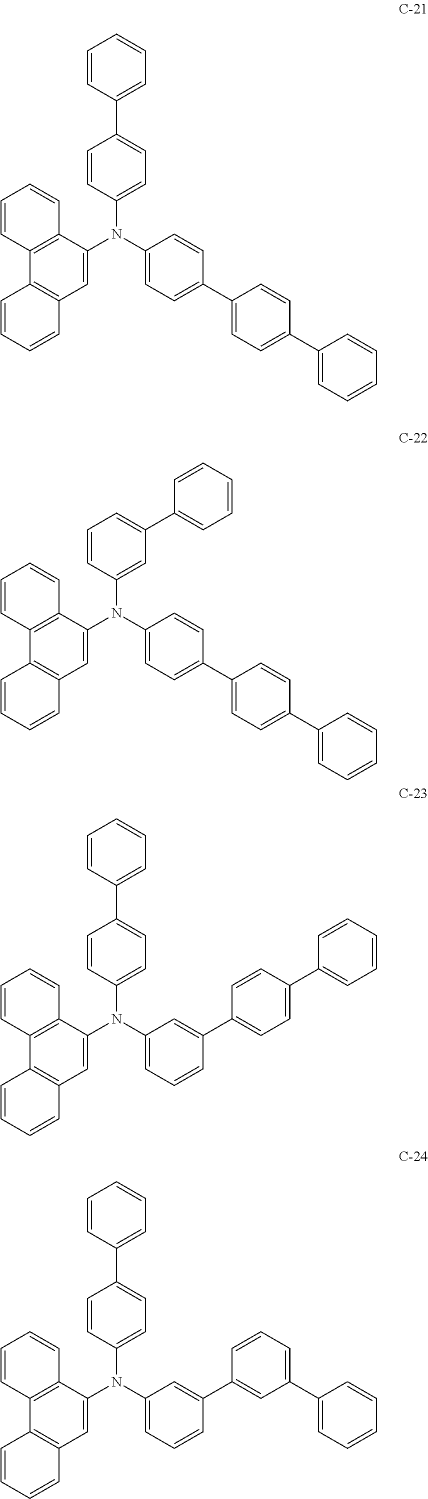 [SMILES: c1ccc(-c2ccc(-c3ccc(N(c4ccc(-c5ccccc5)cc4)c4cc5ccccc5c5ccccc45)cc3)cc2)cc1 ; c1ccc(-c2ccc(-c3ccc(N(c4cccc(-c5ccccc5)c4)c4cc5ccccc5c5ccccc45)cc3)cc2)cc1 ; c1ccc(-c2ccc(-c3cccc(N(c4ccc(-c5ccccc5)cc4)c4cc5ccccc5c5ccccc45)c3)cc2)cc1 ; c1ccc(-c2ccc(N(c3cccc(-c4cccc(-c5ccccc5)c4)c3)c3cc4ccccc4c4ccccc34)cc2)cc1]

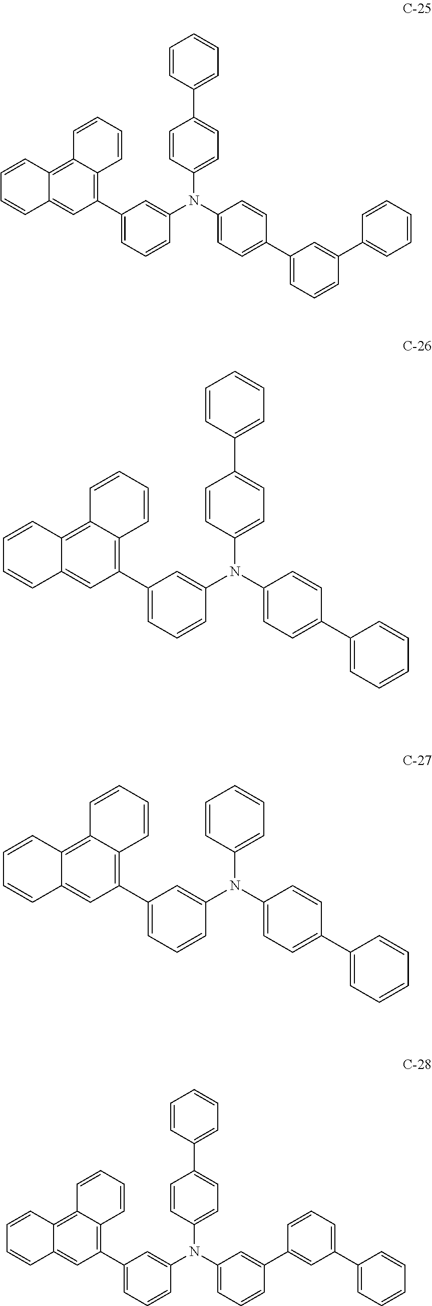 [SMILES: c1ccc(-c2ccc(N(c3ccc(-c4cccc(-c5ccccc5)c4)cc3)c3cccc(-c4cc5ccccc5c5ccccc45)c3)cc2)cc1 ; c1ccc(-c2ccc(N(c3ccc(-c4ccccc4)cc3)c3cccc(-c4cc5ccccc5c5ccccc45)c3)cc2)cc1 ; c1ccc(-c2ccc(N(c3ccccc3)c3cccc(-c4cc5ccccc5c5ccccc45)c3)cc2)cc1 ; c1ccc(-c2ccc(N(c3cccc(-c4cccc(-c5ccccc5)c4)c3)c3cccc(-c4cc5ccccc5c5ccccc45)c3)cc2)cc1]

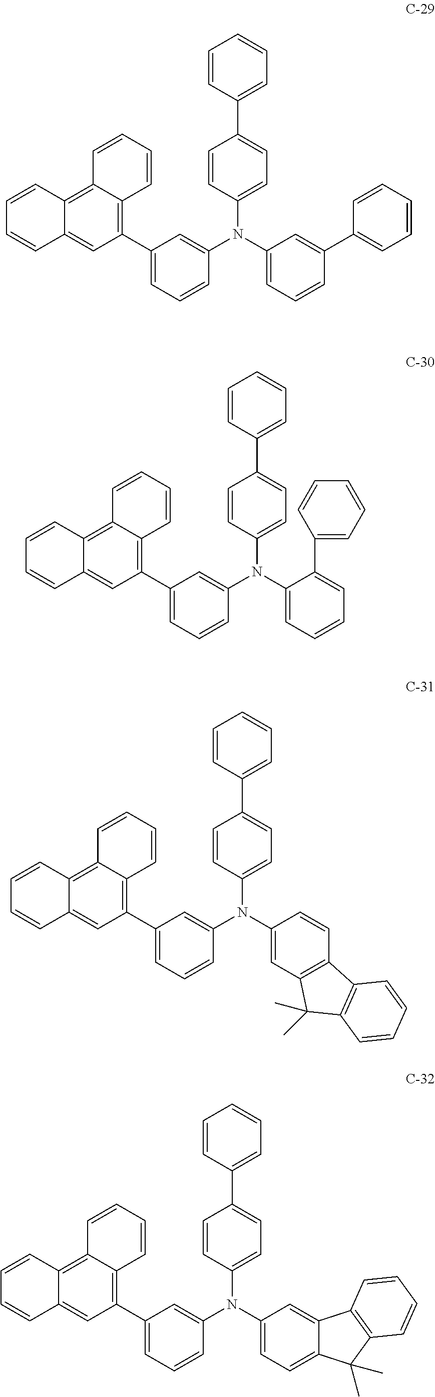 [SMILES: c1ccc(-c2ccc(N(c3cccc(-c4ccccc4)c3)c3cccc(-c4cc5ccccc5c5ccccc45)c3)cc2)cc1 ; c1ccc(-c2ccc(N(c3cccc(-c4cc5ccccc5c5ccccc45)c3)c3ccccc3-c3ccccc3)cc2)cc1 ; CC1(C)c2ccccc2-c2ccc(N(c3ccc(-c4ccccc4)cc3)c3cccc(-c4cc5ccccc5c5ccccc45)c3)cc21 ; CC1(C)c2ccccc2-c2cc(N(c3ccc(-c4ccccc4)cc3)c3cccc(-c4cc5ccccc5c5ccccc45)c3)ccc21]

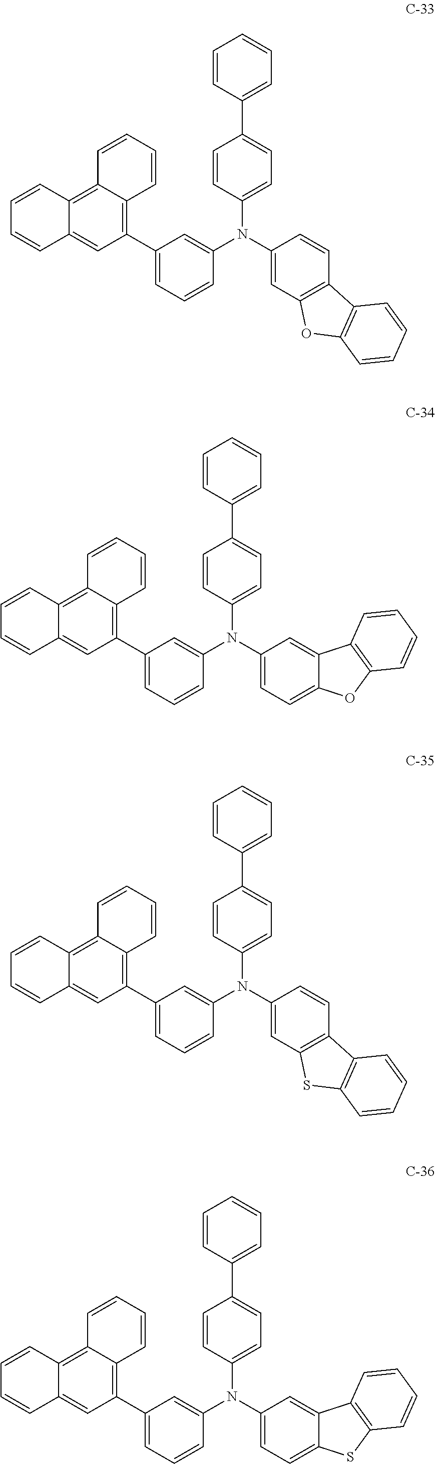 [SMILES: c1ccc(-c2ccc(N(c3cccc(-c4cc5ccccc5c5ccccc45)c3)c3ccc4c(c3)oc3ccccc34)cc2)cc1 ; c1ccc(-c2ccc(N(c3cccc(-c4cc5ccccc5c5ccccc45)c3)c3ccc4oc5ccccc5c4c3)cc2)cc1 ; c1ccc(-c2ccc(N(c3cccc(-c4cc5ccccc5c5ccccc45)c3)c3ccc4c(c3)sc3ccccc34)cc2)cc1 ; c1ccc(-c2ccc(N(c3cccc(-c4cc5ccccc5c5ccccc45)c3)c3ccc4sc5ccccc5c4c3)cc2)cc1]

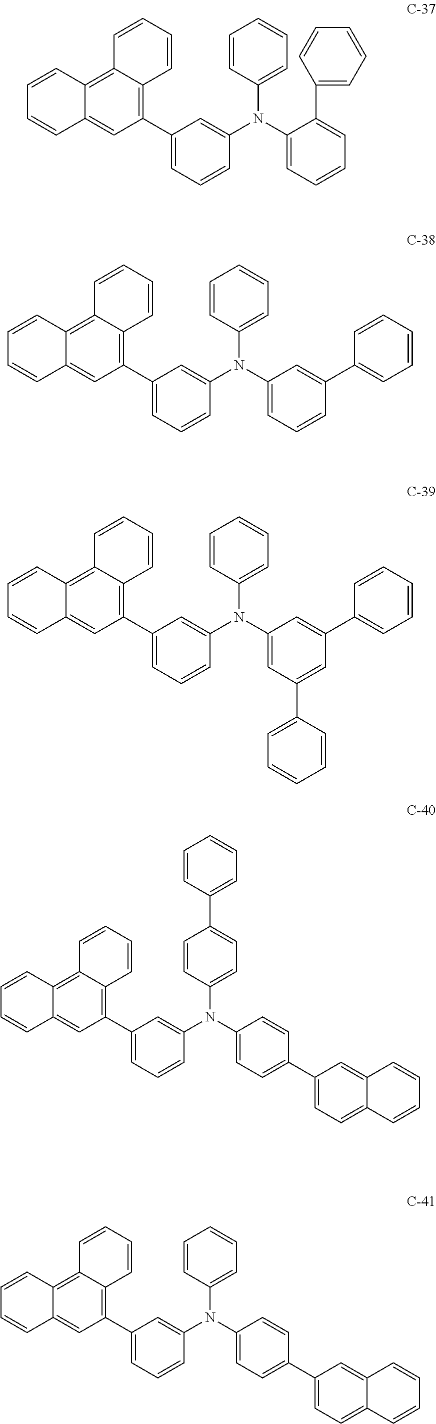 [SMILES: c1ccc(-c2ccccc2N(c2ccccc2)c2cccc(-c3cc4ccccc4c4ccccc34)c2)cc1 ; c1ccc(-c2cccc(N(c3ccccc3)c3cccc(-c4cc5ccccc5c5ccccc45)c3)c2)cc1 ; c1ccc(-c2cc(-c3ccccc3)cc(N(c3ccccc3)c3cccc(-c4cc5ccccc5c5ccccc45)c3)c2)cc1 ; c1ccc(-c2ccc(N(c3ccc(-c4ccc5ccccc5c4)cc3)c3cccc(-c4cc5ccccc5c5ccccc45)c3)cc2)cc1 ; c1ccc(N(c2ccc(-c3ccc4ccccc4c3)cc2)c2cccc(-c3cc4ccccc4c4ccccc34)c2)cc1]

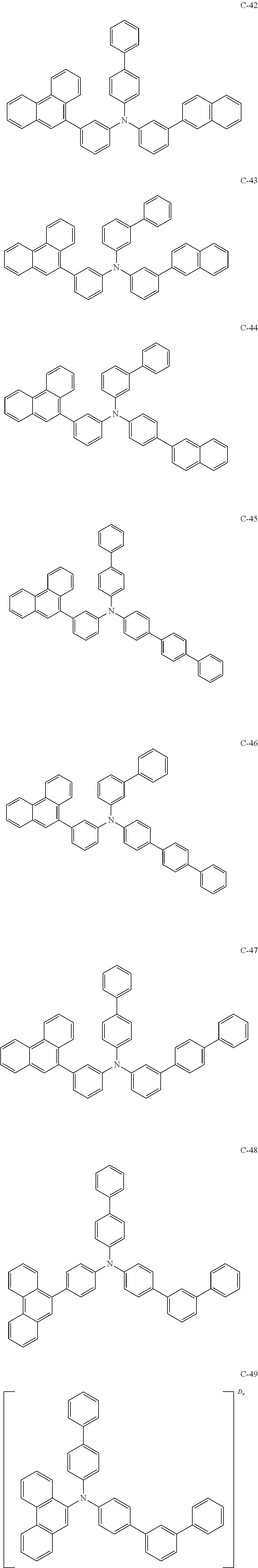

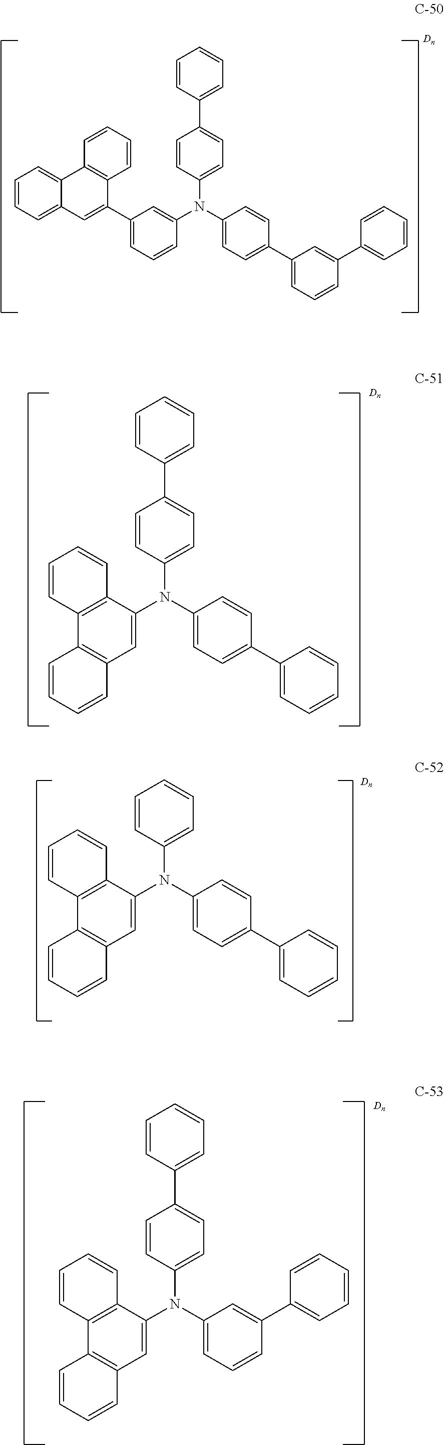

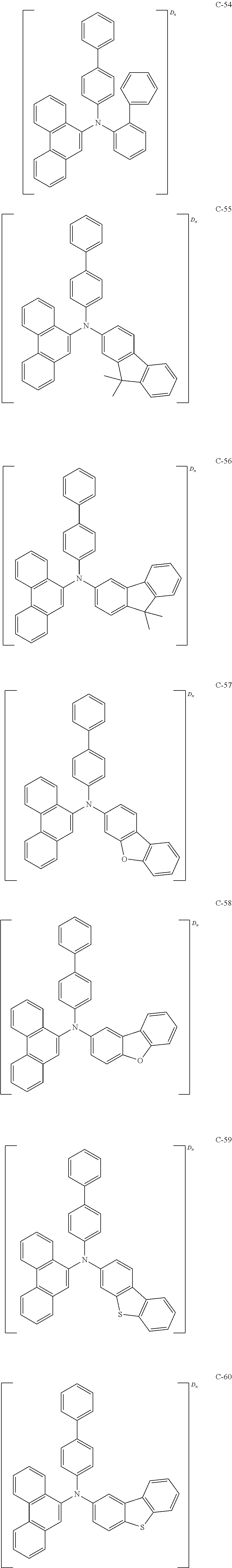

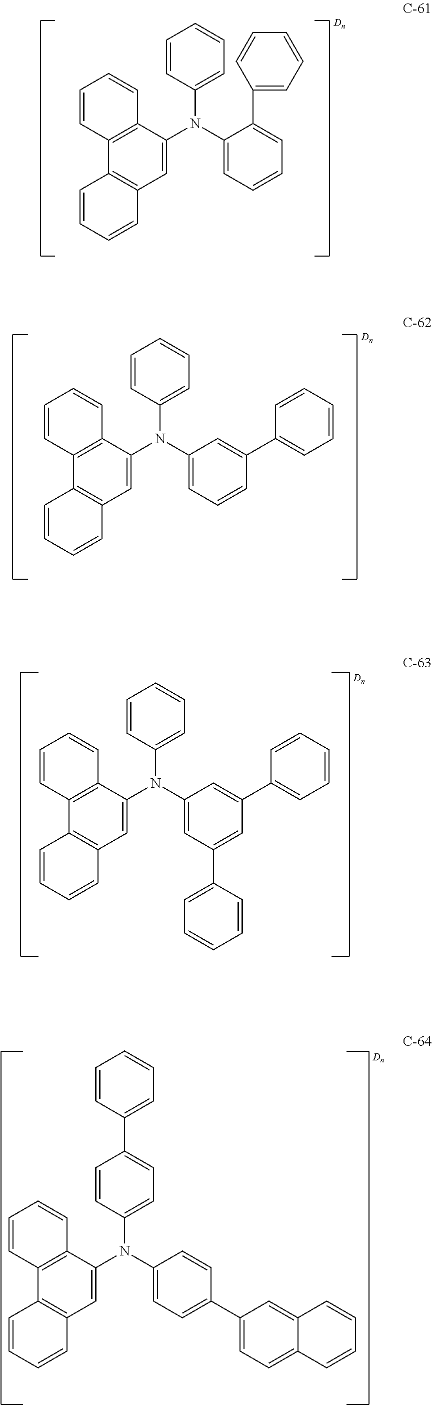

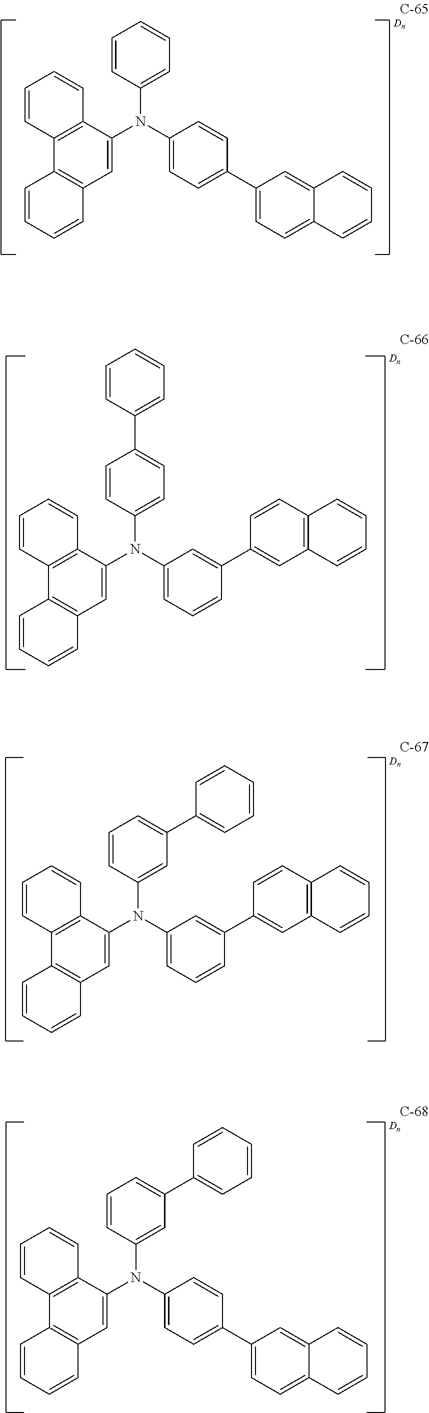

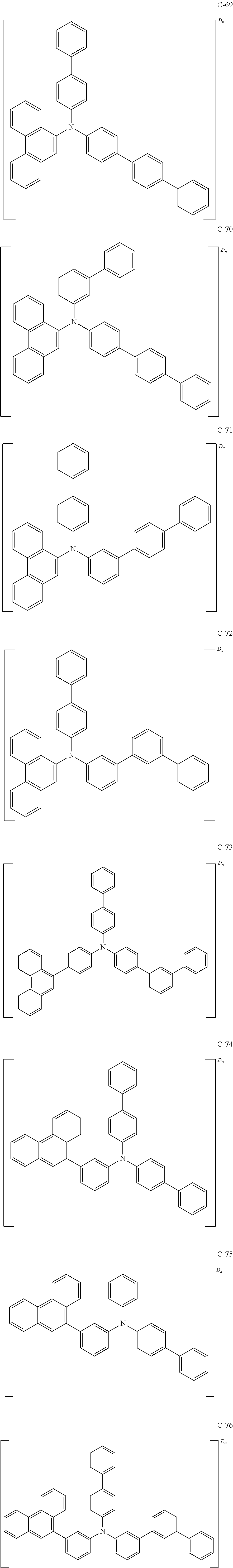

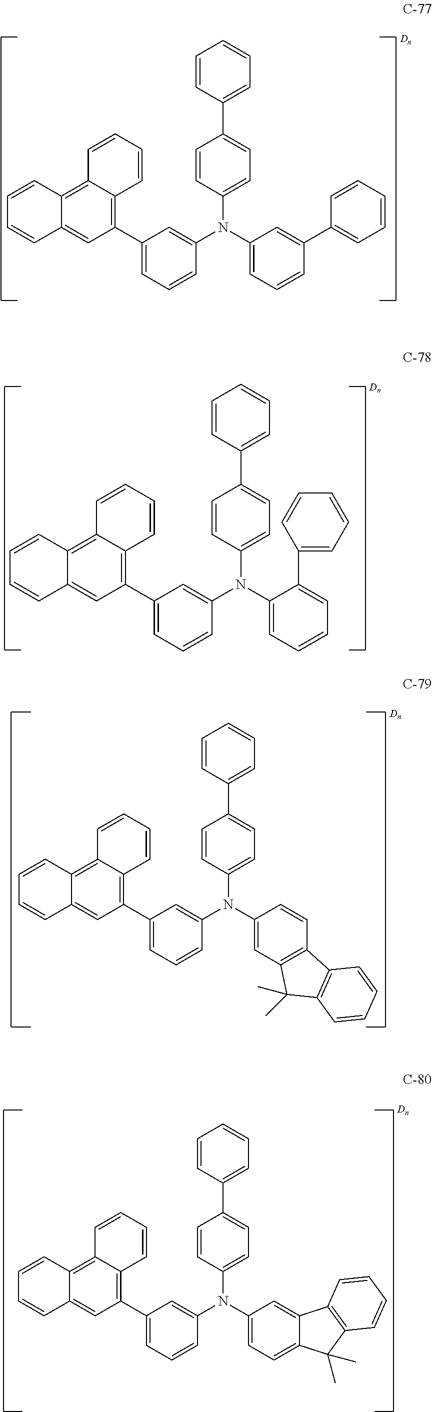

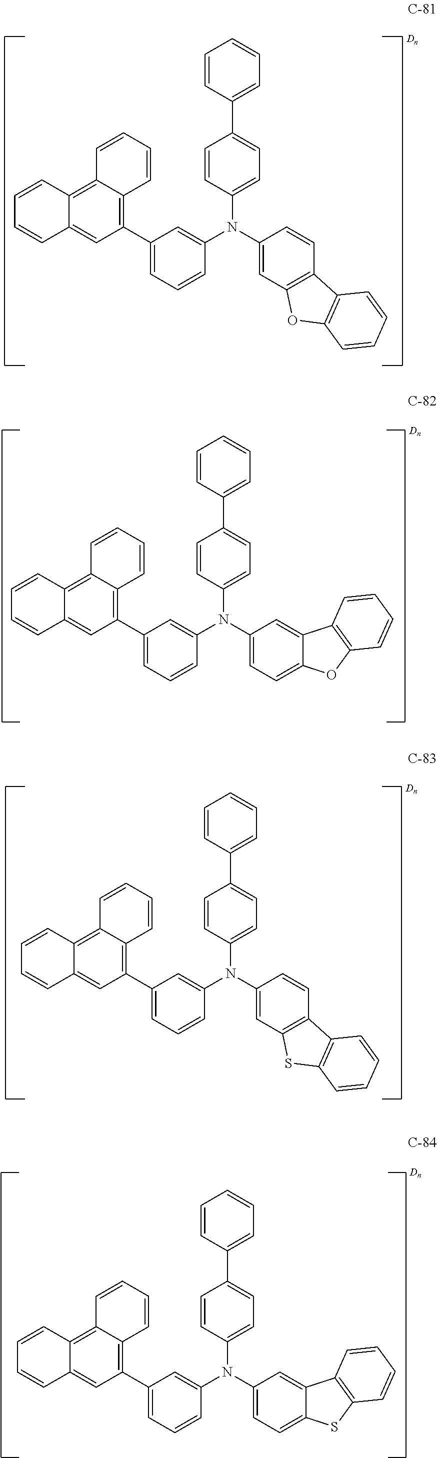

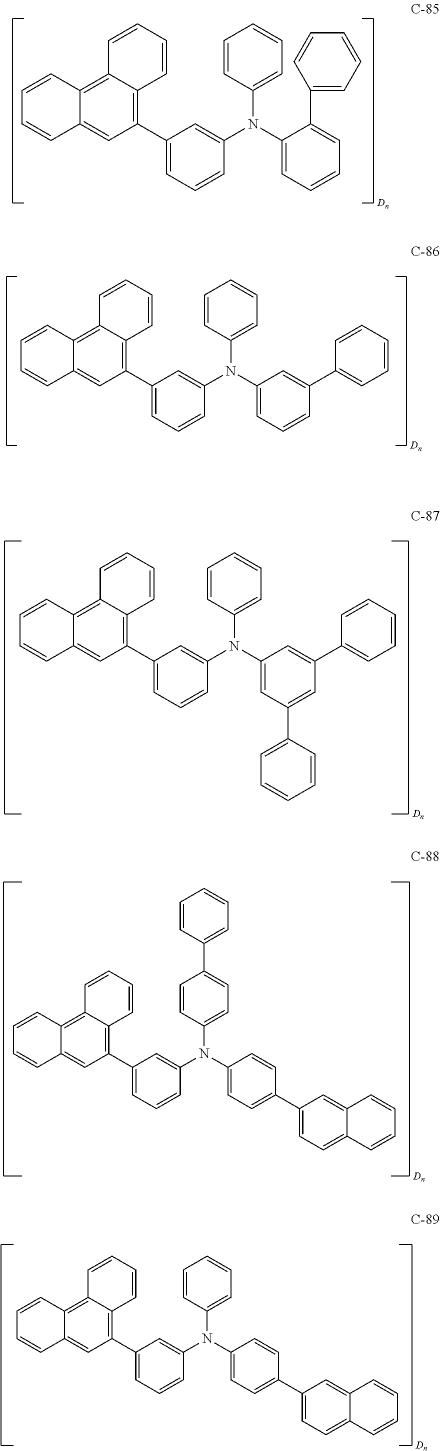

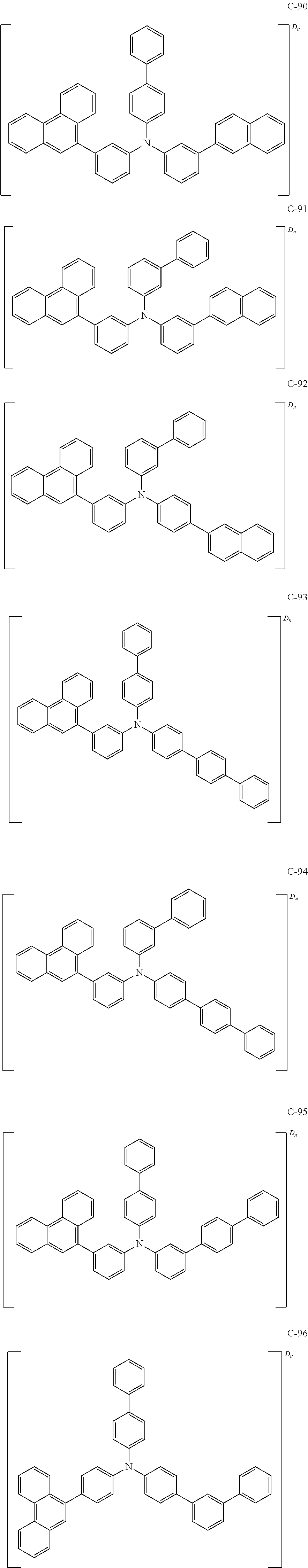

In the compounds above, Dₙ represents that n hydrogens are replaced with deuterium; and n represents an integer of 1 or more. The upper limit of n is determined by the number of hydrogens capable of being substituted in each compound. According to one embodiment of the present disclosure, n, each independently, represents preferably, an integer of 10 or more, and more preferably, an integer of 15 or more. When being deuterated to the number of the lower limit or more, the bond dissociation energy related to deuteration may increase to exhibit improved lifetime properties.

In formula 2, ring A, ring B, and ring C, each independently, represent a substituted or unsubstituted mono- or polycyclic (3- to 50-membered) alicyclic or aromatic ring, or the combination thereof, which may contain at least one heteroatom selected from B, N, O, S, Si, and P, and ring B and ring C may be linked to each other to form a fused ring. According to one embodiment of the present disclosure, ring A, ring B, and ring C, each independently, represent a substituted or unsubstituted mono- or polycyclic (5- to 40-membered) alicyclic or aromatic ring, or the combination thereof, which may contain at least one heteroatom selected from B, N, O, and S; and ring B and ring C may be linked to each other to form a fused ring. According to another embodiment of the present disclosure, ring A represents a substituted or unsubstituted mono- or polycyclic (6- to 30-membered) aromatic ring; ring B and ring C, each independently, represent a substituted or unsubstituted mono- or polycyclic (6- to 40-membered) aromatic ring, and the rings may contain at least one heteroatom selected from B, N, O, and S. Rings B and C may be linked to each other by a single bond, or by using —O— as a linker to form a fused ring. For example, ring A may be a substituted or unsubstituted benzene ring, an unsubstituted naphthalene ring, or a (21-membered)hetero ring substituted with a methyl(s). The substituent(s) of the substituted benzene ring may be at least one of the group consisting of deuterium; a methyl unsubstituted or substituted with a deuterium(s); a Cert-butyl; a diphenylamino unsubstituted or substituted with at least one of deuterium(s), a methyl(s), and a tert-butyl(s); a phenylnaphthylamino; a phenylbiphenylamino unsubstituted or substituted with a tert-butyl(s); a dinaphthylamino; a dibiphenylamino; a phenyldibenzofuranylamino; a substituted or unsubstituted phenyl; a naphthyl; a biphenyl; a terphenyl; a triphenylenyl; a carbazolyl; a phenoxazinyl; a phenothiazinyl; a 9,9-dimethyl-dihydroacridinyl; and a dimethylxanthenyl. The substituent(s) of the substituted phenyl may be at least one of the group consisting of a methyl, a carbazolyl, a dibenzofuranyl, a diphenylamino, a phenoxazinyl, a phenothiazinyl, and a 9,9-dimethyl-dihydroacridinyl. For example, ring B and ring C, each independently, may be a substituted or unsubstituted benzene ring, an unsubstituted naphthalene ring, an unsubstituted dibenzothiophene ring, an unsubstituted dibenzofuran ring, a carbazole ring substituted with at least one of a phenyl(s) and a diphenylamino(s), a (21-membered)hetero ring containing B and N and substituted with at least one of a methyl(s) and a phenyl(s), a (25-membered)hetero ring containing B and N and substituted with a phenyl(s), or a (36-membered)hetero ring containing B and N and substituted with a methyl(s); and the substituent(s) of the substituted benzene ring may be at least one of the group consisting of deuterium, a methyl, a tent-butyl, a phenyl, a naphthyl, a substituted or unsubstituted diphenylamino, a phenylnaphthylamino, a dibiphenylamino, a dinaphthylamino, a phenyldibenzofuranylamino, a carbazolylphenylamino substituted with a phenyl(s), and a dihydroacridinylphenylamino substituted with a methyl(s); and the substituent(s) of the substituted diphenylamino may be at least one of a methyl(s) and a diphenylamino(s).

In formula 2, Y₁ represents B; and X₁ and X₂, each independently, represent NR or O, in which R, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (3- to 30-membered)heteroaryl(C6-C30)arylamino; or may be linked to at least one of ring A, ring B, and ring C to form a fused ring(s). According to one embodiment of the present disclosure, R, each independently, represents hydrogen, deuterium, a substituted or unsubstituted (C1-C20)alkyl, a substituted or unsubstituted (C6-C25)aryl, or a substituted or unsubstituted (3- to 25-membered)heteroaryl; or may be linked to at least one of ring A, ring B and ring C to form a ring(s). According to another embodiment of the present disclosure, R, each independently, represents hydrogen; deuterium; an unsubstituted (C1-C10)alkyl, a (C6-C18)aryl unsubstituted or substituted with at least one of deuterium(s), a (C1-C10)alkyl(s), and a di(C6-C18)arylamino(s); or a (5- to 20-membered)heteroaryl substituted with a (C6-C18)aryl(s); or may be linked to at least one of ring A, ring B and ring C to form a ring(s). For example, R, each independently, may be hydrogen; deuterium; a phenyl unsubstituted or substituted with at least one of deuterium, a methyl(s), and a tert-butyl(s); a naphthyl; a biphenyl unsubstituted or substituted with a diphenylamino(s); a triphenylenyl; or a carbazolyl substituted with a phenyl(s); or may be linked to at least one of ring A, ring B and ring C via a single bond or via O, S, B, or isopropylene as a linker to form a fused ring(s).

According to one embodiment of the present disclosure, formula 2 may be represented by the following formula 2-1.

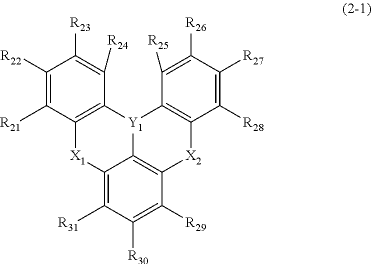

In formula 2-1, Y₁, X₁, and X₂, each independently, are as defined in formula 2.

In formula 2-1, R₂₁ to R₃₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (3- to 30-membered)heteroaryl(C6-C30)arylamino, or adjacent ones of R₂₁ to R₃₁ may be linked to each other to form a ring(s). According to one embodiment of the present disclosure, R₂₁ to R₃₁, each independently, represent hydrogen, deuterium, a substituted or unsubstituted (C1-C20)alkyl, a substituted or unsubstituted (C6-C25)aryl, a substituted or unsubstituted (5- to 20-membered)heteroaryl, a substituted or unsubstituted mono- or di-(C6-C25)arylamino, or a substituted or unsubstituted (5- to 20-membered)heteroaryl(C6-C25)arylamino; or adjacent ones of R₂₁ to R₃₁ may be linked to each other to form a ring(s). According to another embodiment of the present disclosure, R₂₁ to R₃₁, each independently, represent hydrogen; deuterium; a (C1-C10)alkyl unsubstituted or substituted with deuterium(s); a (C6-C18)aryl unsubstituted or substituted with at least one of deuterium(s), a (C1-C10)alkyl(s), a (13- to 18-membered)heteroaryl(s), and a di(C6-C18)arylamino(s); a (5- to 18-membered)heteroaryl unsubstituted or substituted with at least one of deuterium, and a (C1-C10)alkyl(s); a mono- or di-(C6-C18)arylamino unsubstituted or substituted with at least one of deuterium(s), a (C1-C10)alkyl(s), a di(C6-C18)arylamino(s), and a (13- to 20-membered)heteroaryl(s); or a (5- to 20-membered)heteroaryl(C6-C25)arylamino unsubstituted or substituted with at least one of a (C1-C10)alkyl(s) and a (C6-C18)aryl(s); or adjacent ones of R₂₁ to R₃₁ may be linked to each other to form a ring(s). For example, R₂₁ to R₃₁, each independently, may be hydrogen; deuterium; a methyl unsubstituted or substituted with a deuterium(s); a tert-butyl; a substituted or unsubstituted phenyl; a naphthyl; a biphenyl; a terphenyl; a triphenylenyl; a carbazolyl; a phenoxazinyl; a phenothiazinyl; a 9,9-dimethyl-dihydroacridinyl; a dimethylxanthenyl; a diphenylamino unsubstituted or substituted with at least one of deuterium(s), a methyl(s), a tert-butyl(s), and a diphenylamino(s); a phenylnaphthylamino; a phenylbiphenylamino unsubstituted or substituted with a tert-butyl(s); a dinaphthylamino; a dibiphenylamino; a carbazolylphenylamino substituted with a phenyl(s); a dibenzofuranylphenylamino; a dihydroacridinylphenylamino substituted with a methyl(s); or a (17- to 21-membered)heteroaryl substituted with at least one of a methyl(s) and a phenyl(s); or adjacent ones of R₂₁ to R₃₁ may be linked to each other to form a benzene ring, an indole ring substituted with at least one of a phenyl(s) and a diphenylamino(s), a benzofuran ring, a benzothiophene ring, a (17- to 32-membered)hetero ring containing B and N and substituted with at least one of a methyl(s) and a phenyl(s). In addition, R₂₄ and R₂₅ may be linked via —O— as a linker. The substituent(s) of the substituted phenyl may be at least one of a methyl(s), a carbazolyl(s), a dibenzofuranyl(s), a diphenylamino(s), a phenoxazinyl(s), a phenothiazinyl(s), and a 9,9-dimethyl-dihydroacridinyl(s).

The compound represented by formula 2 may be at least one selected from the group consisting of the following compounds, but is not limited thereto.

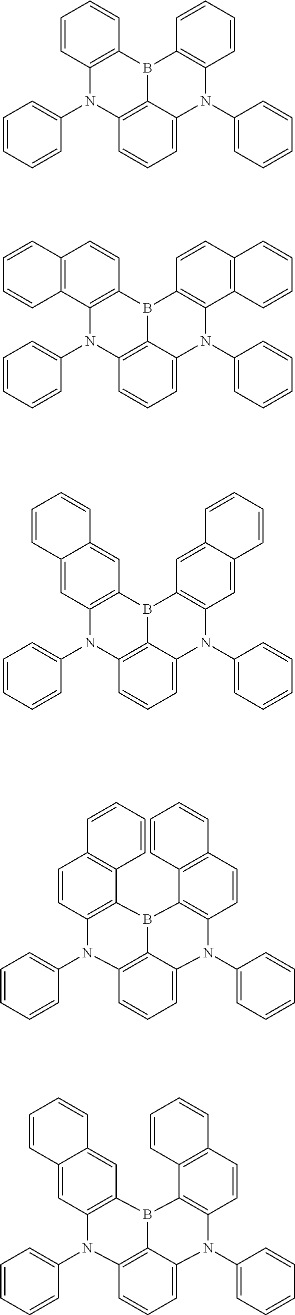 [SMILES: c1ccc(N2c3ccccc3B3c4ccccc4N(c4ccccc4)c4cccc2c43)cc1 ; c1ccc(N2c3cccc4c3B(c3ccc5ccccc5c32)c2ccc3ccccc3c2N4c2ccccc2)cc1 ; c1ccc(N2c3cc4ccccc4cc3B3c4cc5ccccc5cc4N(c4ccccc4)c4cccc2c43)cc1 ; c1ccc(N2c3cccc4c3B(c3c2ccc2ccccc32)c2c(ccc3ccccc23)N4c2ccccc2)cc1 ; c1ccc(N2c3cc4ccccc4cc3B3c4c2cccc4N(c2ccccc2)c2ccc4ccccc4c23)cc1]

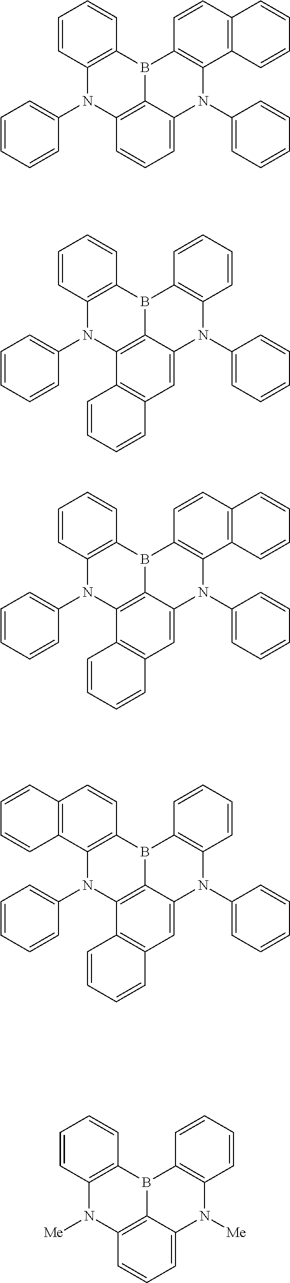 [SMILES: c1ccc(N2c3ccccc3B3c4ccc5ccccc5c4N(c4ccccc4)c4cccc2c43)cc1 ; c1ccc(N2c3ccccc3B3c4ccccc4N(c4ccccc4)c4c3c2cc2ccccc42)cc1 ; c1ccc(N2c3cc4ccccc4c4c3B(c3ccccc3N4c3ccccc3)c3ccc4ccccc4c32)cc1 ; c1ccc(N2c3ccccc3B3c4ccc5ccccc5c4N(c4ccccc4)c4c3c2cc2ccccc42)cc1 ; CN1c2ccccc2B2c3ccccc3N(C)c3cccc1c32]

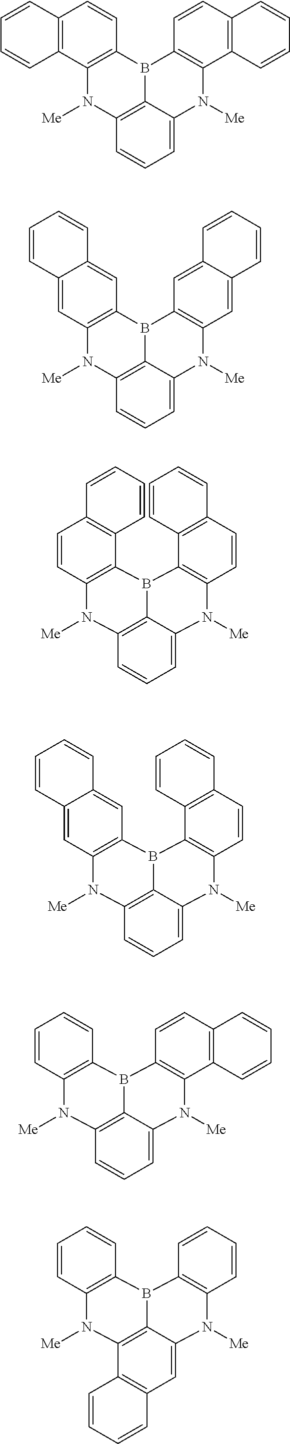 [SMILES: CN1c2cccc3c2B(c2ccc4ccccc4c21)c1ccc2ccccc2c1N3C ; CN1c2cc3ccccc3cc2B2c3cc4ccccc4cc3N(C)c3cccc1c32 ; CN1c2cccc3c2B(c2c1ccc1ccccc21)c1c(ccc2ccccc12)N3C ; CN1c2cc3ccccc3cc2B2c3c1cccc3N(C)c1ccc3ccccc3c12 ; CN1c2ccccc2B2c3ccc4ccccc4c3N(C)c3cccc1c32 ; CN1c2ccccc2B2c3ccccc3N(C)c3c2c1cc1ccccc31]

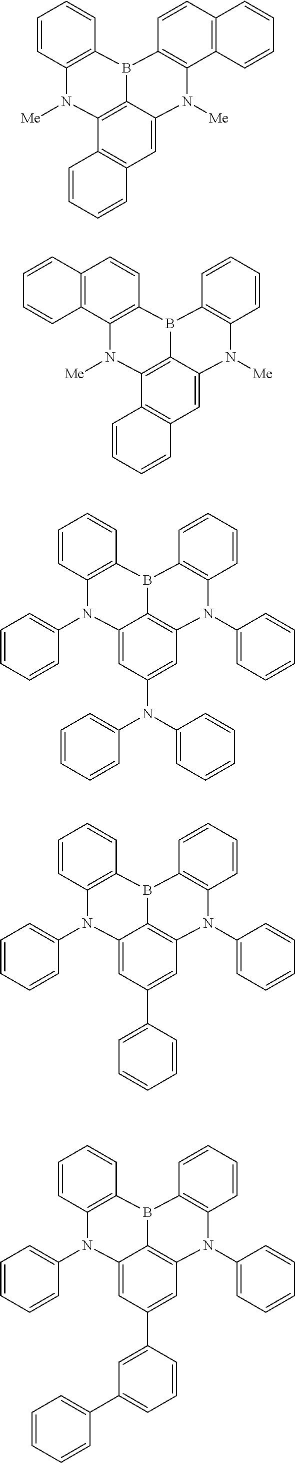

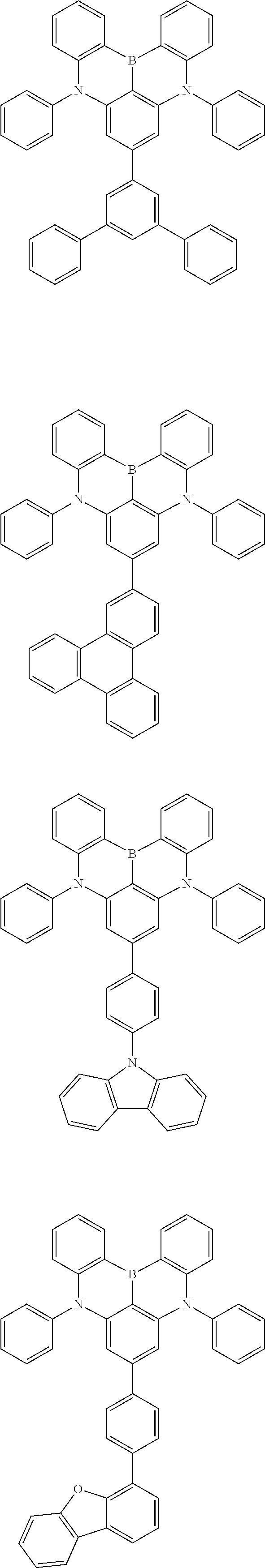 [SMILES: c1ccc(-c2cc(-c3ccccc3)cc(-c3cc4c5c(c3)N(c3ccccc3)c3ccccc3B5c3ccccc3N4c3ccccc3)c2)cc1 ; c1ccc(N2c3ccccc3B3c4ccccc4N(c4ccccc4)c4cc(-c5ccc6c7ccccc7c7ccccc7c6c5)cc2c43)cc1 ; c1ccc(N2c3ccccc3B3c4ccccc4N(c4ccccc4)c4cc(-c5ccc(-n6c7ccccc7c7ccccc76)cc5)cc2c43)cc1 ; c1ccc(N2c3ccccc3B3c4ccccc4N(c4ccccc4)c4cc(-c5ccc(-c6cccc7c6oc6ccccc67)cc5)cc2c43)cc1]

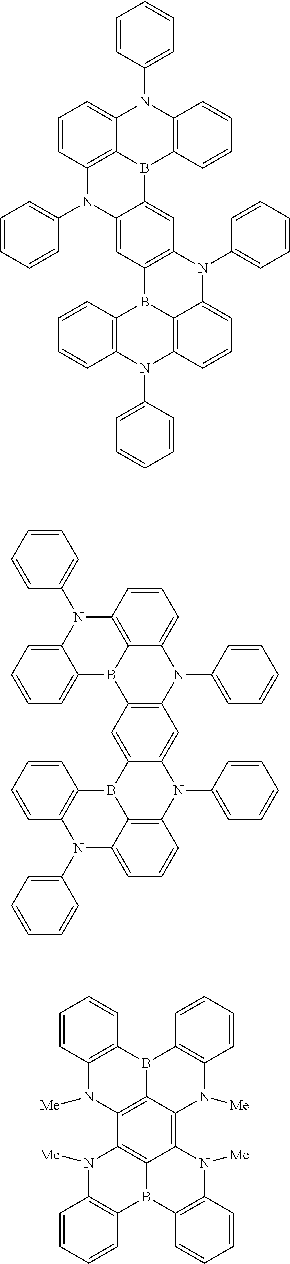 [SMILES: c1ccc(N2c3ccccc3B3c4cc5c(cc4N(c4ccccc4)c4cccc2c43)B2c3ccccc3N(c3ccccc3)c3cccc(c32)N5c2ccccc2)cc1 ; c1ccc(N2c3ccccc3B3c4cc5c(cc4N(c4ccccc4)c4cccc2c43)N(c2ccccc2)c2cccc3c2B5c2ccccc2N3c2ccccc2)cc1 ; CN1c2ccccc2B2c3ccccc3N(C)c3c2c1c1c2c3N(C)c3ccccc3B2c2ccccc2N1C]

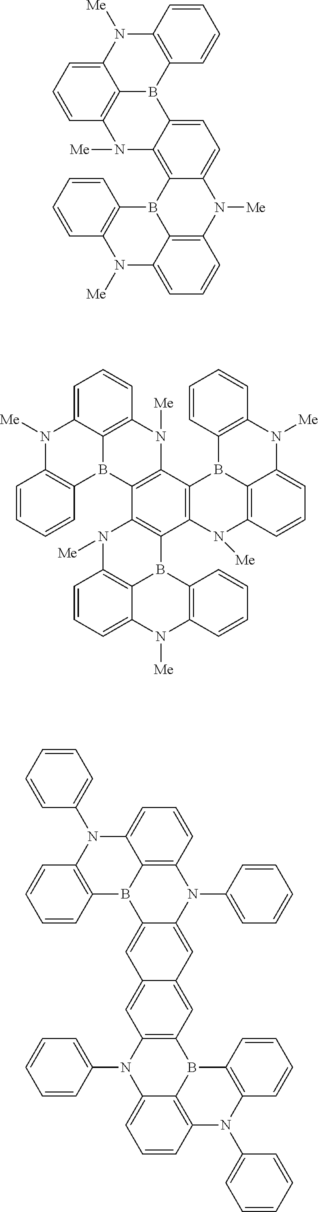 [SMILES: CN1c2ccccc2B2c3c1cccc3N(C)c1ccc3c(c12)N(C)c1cccc2c1B3c1ccccc1N2C ; CN1c2ccccc2B2c3c1cccc3N(C)c1c3c(c4c(c12)N(C)c1cccc2c1B4c1ccccc1N2C)N(C)c1cccc2c1B3c1ccccc1N2C ; c1ccc(N2c3ccccc3B3c4cc5cc6c(cc5cc4N(c4ccccc4)c4cccc2c43)B2c3ccccc3N(c3ccccc3)c3cccc(c32)N6c2ccccc2)cc1]

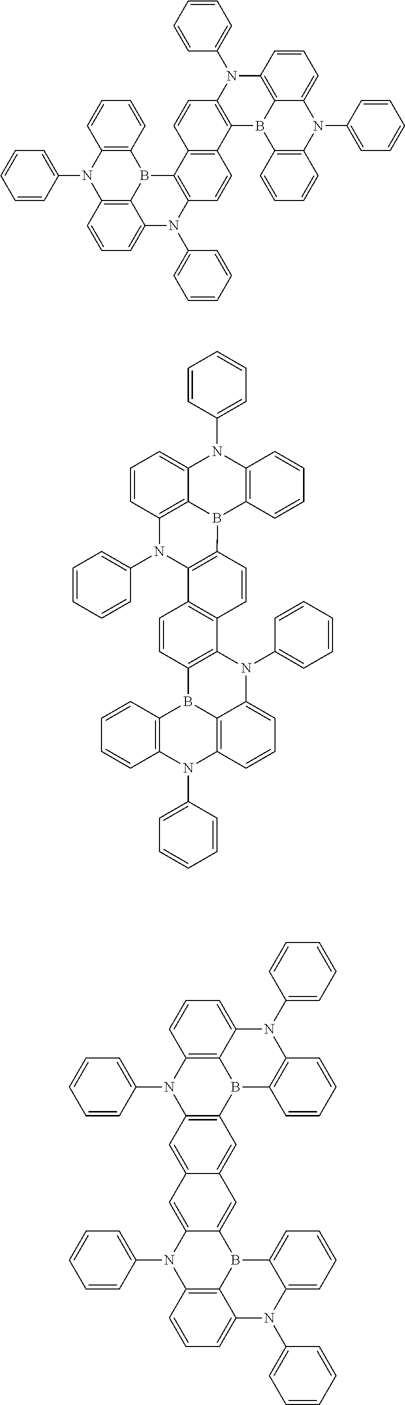 [SMILES: c1ccc(N2c3ccccc3B3c4c2cccc4N(c2ccccc2)c2ccc4c5c(ccc4c23)N(c2ccccc2)c2cccc3c2B5c2ccccc2N3c2ccccc2)cc1 ; c1ccc(N2c3ccccc3B3c4ccc5c6c(ccc5c4N(c4ccccc4)c4cccc2c43)B2c3ccccc3N(c3ccccc3)c3cccc(c32)N6c2ccccc2)cc1 ; c1ccc(N2c3ccccc3B3c4cc5cc6c(cc5cc4N(c4ccccc4)c4cccc2c43)N(c2ccccc2)c2cccc3c2B6c2ccccc2N3c2ccccc2)cc1]

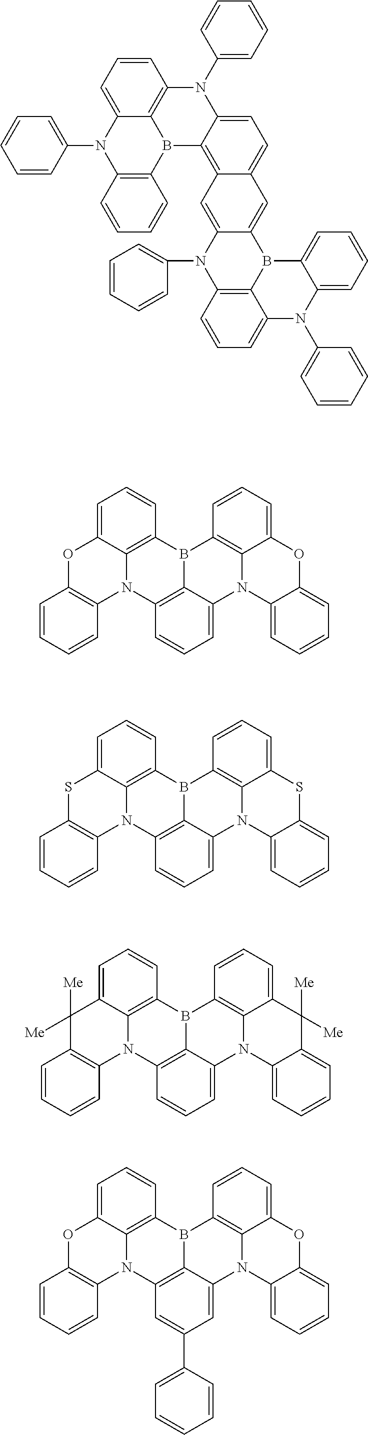 [SMILES: c1ccc(N2c3ccccc3B3c4cc5ccc6c(c5cc4N(c4ccccc4)c4cccc2c43)B2c3ccccc3N(c3ccccc3)c3cccc(c32)N6c2ccccc2)cc1 ; c1ccc2c(c1)Oc1cccc3c1N2c1cccc2c1B3c1cccc3c1N2c1ccccc1O3 ; c1ccc2c(c1)Sc1cccc3c1N2c1cccc2c1B3c1cccc3c1N2c1ccccc1S3 ; CC1(C)c2ccccc2N2c3cccc4c3B(c3cccc1c32)c1cccc2c1N4c1ccccc1C2(C)C ; c1ccc(-c2cc3c4c(c2)N2c5ccccc5Oc5cccc(c52)B4c2cccc4c2N3c2ccccc2O4)cc1]

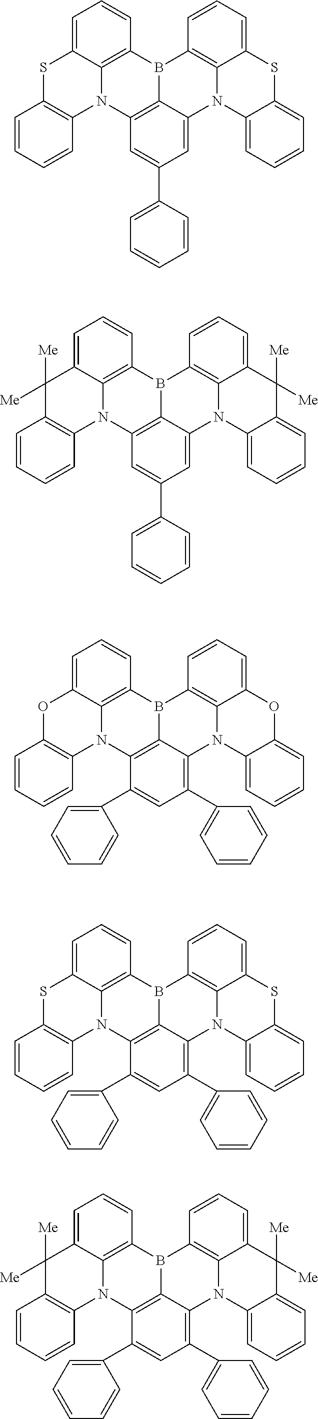

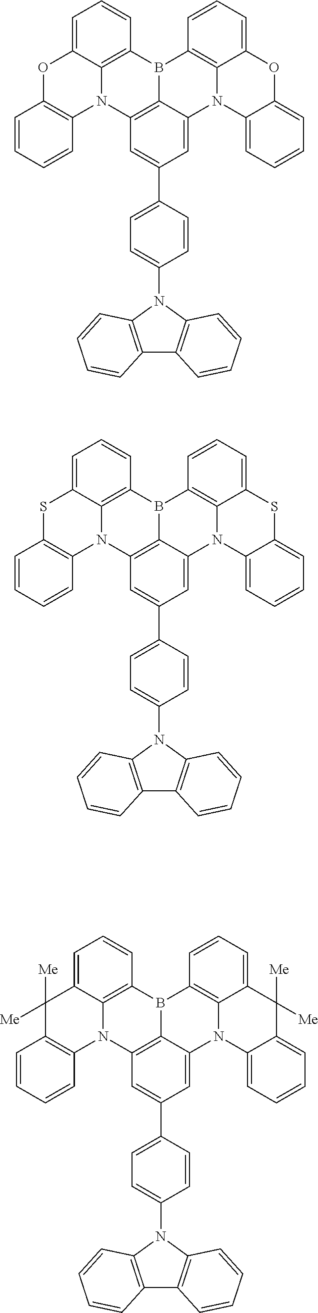 [SMILES: c1ccc2c(c1)Oc1cccc3c1N2c1cc(-c2ccc(-n4c5ccccc5c5ccccc54)cc2)cc2c1B3c1cccc3c1N2c1ccccc1O3 ; c1ccc2c(c1)Sc1cccc3c1N2c1cc(-c2ccc(-n4c5ccccc5c5ccccc54)cc2)cc2c1B3c1cccc3c1N2c1ccccc1S3 ; CC1(C)c2ccccc2N2c3cc(-c4ccc(-n5c6ccccc6c6ccccc65)cc4)cc4c3B(c3cccc1c32)c1cccc2c1N4c1ccccc1C2(C)C]

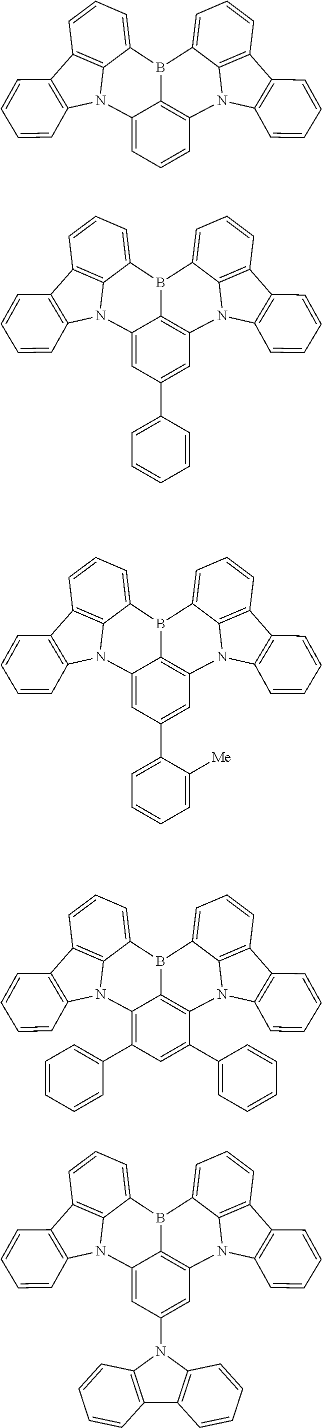

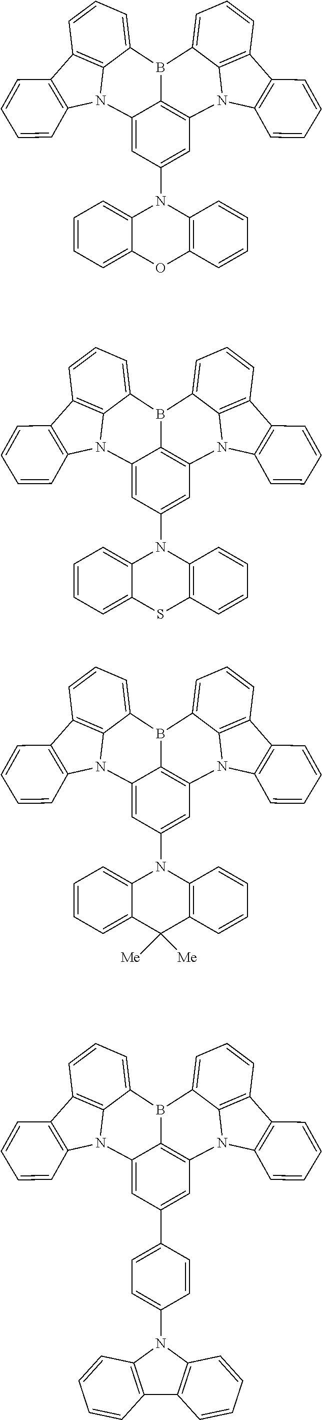 [SMILES: c1ccc2c(c1)Oc1ccccc1N2c1cc2c3c(c1)-n1c4ccccc4c4cccc(c41)B3c1cccc3c4ccccc4n-2c13 ; c1ccc2c(c1)Sc1ccccc1N2c1cc2c3c(c1)-n1c4ccccc4c4cccc(c41)B3c1cccc3c4ccccc4n-2c13 ; CC1(C)c2ccccc2N(c2cc3c4c(c2)-n2c5ccccc5c5cccc(c52)B4c2cccc4c5ccccc5n-3c24)c2ccccc21 ; c1ccc2c(c1)c1ccccc1n2-c1ccc(-c2cc3c4c(c2)-n2c5ccccc5c5cccc(c52)B4c2cccc4c5ccccc5n-3c24)cc1]

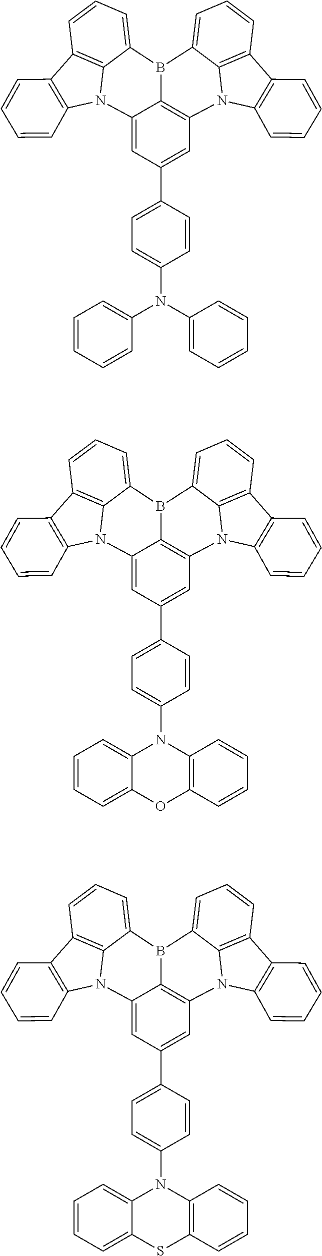 [SMILES: c1ccc(N(c2ccccc2)c2ccc(-c3cc4c5c(c3)-n3c6ccccc6c6cccc(c63)B5c3cccc5c6ccccc6n-4c35)cc2)cc1 ; c1ccc2c(c1)Oc1ccccc1N2c1ccc(-c2cc3c4c(c2)-n2c5ccccc5c5cccc(c52)B4c2cccc4c5ccccc5n-3c24)cc1 ; c1ccc2c(c1)Sc1ccccc1N2c1ccc(-c2cc3c4c(c2)-n2c5ccccc5c5cccc(c52)B4c2cccc4c5ccccc5n-3c24)cc1]

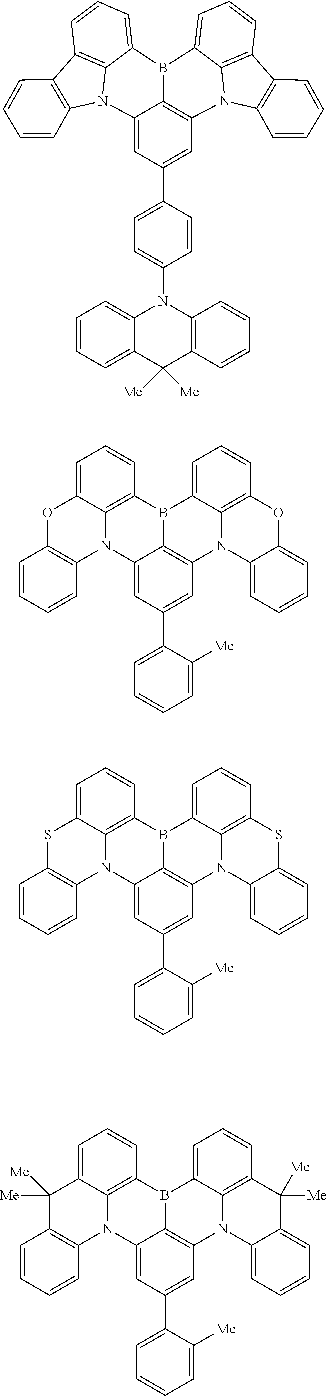 [SMILES: CC1(C)c2ccccc2N(c2ccc(-c3cc4c5c(c3)-n3c6ccccc6c6cccc(c63)B5c3cccc5c6ccccc6n-4c35)cc2)c2ccccc21 ; Cc1ccccc1-c1cc2c3c(c1)N1c4ccccc4Oc4cccc(c41)B3c1cccc3c1N2c1ccccc1O3 ; Cc1ccccc1-c1cc2c3c(c1)N1c4ccccc4Sc4cccc(c41)B3c1cccc3c1N2c1ccccc1S3 ; Cc1ccccc1-c1cc2c3c(c1)N1c4ccccc4C(C)(C)c4cccc(c41)B3c1cccc3c1N2c1ccccc1C3(C)C]

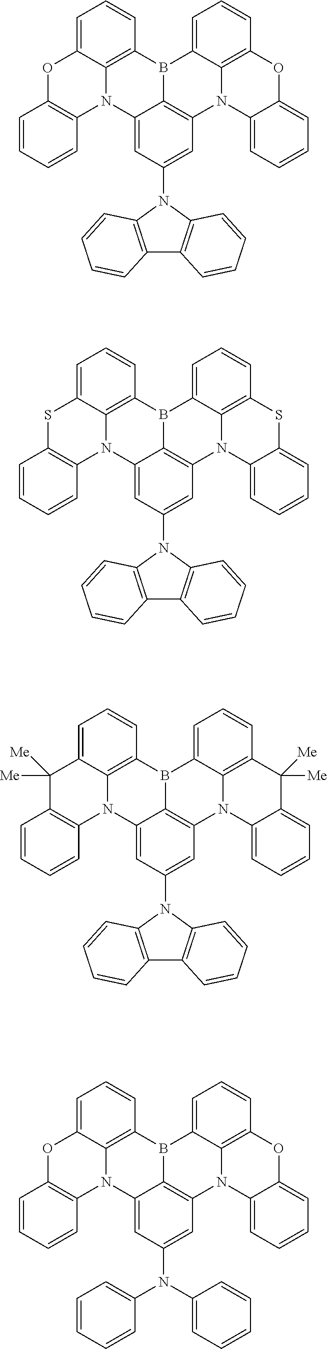 [SMILES: c1ccc2c(c1)Oc1cccc3c1N2c1cc(-n2c4ccccc4c4ccccc42)cc2c1B3c1cccc3c1N2c1ccccc1O3 ; c1ccc2c(c1)Sc1cccc3c1N2c1cc(-n2c4ccccc4c4ccccc42)cc2c1B3c1cccc3c1N2c1ccccc1S3 ; CC1(C)c2ccccc2N2c3cc(-n4c5ccccc5c5ccccc54)cc4c3B(c3cccc1c32)c1cccc2c1N4c1ccccc1C2(C)C ; c1ccc(N(c2ccccc2)c2cc3c4c(c2)N2c5ccccc5Oc5cccc(c52)B4c2cccc4c2N3c2ccccc2O4)cc1]

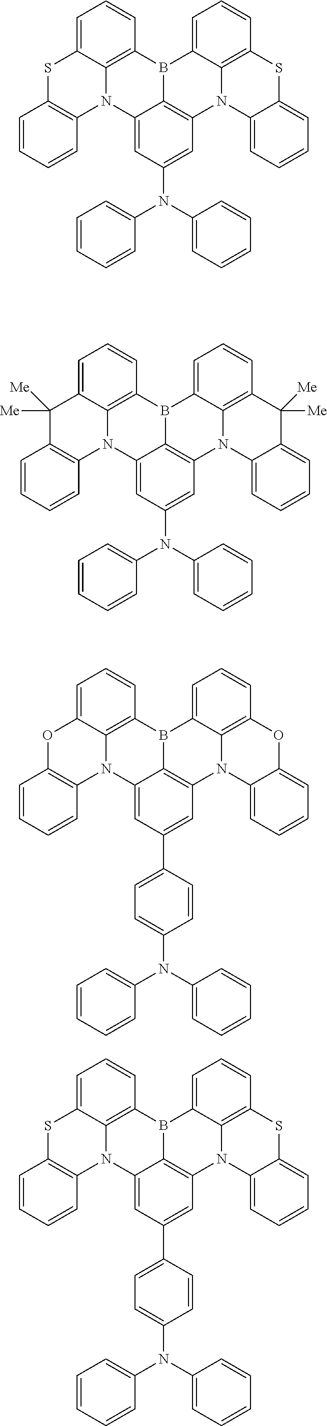

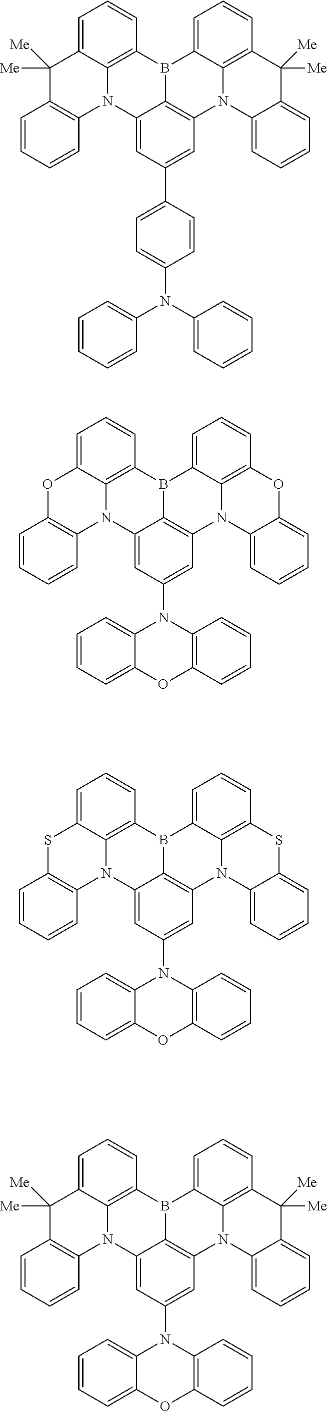 [SMILES: CC1(C)c2ccccc2N2c3cc(-c4ccc(N(c5ccccc5)c5ccccc5)cc4)cc4c3B(c3cccc1c32)c1cccc2c1N4c1ccccc1C2(C)C ; c1ccc2c(c1)Oc1ccccc1N2c1cc2c3c(c1)N1c4ccccc4Oc4cccc(c41)B3c1cccc3c1N2c1ccccc1O3 ; c1ccc2c(c1)Oc1ccccc1N2c1cc2c3c(c1)N1c4ccccc4Sc4cccc(c41)B3c1cccc3c1N2c1ccccc1S3 ; CC1(C)c2ccccc2N2c3cc(N4c5ccccc5Oc5ccccc54)cc4c3B(c3cccc1c32)c1cccc2c1N4c1ccccc1C2(C)C]

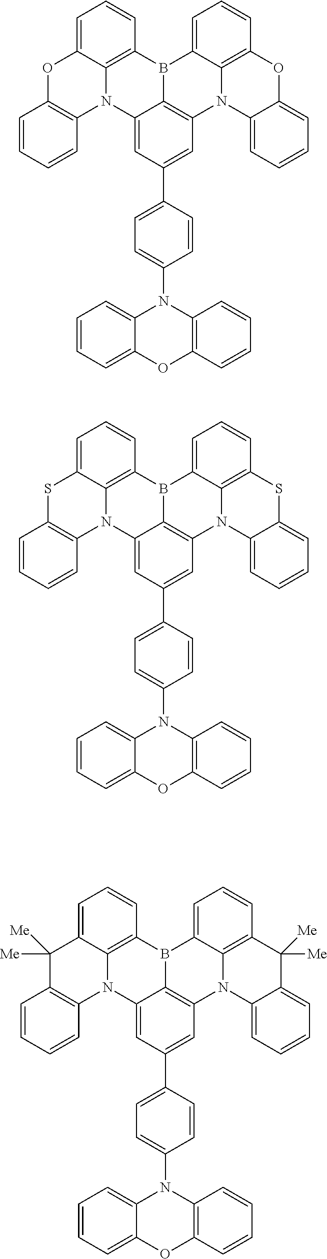 [SMILES: c1ccc2c(c1)Oc1ccccc1N2c1ccc(-c2cc3c4c(c2)N2c5ccccc5Oc5cccc(c52)B4c2cccc4c2N3c2ccccc2O4)cc1 ; c1ccc2c(c1)Oc1ccccc1N2c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccc(N5c6ccccc6Oc6ccccc65)cc4)cc4c3B(c3cccc1c32)c1cccc2c1N4c1ccccc1C2(C)C]

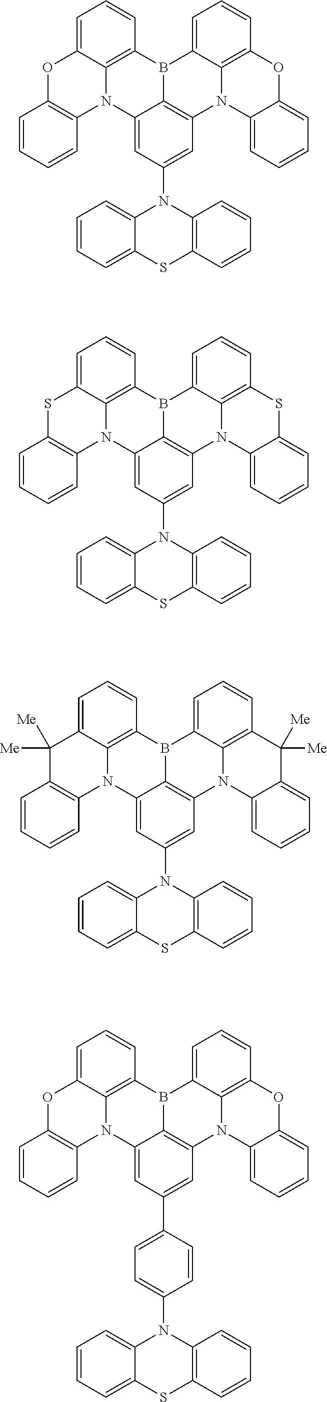 [SMILES: c1ccc2c(c1)Oc1cccc3c1N2c1cc(N2c4ccccc4Sc4ccccc42)cc2c1B3c1cccc3c1N2c1ccccc1O3 ; c1ccc2c(c1)Sc1ccccc1N2c1cc2c3c(c1)N1c4ccccc4Sc4cccc(c41)B3c1cccc3c1N2c1ccccc1S3 ; CC1(C)c2ccccc2N2c3cc(N4c5ccccc5Sc5ccccc54)cc4c3B(c3cccc1c32)c1cccc2c1N4c1ccccc1C2(C)C ; c1ccc2c(c1)Oc1cccc3c1N2c1cc(-c2ccc(N4c5ccccc5Sc5ccccc54)cc2)cc2c1B3c1cccc3c1N2c1ccccc1O3]

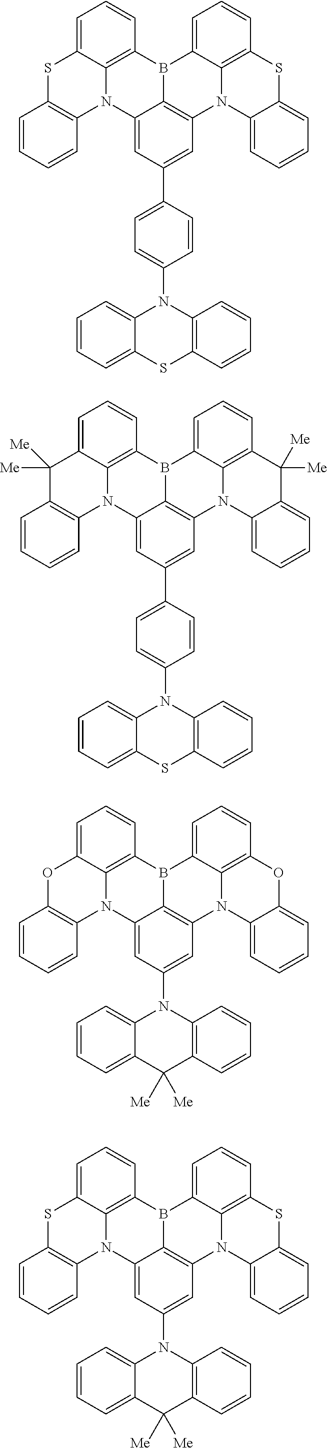

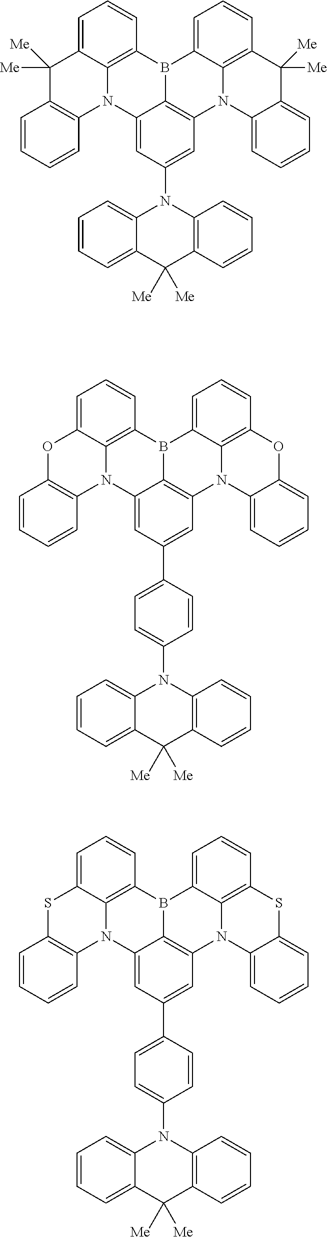 [SMILES: CC1(C)c2ccccc2N(c2cc3c4c(c2)N2c5ccccc5C(C)(C)c5cccc(c52)B4c2cccc4c2N3c2ccccc2C4(C)C)c2ccccc21 ; CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; CC1(C)c2ccccc2N(c2ccc(-c3cc4c5c(c3)N3c6ccccc6Sc6cccc(c63)B5c3cccc5c3N4c3ccccc3S5)cc2)c2ccccc21]

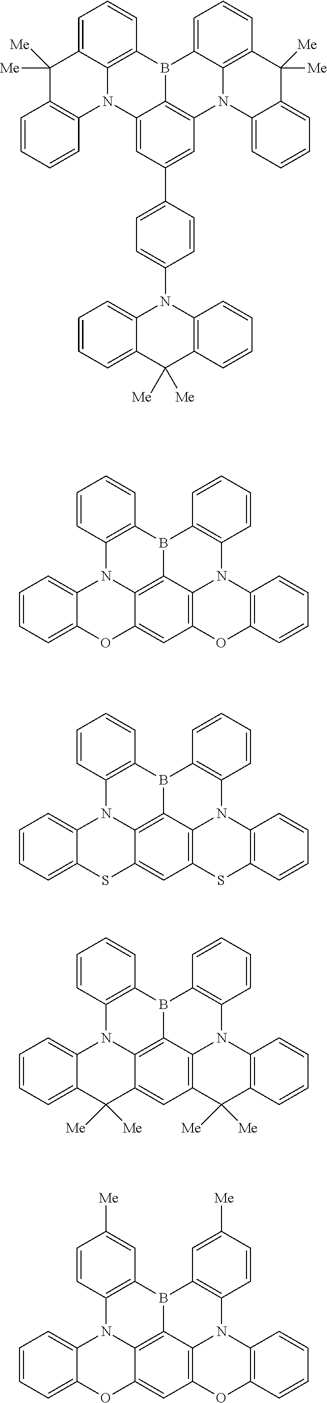 [SMILES: CC1(C)c2ccccc2N(c2ccc(-c3cc4c5c(c3)N3c6ccccc6C(C)(C)c6cccc(c63)B5c3cccc5c3N4c3ccccc3C5(C)C)cc2)c2ccccc21 ; c1ccc2c(c1)B1c3ccccc3-n3c4ccccc4oc4cc5oc6ccccc6n-2c5c1c43 ; c1ccc2c(c1)B1c3ccccc3-n3c4ccccc4sc4cc5sc6ccccc6n-2c5c1c43 ; CC1(C)c2ccccc2N2c3ccccc3B3c4ccccc4N4c5ccccc5C(C)(C)c5cc1c2c3c54 ; Cc1ccc2c(c1)B1c3cc(C)ccc3-n3c4ccccc4oc4cc5oc6ccccc6n-2c5c1c43]

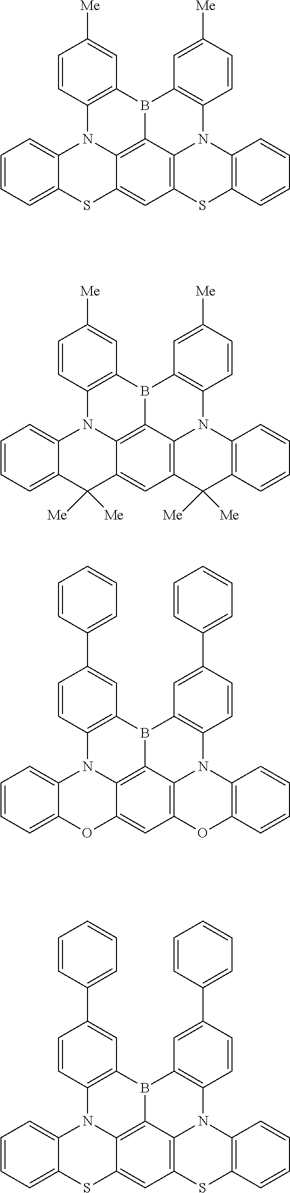 [SMILES: Cc1ccc2c(c1)B1c3cc(C)ccc3-n3c4ccccc4sc4cc5sc6ccccc6n-2c5c1c43 ; Cc1ccc2c(c1)B1c3cc(C)ccc3N3c4ccccc4C(C)(C)c4cc5c(c1c43)N2c1ccccc1C5(C)C ; c1ccc(-c2ccc3c(c2)B2c4cc(-c5ccccc5)ccc4-n4c5ccccc5oc5cc6oc7ccccc7n-3c6c2c54)cc1 ; c1ccc(-c2ccc3c(c2)B2c4cc(-c5ccccc5)ccc4-n4c5ccccc5sc5cc6sc7ccccc7n-3c6c2c54)cc1]

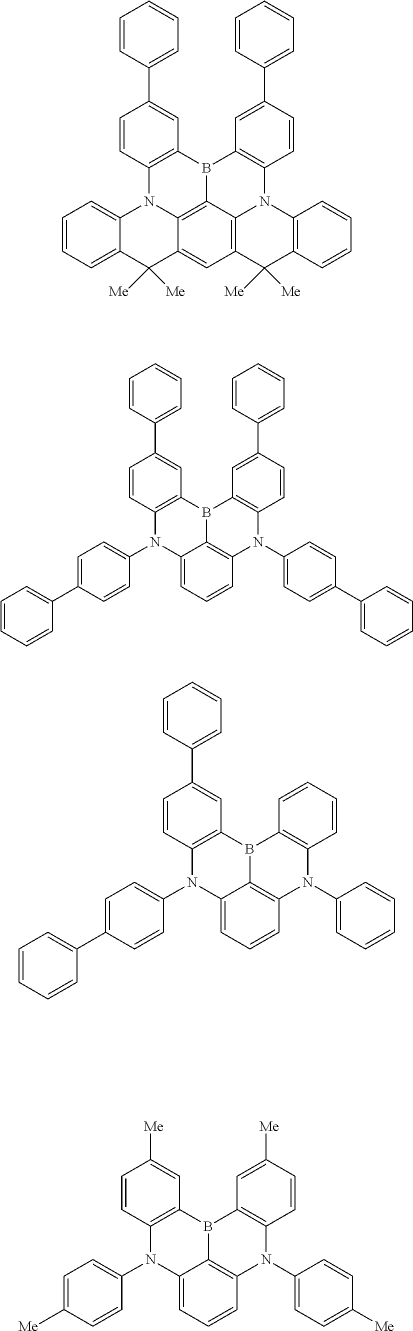 [SMILES: CC1(C)c2ccccc2N2c3ccc(-c4ccccc4)cc3B3c4cc(-c5ccccc5)ccc4N4c5ccccc5C(C)(C)c5cc1c2c3c54 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1 ; Cc1ccc(N2c3ccc(C)cc3B3c4cc(C)ccc4N(c4ccc(C)cc4)c4cccc2c43)cc1]

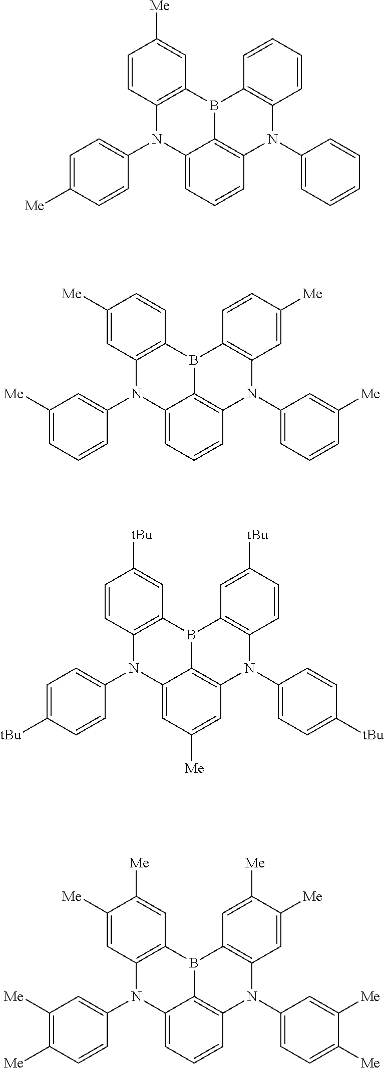 [SMILES: Cc1ccc(N2c3ccc(C)cc3B3c4ccccc4N(c4ccccc4)c4cccc2c43)cc1 ; Cc1cccc(N2c3cc(C)ccc3B3c4ccc(C)cc4N(c4cccc(C)c4)c4cccc2c43)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; Cc1ccc(N2c3cc(C)c(C)cc3B3c4cc(C)c(C)cc4N(c4ccc(C)c(C)c4)c4cccc2c43)cc1C]

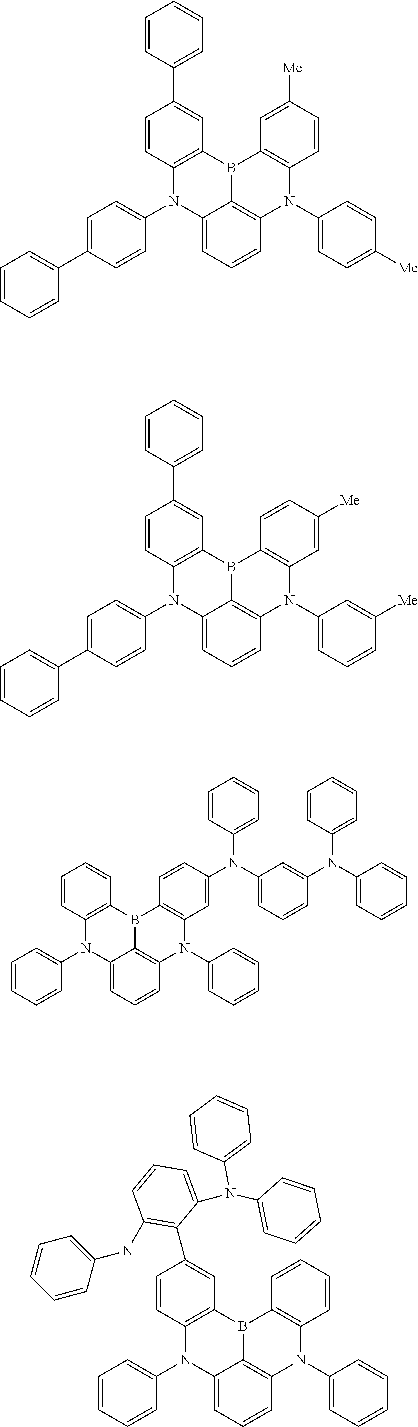 [SMILES: Cc1ccc(N2c3ccc(C)cc3B3c4cc(-c5ccccc5)ccc4N(c4ccc(-c5ccccc5)cc4)c4cccc2c43)cc1 ; Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; c1ccc(Nc2cccc(N(c3ccccc3)c3ccccc3)c2-c2ccc3c(c2)B2c4ccccc4N(c4ccccc4)c4cccc(c42)N3c2ccccc2)cc1]

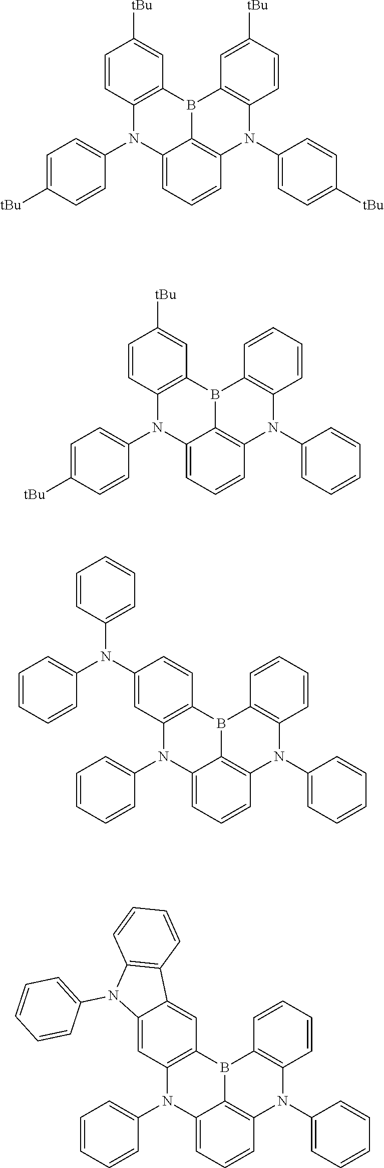 [SMILES: CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1 ; c1ccc(N2c3ccccc3B3c4cc5c6ccccc6n(-c6ccccc6)c5cc4N(c4ccccc4)c4cccc2c43)cc1]

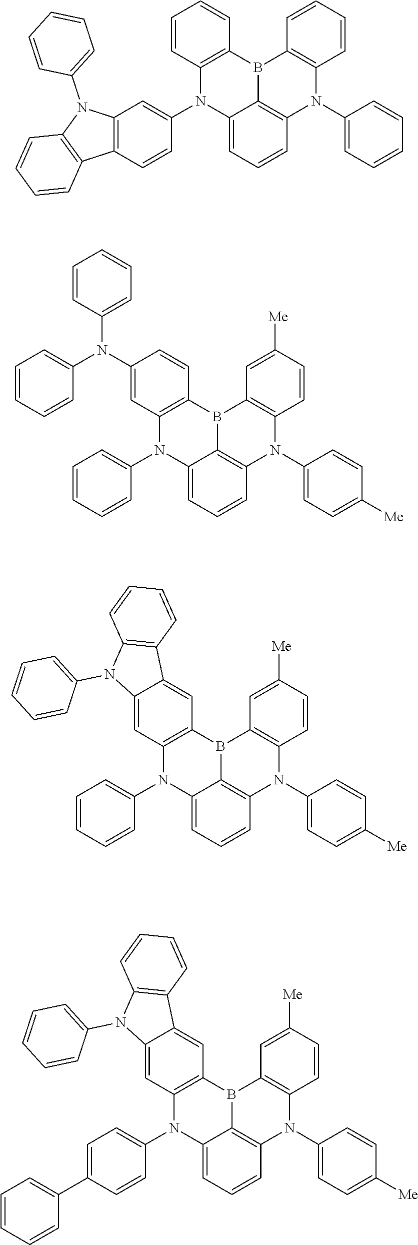 [SMILES: c1ccc(N2c3ccccc3B3c4ccccc4N(c4ccc5c6ccccc6n(-c6ccccc6)c5c4)c4cccc2c43)cc1 ; Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1 ; Cc1ccc(N2c3ccc(C)cc3B3c4cc5c6ccccc6n(-c6ccccc6)c5cc4N(c4ccc(-c5ccccc5)cc4)c4cccc2c43)cc1]

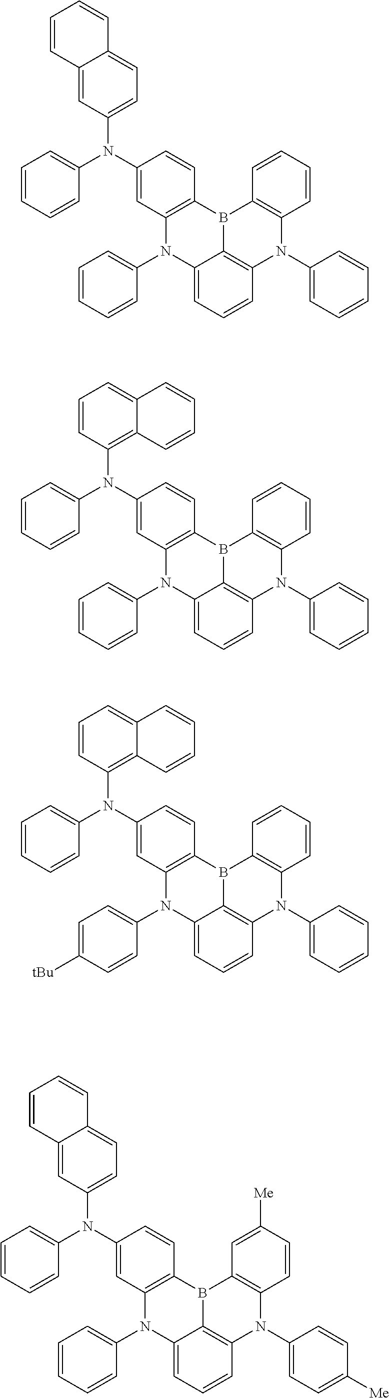 [SMILES: c1ccc(N(c2ccc3c(c2)N(c2ccccc2)c2cccc4c2B3c2ccccc2N4c2ccccc2)c2ccc3ccccc3c2)cc1 ; c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1 ; Cc1ccc(N2c3ccc(C)cc3B3c4ccc(N(c5ccccc5)c5ccc6ccccc6c5)cc4N(c4ccccc4)c4cccc2c43)cc1]

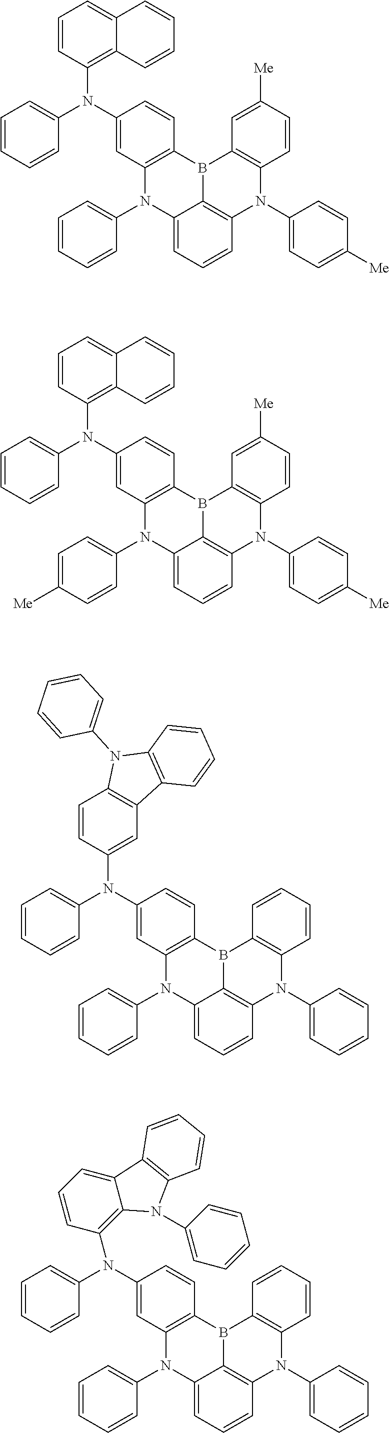 [SMILES: Cc1ccc(N2c3ccc(C)cc3B3c4ccc(N(c5ccccc5)c5cccc6ccccc56)cc4N(c4ccccc4)c4cccc2c43)cc1 ; Cc1ccc(N2c3ccc(C)cc3B3c4ccc(N(c5ccccc5)c5cccc6ccccc56)cc4N(c4ccc(C)cc4)c4cccc2c43)cc1 ; c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1]

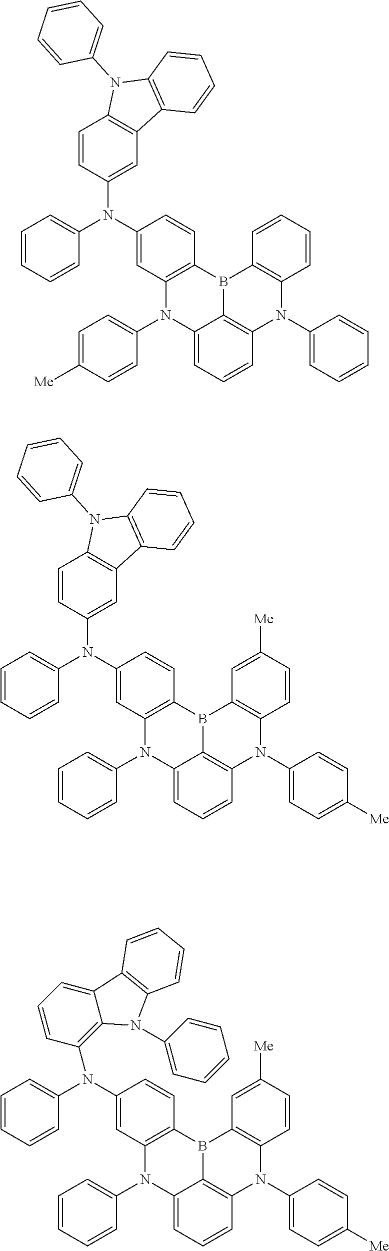 [SMILES: Cc1ccc(N2c3cc(N(c4ccccc4)c4ccc5c(c4)c4ccccc4n5-c4ccccc4)ccc3B3c4ccccc4N(c4ccccc4)c4cccc2c43)cc1 ; Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1]

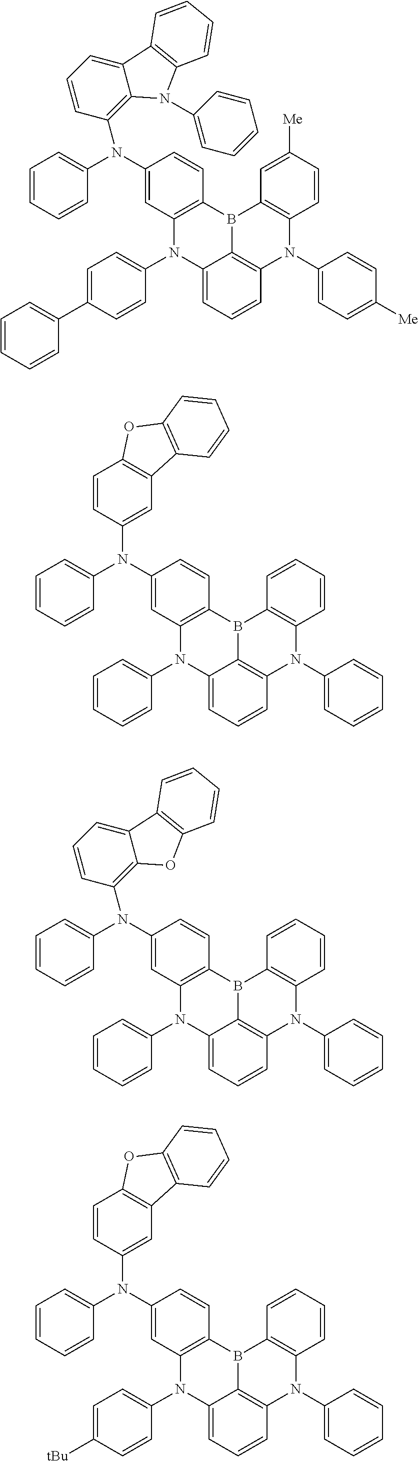 [SMILES: Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1]

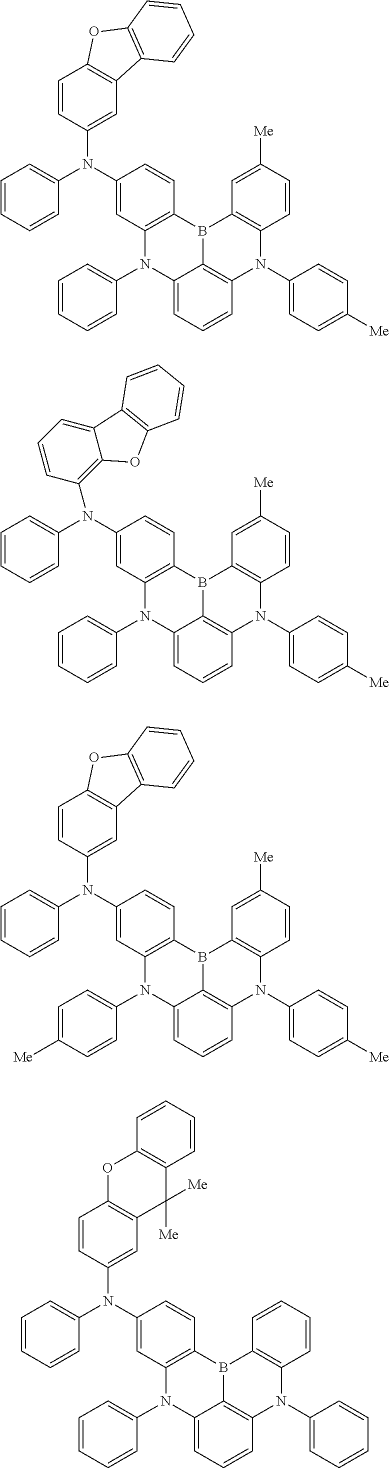 [SMILES: Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1 ; CC1(C)c2ccccc2Oc2ccc(N(c3ccccc3)c3ccc4c(c3)N(c3ccccc3)c3cccc5c3B4c3ccccc3N5c3ccccc3)cc21]

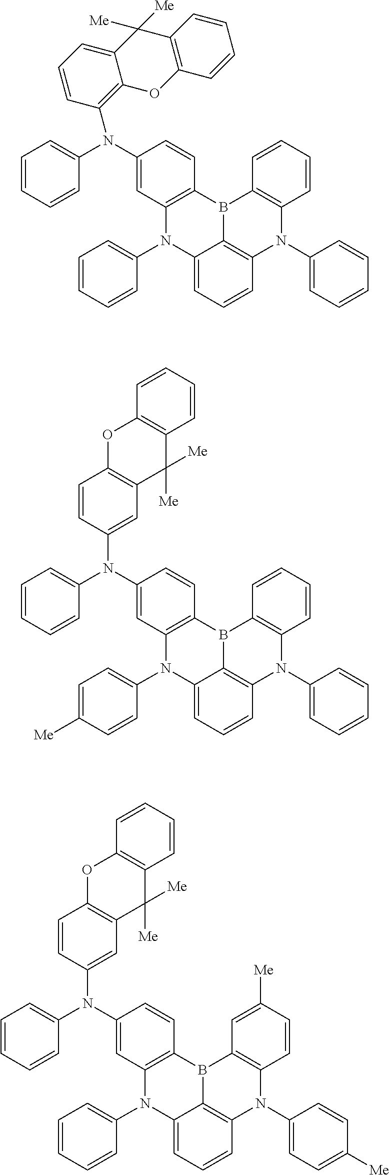 [SMILES: CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1]

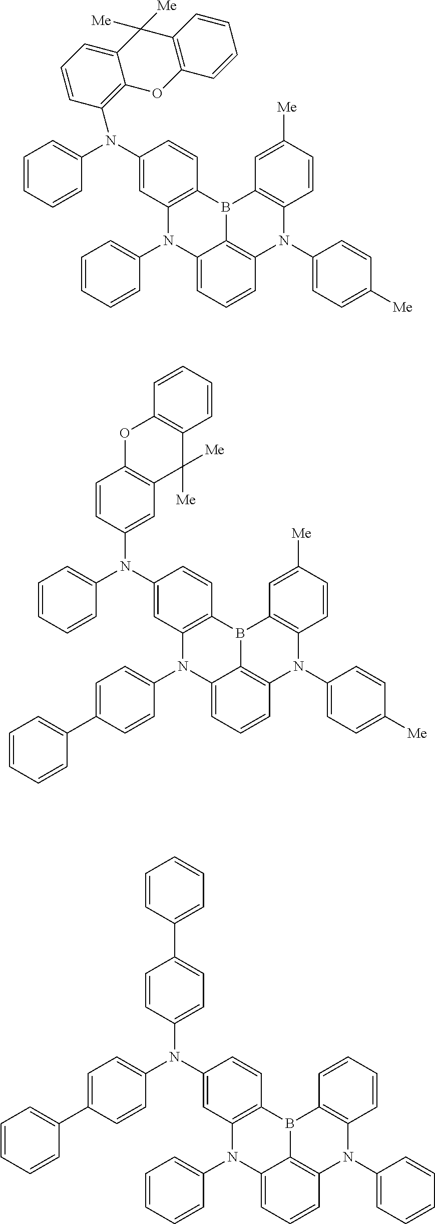 [SMILES: Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1 ; c1ccc(-c2ccc(N(c3ccc(-c4ccccc4)cc3)c3ccc4c(c3)N(c3ccccc3)c3cccc5c3B4c3ccccc3N5c3ccccc3)cc2)cc1]

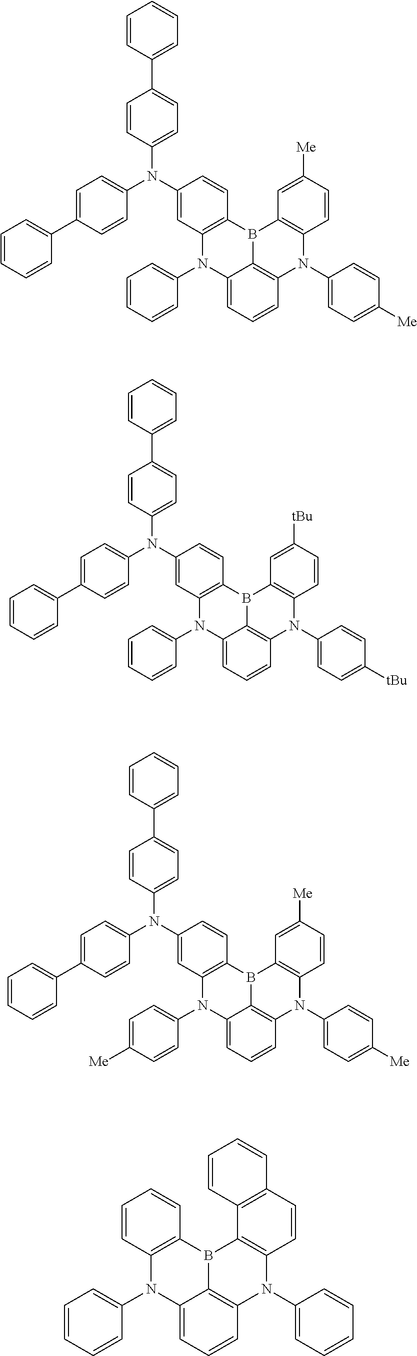 [SMILES: Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1 ; CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1 ; Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1 ; c1ccc(N2c3ccccc3B3c4c2cccc4N(c2ccccc2)c2ccc4ccccc4c23)cc1]

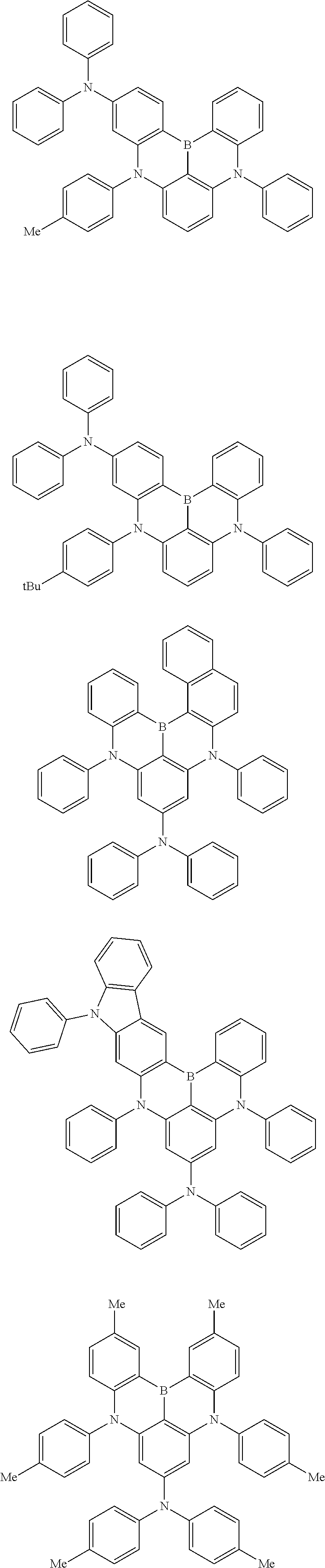

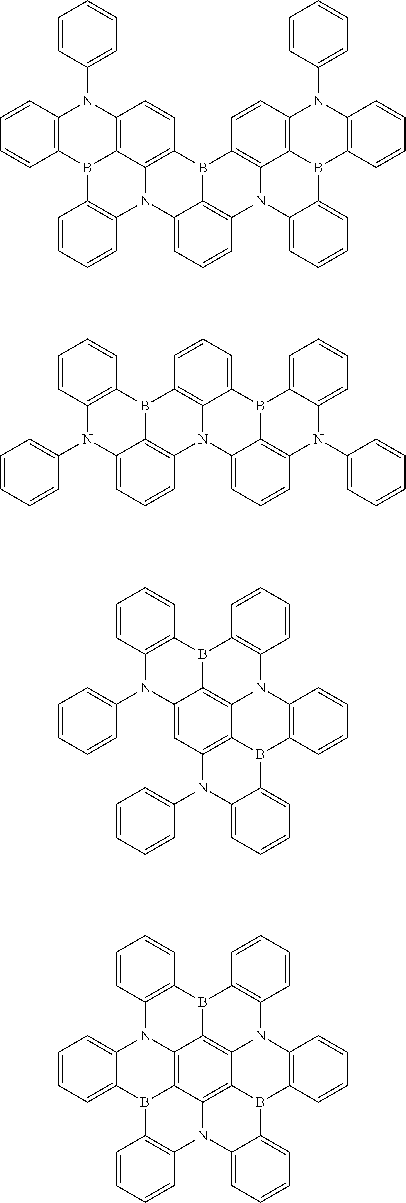 [SMILES: c1ccc(N2c3ccccc3B3c4ccccc4N4c5cccc6c5B(c5ccc2c3c54)c2ccc3c4c2N6c2ccccc2B4c2ccccc2N3c2ccccc2)cc1 ; c1ccc(N2c3ccccc3B3c4cccc5c4N(c4cccc2c43)c2cccc3c2B5c2ccccc2N3c2ccccc2)cc1 ; c1ccc(N2c3ccccc3B3c4ccccc4N4c5ccccc5B5c6ccccc6N(c6ccccc6)c6cc2c3c4c65)cc1 ; c1ccc2c(c1)B1c3ccccc3N3c4ccccc4B4c5ccccc5N5c6ccccc6B6c7ccccc7N2c2c1c3c4c5c26]

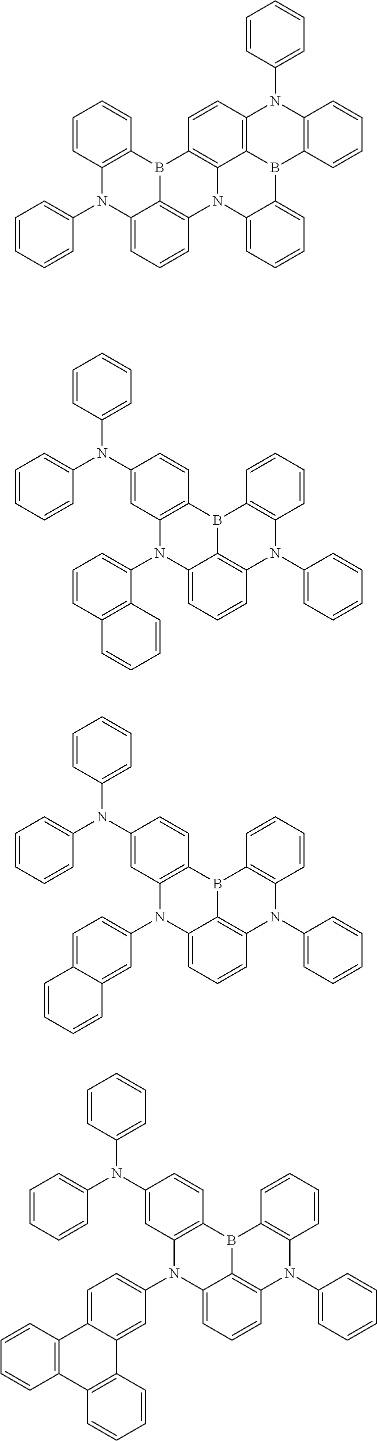 [SMILES: c1ccc(N2c3ccccc3B3c4ccc5c6c4N(c4ccccc4B6c4ccccc4N5c4ccccc4)c4cccc2c43)cc1 ; c1ccc(N(c2ccccc2)c2ccc3c(c2)N(c2cccc4ccccc24)c2cccc4c2B3c2ccccc2N4c2ccccc2)cc1 ; c1ccc(N(c2ccccc2)c2ccc3c(c2)N(c2ccc4ccccc4c2)c2cccc4c2B3c2ccccc2N4c2ccccc2)cc1 ; c1ccc(N(c2ccccc2)c2ccc3c(c2)N(c2ccc4c5ccccc5c5ccccc5c4c2)c2cccc4c2B3c2ccccc2N4c2ccccc2)cc1]

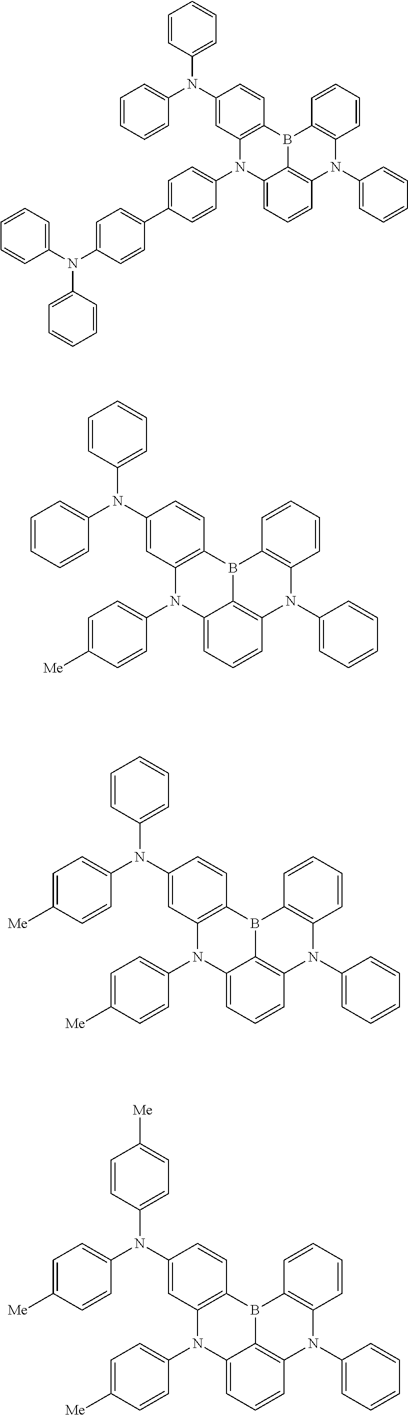 [SMILES: c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1 ; Cc1ccc(N2c3cc(N(c4ccccc4)c4ccccc4)ccc3B3c4ccccc4N(c4ccccc4)c4cccc2c43)cc1 ; Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1]

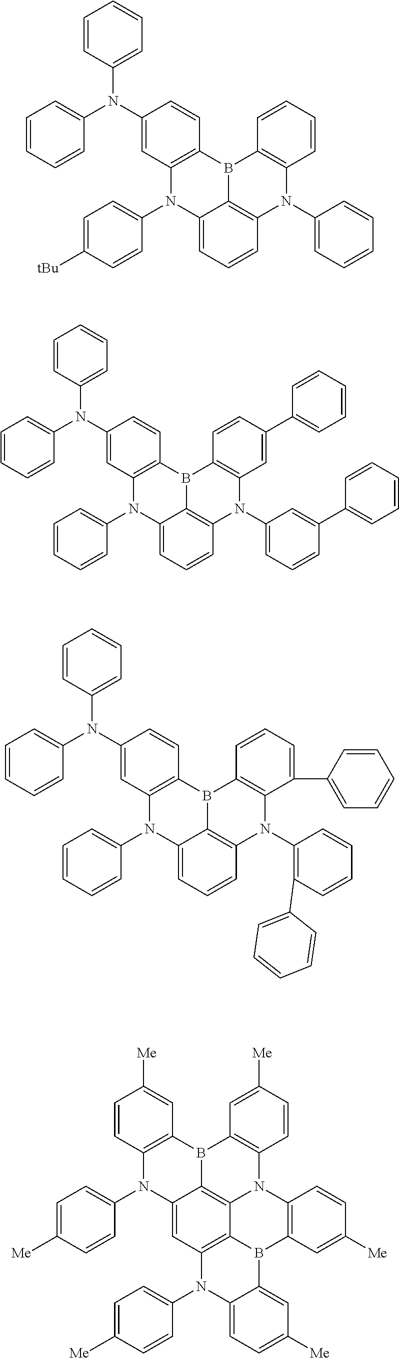 [SMILES: CC(C)(C)c1ccc(N2c3cc(N(c4ccccc4)c4ccccc4)ccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1 ; Cc1ccc(N2c3ccc(C)cc3B3c4cc(C)ccc4N4c5ccc(C)cc5B5c6cc(C)ccc6N(c6ccc(C)cc6)c6cc2c3c4c65)cc1]

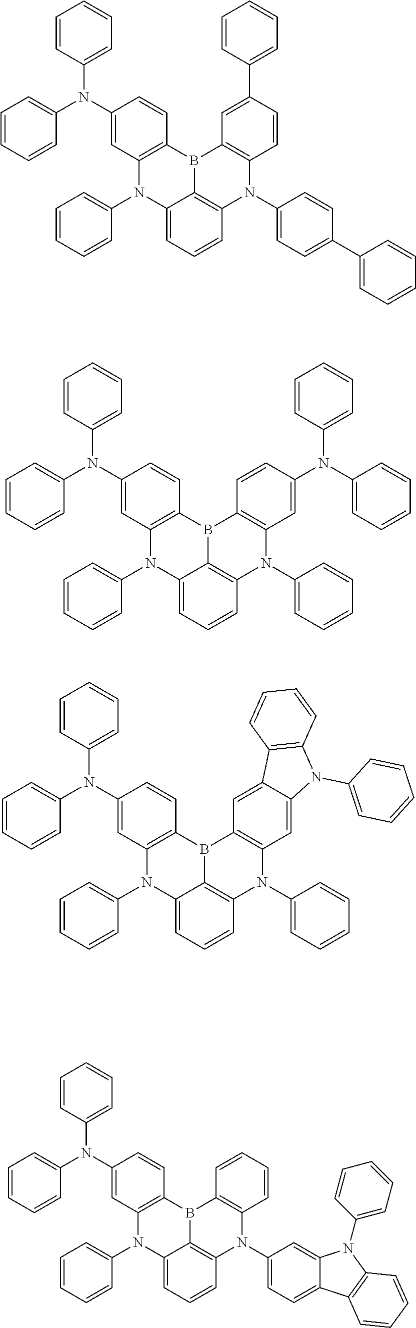 [SMILES: c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cccc4c2B3c2cc3c5ccccc5n(-c5ccccc5)c3cc2N4c2ccccc2)cc1 ; c1ccc(N(c2ccccc2)c2ccc3c(c2)N(c2ccccc2)c2cccc4c2B3c2ccccc2N4c2ccc3c4ccccc4n(-c4ccccc4)c3c2)cc1]

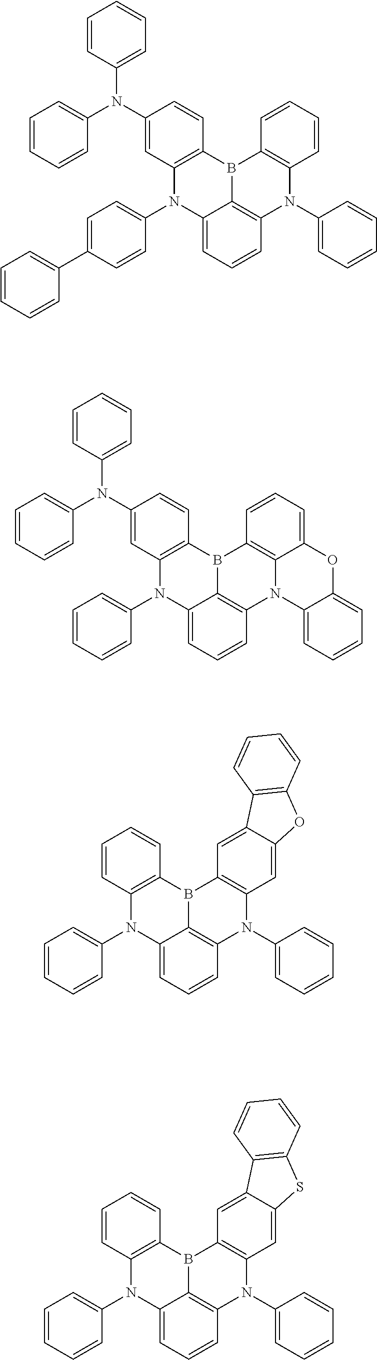 [SMILES: c1ccc(-c2ccc(N3c4cc(N(c5ccccc5)c5ccccc5)ccc4B4c5ccccc5N(c5ccccc5)c5cccc3c54)cc2)cc1 ; c1ccc(N(c2ccccc2)c2ccc3c(c2)N(c2ccccc2)c2cccc4c2B3c2cccc3c2N4c2ccccc2O3)cc1 ; c1ccc(N2c3ccccc3B3c4cc5c(cc4N(c4ccccc4)c4cccc2c43)oc2ccccc25)cc1 ; c1ccc(N2c3ccccc3B3c4cc5c(cc4N(c4ccccc4)c4cccc2c43)sc2ccccc25)cc1]

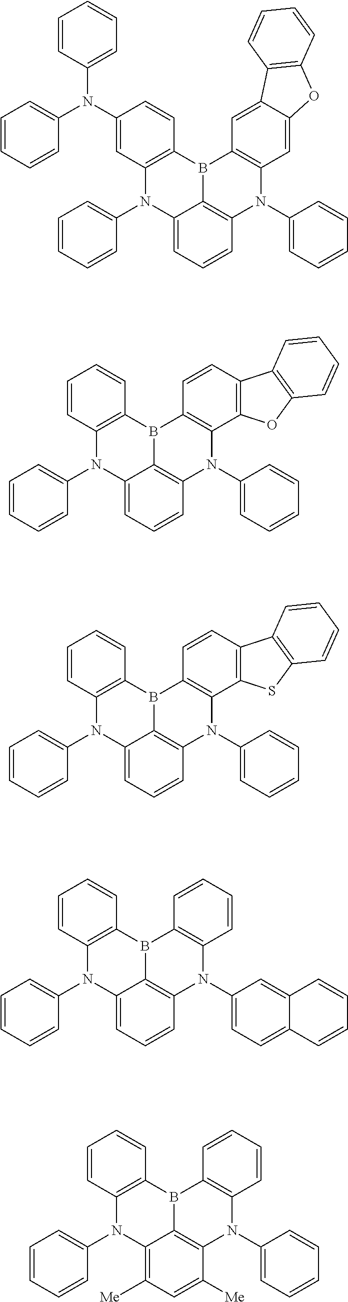 [SMILES: c1ccc(N(c2ccccc2)c2ccc3c(c2)N(c2ccccc2)c2cccc4c2B3c2cc3c(cc2N4c2ccccc2)oc2ccccc23)cc1 ; c1ccc(N2c3ccccc3B3c4ccc5c(oc6ccccc65)c4N(c4ccccc4)c4cccc2c43)cc1 ; c1ccc(N2c3ccccc3B3c4ccc5c(sc6ccccc65)c4N(c4ccccc4)c4cccc2c43)cc1 ; c1ccc(N2c3ccccc3B3c4ccccc4N(c4ccc5ccccc5c4)c4cccc2c43)cc1 ; Cc1cc(C)c2c3c1N(c1ccccc1)c1ccccc1B3c1ccccc1N2c1ccccc1]

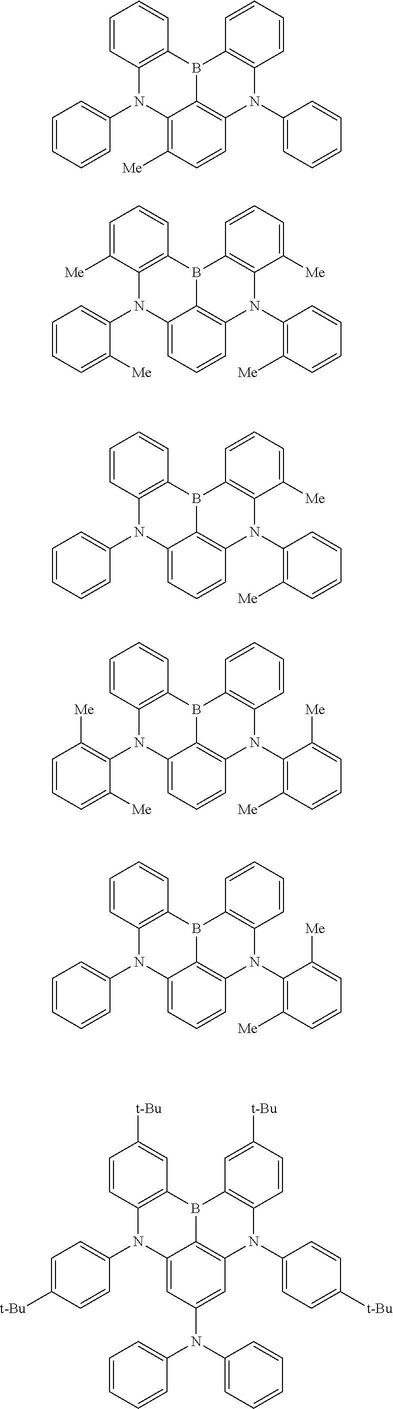

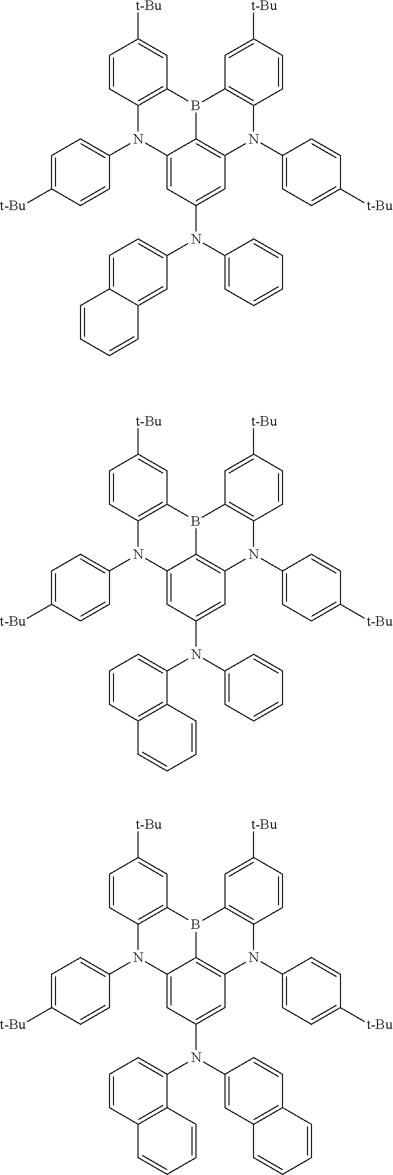 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccc6ccccc6c5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5cccc6ccccc56)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5cccc6ccccc56)cc2c43)cc1]

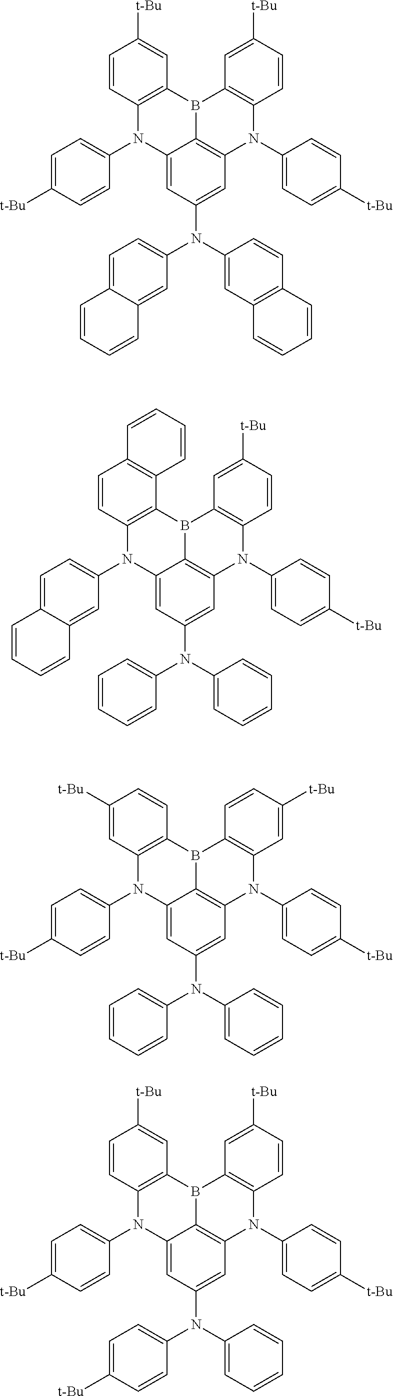

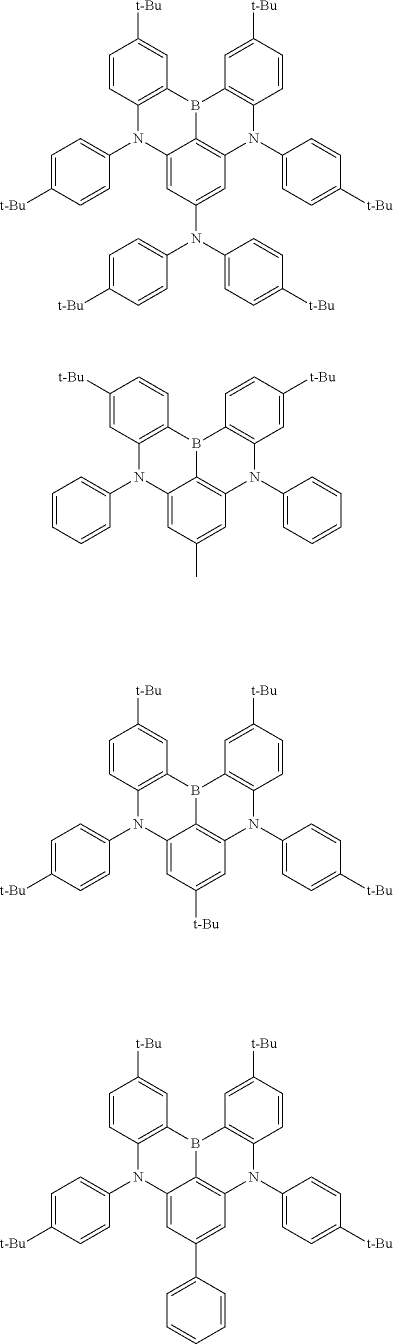 [SMILES: CC(C)(C)c1ccc(N(c2ccc(C(C)(C)C)cc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1 ; Cc1cc2c3c(c1)N(c1ccccc1)c1cc(C(C)(C)C)ccc1B3c1ccc(C(C)(C)C)cc1N2c1ccccc1 ; CC(C)(C)c1ccc(N2c3ccc(C(C)(C)C)cc3B3c4cc(C(C)(C)C)ccc4N(c4ccc(C(C)(C)C)cc4)c4cc(C(C)(C)C)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccccc5)cc2c43)cc1]

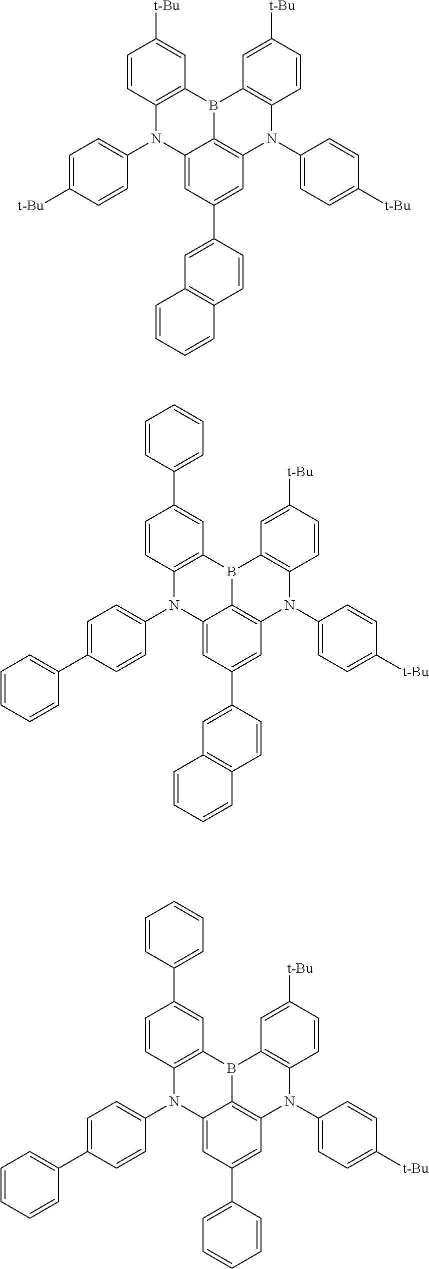 [SMILES: CC(C)(C)c1ccc(N2c3ccc(C(C)(C)C)cc3B3c4cc(C(C)(C)C)ccc4N(c4ccc(C(C)(C)C)cc4)c4cc(-c5ccc6ccccc6c5)cc2c43)cc1 ; CC(C)(C)c1ccc(N2c3ccc(C(C)(C)C)cc3B3c4cc(-c5ccccc5)ccc4N(c4ccc(-c5ccccc5)cc4)c4cc(-c5ccc6ccccc6c5)cc2c43)cc1 ; CC(C)(C)c1ccc(N2c3ccc(C(C)(C)C)cc3B3c4cc(-c5ccccc5)ccc4N(c4ccc(-c5ccccc5)cc4)c4cc(-c5ccccc5)cc2c43)cc1]

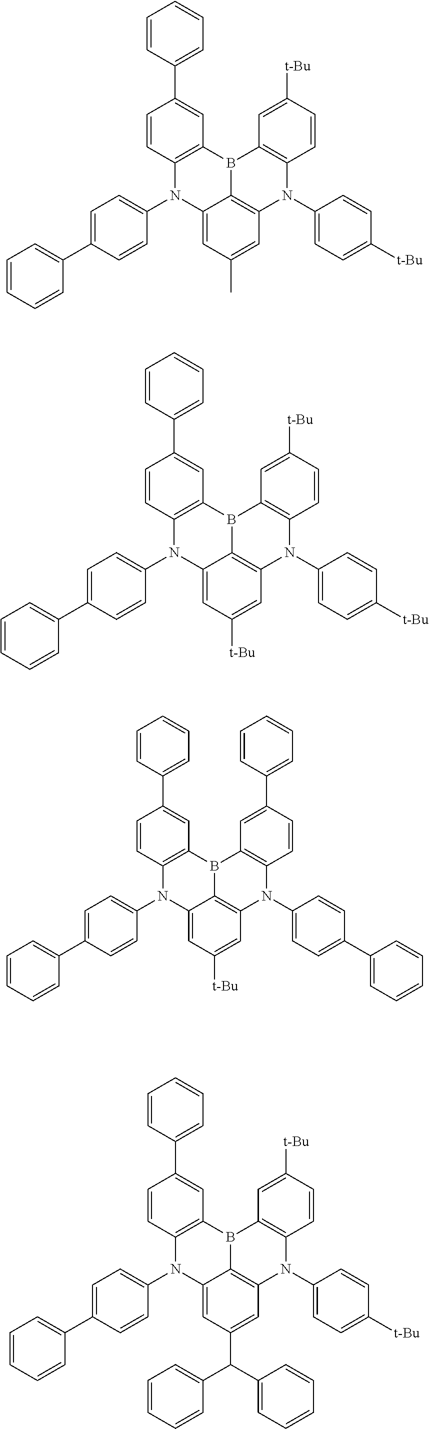 [SMILES: Cc1cc2c3c(c1)N(c1ccc(C(C)(C)C)cc1)c1ccc(C(C)(C)C)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(C(C)(C)C)cc2c43)cc1 ; CC(C)(C)c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(C(c5ccccc5)c5ccccc5)cc2c43)cc1]

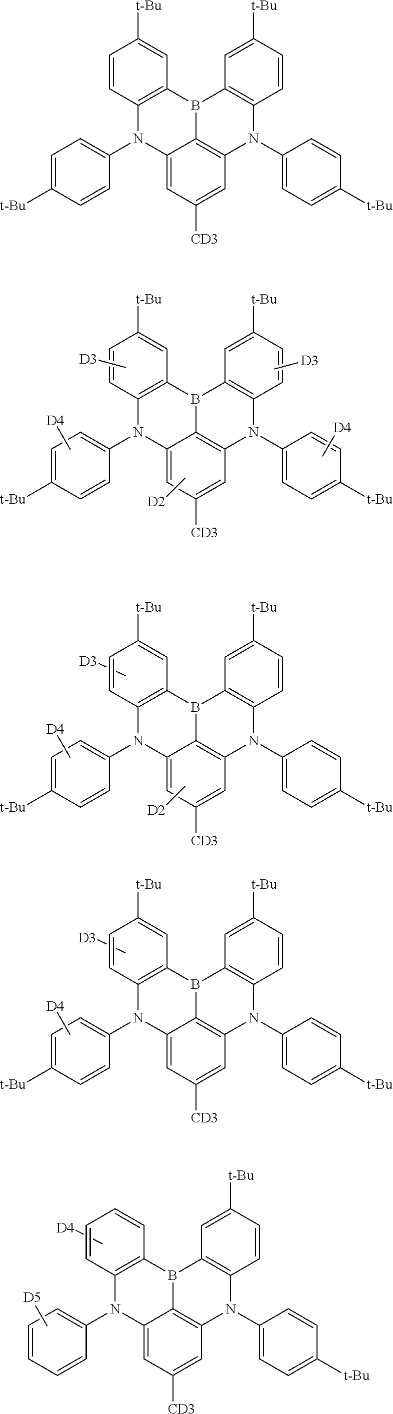

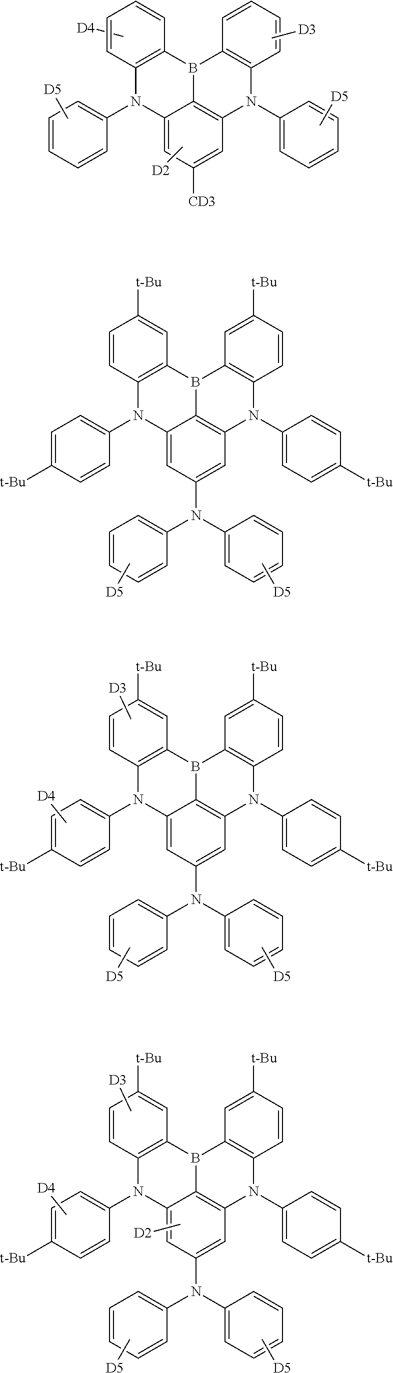

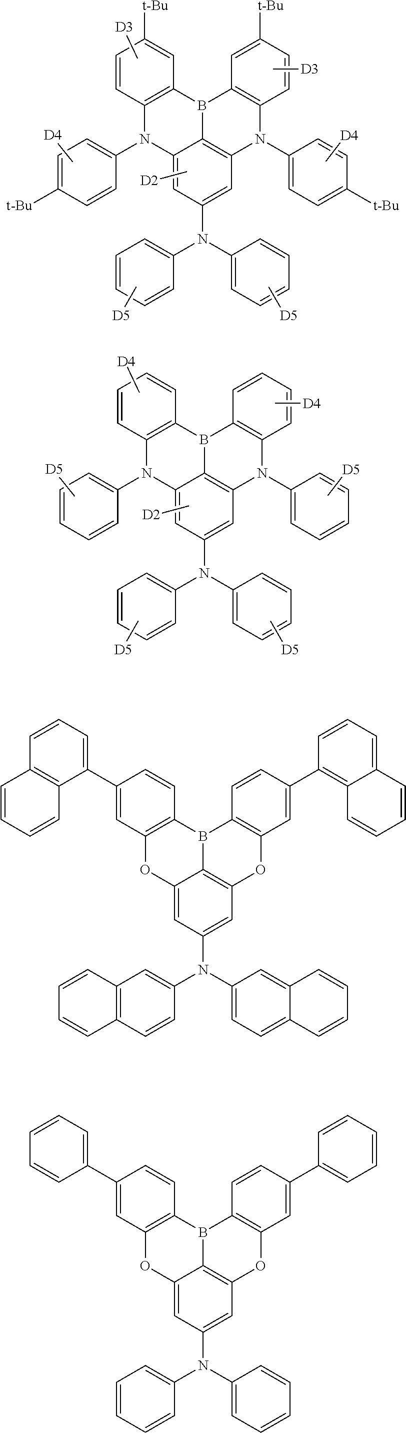

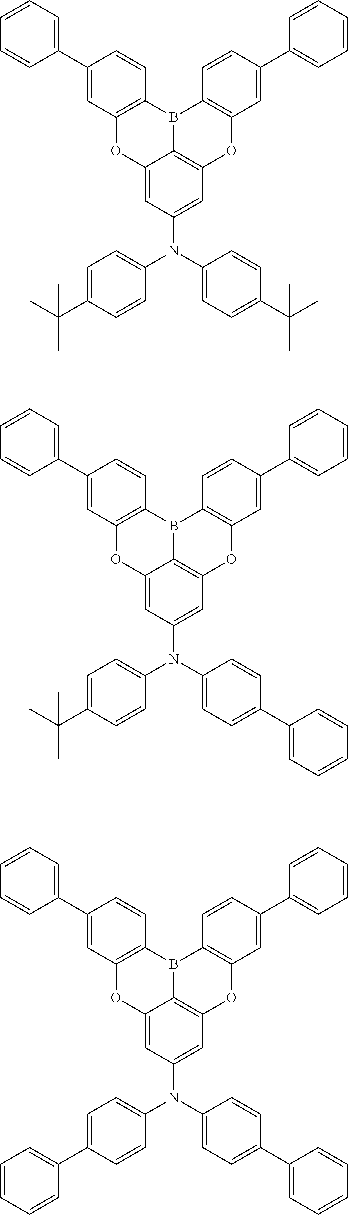 [SMILES: CC(C)(C)c1ccc(N(c2ccc(C(C)(C)C)cc2)c2cc3c4c(c2)Oc2cc(-c5ccccc5)ccc2B4c2ccc(-c4ccccc4)cc2O3)cc1 ; CC(C)(C)c1ccc(N(c2ccc(-c3ccccc3)cc2)c2cc3c4c(c2)Oc2cc(-c5ccccc5)ccc2B4c2ccc(-c4ccccc4)cc2O3)cc1 ; c1ccc(-c2ccc(N(c3ccc(-c4ccccc4)cc3)c3cc4c5c(c3)Oc3cc(-c6ccccc6)ccc3B5c3ccc(-c5ccccc5)cc3O4)cc2)cc1]

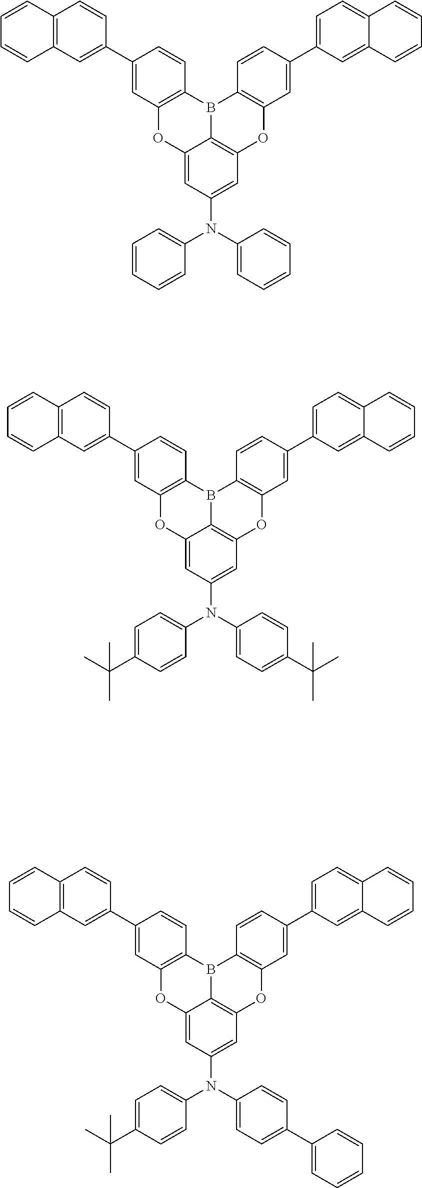 [SMILES: c1ccc(N(c2ccccc2)c2cc3c4c(c2)Oc2cc(-c5ccc6ccccc6c5)ccc2B4c2ccc(-c4ccc5ccccc5c4)cc2O3)cc1 ; CC(C)(C)c1ccc(N(c2ccc(C(C)(C)C)cc2)c2cc3c4c(c2)Oc2cc(-c5ccc6ccccc6c5)ccc2B4c2ccc(-c4ccc5ccccc5c4)cc2O3)cc1 ; CC(C)(C)c1ccc(N(c2ccc(-c3ccccc3)cc2)c2cc3c4c(c2)Oc2cc(-c5ccc6ccccc6c5)ccc2B4c2ccc(-c4ccc5ccccc5c4)cc2O3)cc1]

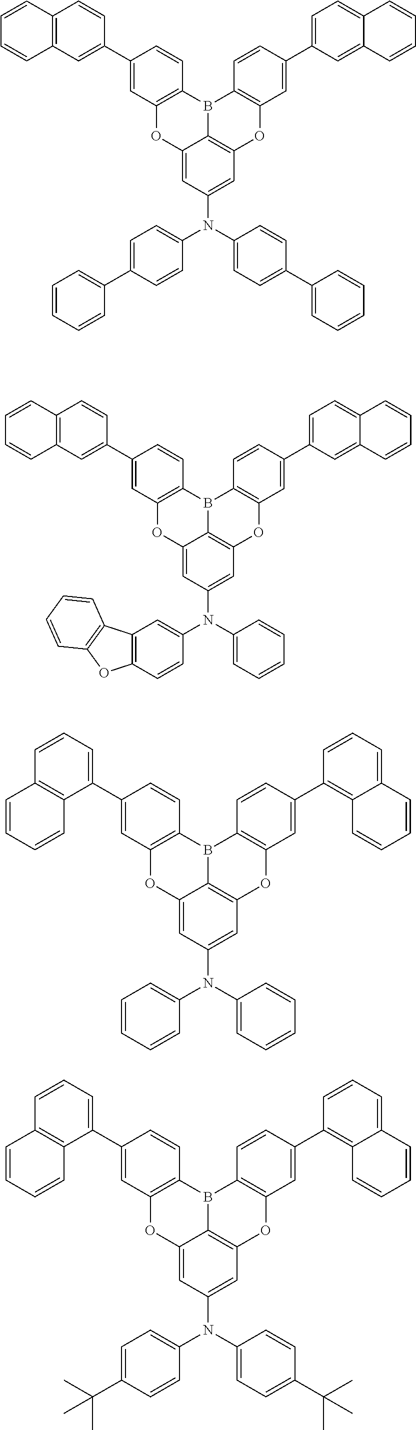 [SMILES: c1ccc(-c2ccc(N(c3ccc(-c4ccccc4)cc3)c3cc4c5c(c3)Oc3cc(-c6ccc7ccccc7c6)ccc3B5c3ccc(-c5ccc6ccccc6c5)cc3O4)cc2)cc1 ; c1ccc(N(c2cc3c4c(c2)Oc2cc(-c5ccc6ccccc6c5)ccc2B4c2ccc(-c4ccc5ccccc5c4)cc2O3)c2ccc3oc4ccccc4c3c2)cc1 ; c1ccc(N(c2ccccc2)c2cc3c4c(c2)Oc2cc(-c5cccc6ccccc56)ccc2B4c2ccc(-c4cccc5ccccc45)cc2O3)cc1 ; CC(C)(C)c1ccc(N(c2ccc(C(C)(C)C)cc2)c2cc3c4c(c2)Oc2cc(-c5cccc6ccccc56)ccc2B4c2ccc(-c4cccc5ccccc45)cc2O3)cc1]

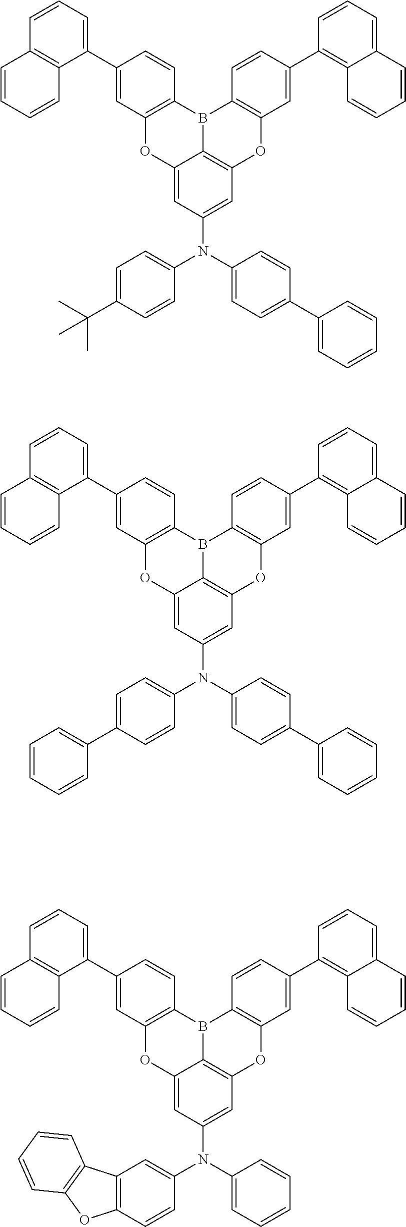 [SMILES: CC(C)(C)c1ccc(N(c2ccc(-c3ccccc3)cc2)c2cc3c4c(c2)Oc2cc(-c5cccc6ccccc56)ccc2B4c2ccc(-c4cccc5ccccc45)cc2O3)cc1 ; c1ccc(-c2ccc(N(c3ccc(-c4ccccc4)cc3)c3cc4c5c(c3)Oc3cc(-c6cccc7ccccc67)ccc3B5c3ccc(-c5cccc6ccccc56)cc3O4)cc2)cc1 ; c1ccc(N(c2cc3c4c(c2)Oc2cc(-c5cccc6ccccc56)ccc2B4c2ccc(-c4cccc5ccccc45)cc2O3)c2ccc3oc4ccccc4c3c2)cc1]

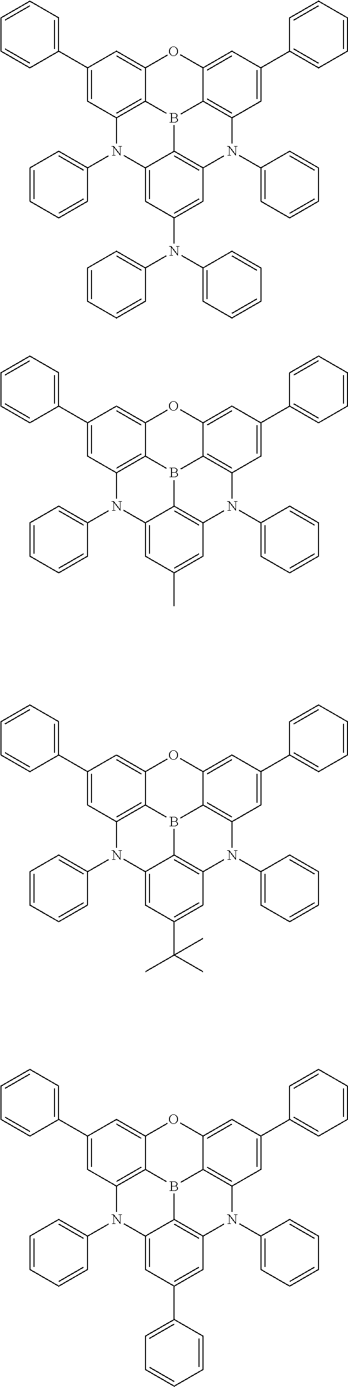 [SMILES: c1ccc(-c2cc3c4c(c2)N(c2ccccc2)c2cc(N(c5ccccc5)c5ccccc5)cc5c2B4c2c(cc(-c4ccccc4)cc2N5c2ccccc2)O3)cc1 ; Cc1cc2c3c(c1)N(c1ccccc1)c1cc(-c4ccccc4)cc4c1B3c1c(cc(-c3ccccc3)cc1N2c1ccccc1)O4 ; CC(C)(C)c1cc2c3c(c1)N(c1ccccc1)c1cc(-c4ccccc4)cc4c1B3c1c(cc(-c3ccccc3)cc1N2c1ccccc1)O4 ; c1ccc(-c2cc3c4c(c2)N(c2ccccc2)c2cc(-c5ccccc5)cc5c2B4c2c(cc(-c4ccccc4)cc2N5c2ccccc2)O3)cc1]

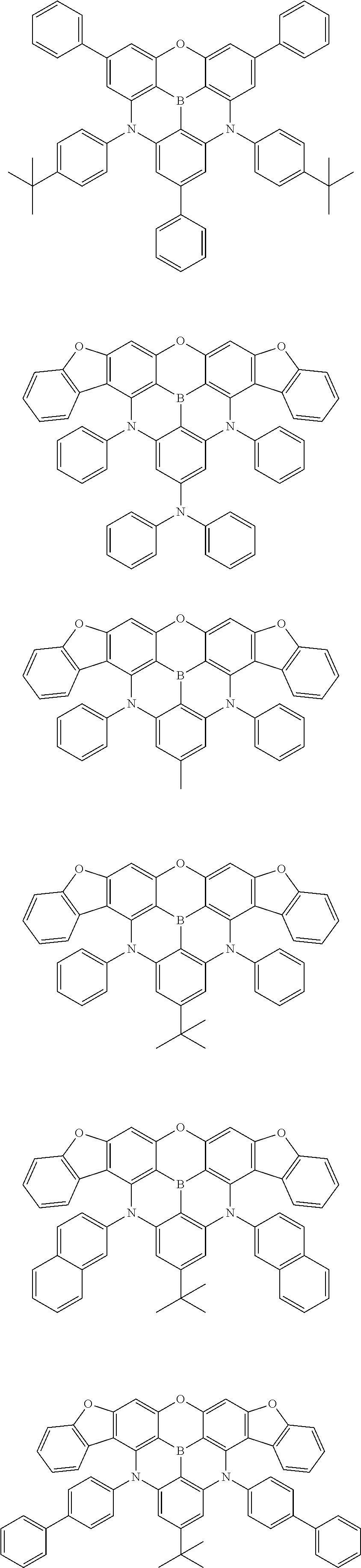

In the compounds above, D2 to D5 respectively represent that 2 to 5 hydrogens are replaced with deuterium. For example, D5 represents that 5 hydrogens are replaced with deuterium.

The plurality of organic electroluminescent materials according to one embodiment of the present disclosure may comprise the compound represented by formula 1 of the present disclosure as a hole transport zone material, and the compound represented by formula 2 of the present disclosure as a dopant material. The hole transport zone material may be at least one selected from the group consisting of a hole transport material, a hole injection material, an electron blocking material, and a hole auxiliary material.

The compound represented by formula 1 according to the present disclosure may be prepared by a synthetic method known to one skilled in the art. For example, the compound represented by formula 1 can be prepared by referring to Japan Patent No. 3915256 B2 (published on Feb. 16, 2007), Korean Patent No. 0817380 B1 (published on Mar. 20, 2008), Japan Patent No. 4377783 B2 (published on Sep. 18, 2009), U.S. Patent Application Publication No. 201710025609 A1 (published on Jan. 26, 2017), and Korean Patent Application Laid-Open No. 2017-0080432 A (published on Jul. 10, 2017), but are not limited thereto.

The compound represented by formula 2 according to the present disclosure may be prepared by a synthetic method known to one skilled in the art. For example, the compound represented by formula 2 can be prepared by referring to Korean Patent No. 1876763 B1 (published on Jul. 11, 2018), Japan Patent No. 5935199 B2 (published on May 20, 2016), and Korean Patent Appl. Laid-Open No. 2017-0130434 A (published on Nov. 28, 2017), but are not limited thereto.

The deuterated compound of the present disclosure may be prepared in a similar manner by using deuterated precursor materials, or more generally may be prepared by treating the non-deuterated compound with a deuterated solvent or D6-benzene in the presence of an H/D exchange catalyst such as a Lewis acid, e.g., aluminum trichloride or ethyl aluminum chloride. In addition, the degree of deuteration can be controlled by changing the reaction conditions such as the reaction temperature. For example, the number of deuterium in the formula of the present disclosure can be controlled by adjusting the reaction temperature, time, the equivalent of the acid, etc.

The organic electroluminescent device according to the present disclosure comprises a first electrode, a second electrode facing the first electrode, and an organic layer(s) between the first and second electrodes. The organic layer comprises at least one layer of hole transport zone and at least one light-emitting layer; at least one layer of the hole transport zone comprises the compound represented by formula 1; at least one layer of the light-emitting layer comprises at least one dopant compound and at least one host compound; and the at least one dopant compound comprises the compound represented by formula 2.

The organic layer may further comprise at least one layer selected from a light-emitting auxiliary layer, an electron transport layer, an electron buffer layer, an electron injection layer, an interlayer and a hole blocking layer, in addition to the light-emitting layer and the hole transport zone.

The hole transport zone of the present disclosure may be composed of one or more layers selected from the group consisting of a hole transport layer, a hole injection layer, an electron blocking layer, and a hole auxiliary layer, and each of the layers may be composed of one or more layers, Preferably, the hole transport zone may comprise a hole transport layer. In addition, the hole transport zone comprises a hole transport layer, and may further comprise at least one of a hole injection layer, a electron blocking layer and a hole auxiliary layer,

Herein, the hole auxiliary layer or the light-emitting auxiliary layer is placed between the hole transport layer and the light-emitting layer, and controls a hole transport rate. The hole auxiliary layer or the light-emitting auxiliary layer provides an effect of improving the efficiency and lifetime of the organic electroluminescent device.

According to one embodiment of the present disclosure, the hole transport layer may be composed of a single layer, and may include a hole transport material comprising the compound represented by formula 1 of the present disclosure.

According to another embodiment of the present disclosure, the hole transport zone comprises a hole transport layer, and the hole transport layer may consist of multi-layers of two or more layers, wherein at least one layer of the multi-layers may comprise the hole transport material comprising the compound represented by formula 1 of the present disclosure. The hole transport layer comprising the compound represented by formula 1 or other layers may comprise all compounds used in conventional hole transport materials. For example, a compound represented by the following formula 10 may be comprised.

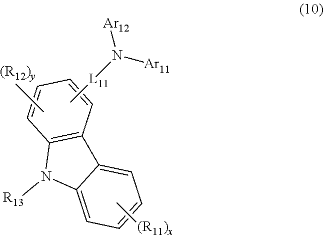

In formula 10,

L₁₁ represents a single bond, a substituted or unsubstituted (C6-C30)arylene, or a substituted or unsubstituted (3- to 30-membered)heteroarylene;

Ar₁₁ and Ar₁₂, each independently, represent a substituted or unsubstituted (C6-C30)aryl, or a substituted or unsubstituted (3- to 30-membered)heteroaryl, or Ar₁₁ and may form a nitrogen-containing (3- to 30-membered)heteroaryl with the nitrogen to which they are bound;

R₁₁ to R₁₃, each independently, represent hydrogen, deuterium, a halogen, a cyano, a carboxyl, a nitro, a hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C1-C30)alkoxy, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₄₁R₄₂, —SiR₄₃R₄₄R₄₅, —SR₄₆, —OR₄₇, —COR₄₈, or —B(OR₄₉)(OR₅₀), or are linked to an adjacent substituent(s) to form a mono- or polycyclic (3- to 30-membered) alicyclic or aromatic ring, or the combination thereof, whose carbon atom(s) may be replaced with at least one heteroatom selected from nitrogen, oxygen, and sulfur;

R₄₁ to R₅₀, each independently, represent hydrogen, deuterium, a halogen, a cyano, a carboxyl, a nitro, a hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C1-C30)alkoxy, a substituted or unsubstituted (C₃-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or are linked to an adjacent substituent(s) to form a mono- or polycyclic (3- to 30-membered) alicyclic or aromatic ring, or the combination thereof, whose carbon atom(s) may be replaced with at least one heteroatom selected from nitrogen, oxygen, and sulfur;

x represents an integer of 1 to 4, in which if x is an integer of 2 or more, each of R₁₁ may be the same or different;

y represents an integer of 1 to 3, in which if y is an integer of 2 or more, each of R₁₂ may be the same or different;

the heteroaryl(ene) contains at least one heteroatom selected from B, N, O, S, Si, and P; and

the heterocycloalkyl contains at least one heteroatom selected from O, S, and N.

The compound represented by formula 2 of the present disclosure may be comprised in the light-emitting layer, When used in the light-emitting layer, the organic electroluminescent compound represented by formula 2 of the present disclosure may be included as a dopant material. Preferably, the light-emitting layer may further comprise one or more host materials.

The host material may be any known hosts, preferably any known fluorescent hosts, but is more preferably one selected from the following formula 11.

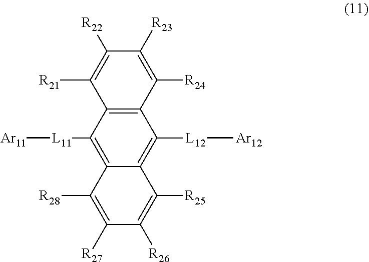

In formula 11,

L₁₁ and L₁₂, each independently, represent a single bond, a substituted or unsubstituted (C6-C30)arylene, or a substituted or unsubstituted (3- to 30-membered)heteroarylene;

Ar₁₁ and Ar₁₂, each independently, represent a substituted or unsubstituted (C6-C30)aryl, or a substituted or unsubstituted (3- to 30-membered)heteroaryl, and

R₂₁ to R₂₈, each independently, represent hydrogen, deuterium, a halogen, a cyano, a substituted or unsubstituted (C1-C30)alkyl, a substituted or unsubstituted (C6-C30)aryl, or a substituted or unsubstituted (3- to 30-membered)heteroaryl, a substituted or unsubstituted (C₃-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (3- to 30-membered)heteroaryl(C6-C30)arylamino.

The light-emitting layer is a layer comprising a host(s) and a dopant(s) from which light is emitted, and may be a single layer or a multi-layer in which two or more layers are stacked. Here, the host mainly has a function of promoting recombination of electrons and holes and confining excitons in the light-emitting layer, and the dopant has a function of allowing excitons obtained by recombination to efficiently emit light. The dopant compound of the light-emitting layer may be doped to less than 25% by weight, preferably less than 17% by weight based on the total amount of the host and dopant compounds.

One of the first and second electrodes may be an anode, and the other may be a cathode. The second electrode may be a transflective electrode or a reflective electrode, and may be a top emission type, a bottom emission type, or a both-sides emission type, depending on the materials.

The organic layer may further comprise an amine-based compound and/or an azine-based compound in addition to the light-emitting material(s) of the present disclosure. Specifically, the hole injection layer, the hole transport layer, the hole auxiliary layer, the light-emitting layer, the light-emitting auxiliary layer, and/or the electron blocking layer may comprise an amine-based compound, e.g., an arylamine-based compound, a styrylarylamine-based compound, etc., as a hole injection material, a hole transport material, a hole auxiliary material, a light-emitting material, a light-emitting auxiliary material, and/or an electron blocking material. In addition, the electron transport layer, the electron injection layer, the electron buffer layer, and/or the hole blocking layer may comprise an azine-based compound as an electron transport material, an electron injection material, an electron buffer material, and/or a hole blocking material.

In addition, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal.

A hole injection layer, a hole transport layer, or an electron blocking layer, or a combination thereof may be used between the anode and the light-emitting layer. The hole injection layer may be multilayers in order to lower the hole injection barrier (or hole injection voltage) from the anode to the hole transport layer or the electron blocking layer, wherein each of the multilayers may use two compounds simultaneously. The electron blocking layer may be located between the hole transport layer (or the hole injection layer) and the light-emitting layer, and may block the overflow of electrons from the light-emitting layer to trap the excitons in the light-emitting layer to prevent light leakage. The hole transport layer or the electron blocking layer may be multilayers, wherein each of the multilayers may use a plurality of compounds.

An electron buffer layer, a hole blocking layer, an electron transport layer, or an electron injection layer, or a combination thereof may be used between the light-emitting layer and the cathode. The electron buffer layer may be multilayers in order to control the injection of the electrons and improve the interfacial properties between the light-emitting layer and the electron injection layer, wherein each of the multilayers may use two compounds simultaneously. The hole blocking layer or the electron transport layer may also be multilayers, wherein each of the multilayers may use a plurality of compounds.

A light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes.

In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In the organic electroluminescent device of the present disclosure, at least one layer selected from a chalcogenide layer, a metal halide layer and a metal oxide layer (hereinafter, “a surface layer”) may be preferably placed on an inner surface(s) of one or both electrodes. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer may provide operation stability for the organic electroluminescent device. Preferably, the chalcogenide includes SiOx (1≤X≤2), AlOx (1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

In the organic electroluminescent device of the present disclosure, a mixed region of an electron transport compound and a reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be used as a charge-generating layer to prepare an organic electroluminescent device having two or more light-emitting layers and emitting white light.

Various structures have been proposed for the white organic electroluminescent device, for example, a side-by-side structure or a stacking structure depending on the arrangement of R (red), G (green) or YG (yellow green), and B (blue) light emitting parts, or a color conversion material (CCM) method, etc. The plurality of organic electroluminescent materials of the present disclosure may also be applied to such white organic electroluminescent device.

The plurality of organic electroluminescent materials according to one embodiment of the present disclosure may also be applied to the organic electroluminescent device comprising a QIP (quantum dot).

In addition, the present disclosure may provide a display system by using the plurality of organic electroluminescent materials according to one embodiment of the present disclosure. That is, it is possible to produce a display system or a lighting system by using the compounds of the present disclosure. Specifically, it is possible to produce a display system, e.g., a display system for smartphones, tablets, notebooks, PCs, TVs, or cars, or a lighting system, e.g., an outdoor or indoor lighting system, by using the compounds of the present disclosure.

In order to form each layer of the organic electroluminescent device of the present disclosure, dry film-forming methods such as vacuum evaporation, sputtering, plasma and ion plating methods, or wet film-forming methods such as ink jet printing, nozzle printing, slot coating, spin coating, dip coating, and flow coating methods may be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

The host and dopant compounds of the present disclosure may be co-evaporated or mixture-evaporated. The co-evaporation is a mixed deposition method in which two or more isomer materials are placed in a respective individual crucible source and a current is applied to both cells at the same time to evaporate the materials. The mixture-evaporation is a mixed deposition method in which two or more isomer materials are mixed in one crucible source before evaporating them, and a current is applied to the cell to evaporate the materials.

Hereinafter, the preparation method of the compound of the present disclosure and the properties thereof will be explained in detail with reference to the representative compounds of the present disclosure. However, the present disclosure is not limited by the following examples.

### EXAMPLE 1

### Preparation of Compound C-2

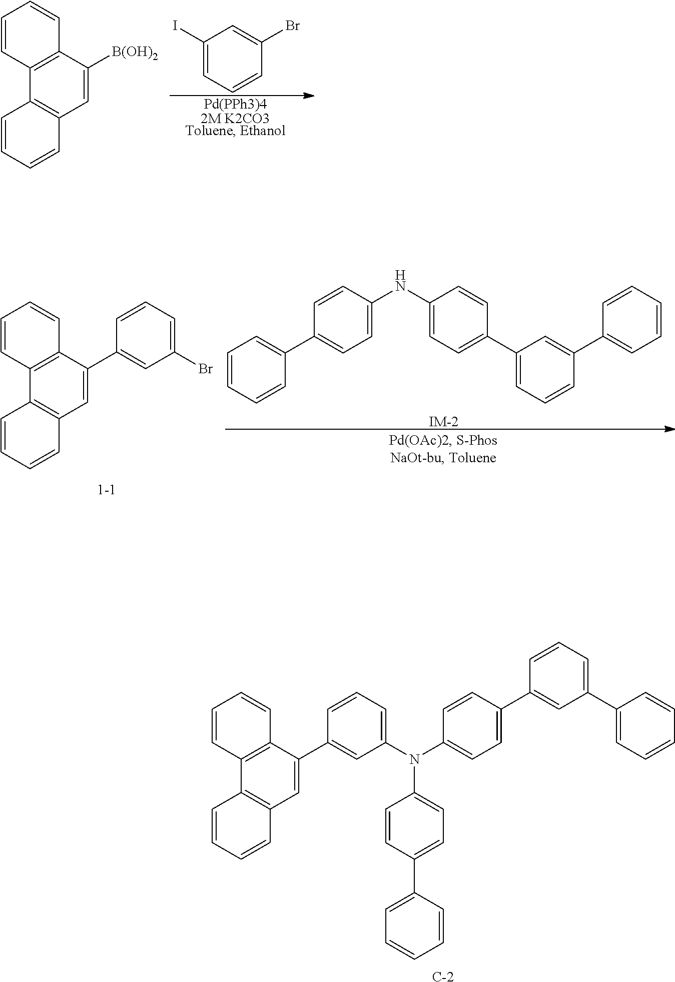

Synthesis of compound 1-1

15 g of 9-phenanthrenyl-boronic acid (0.068 mol), 28.7 g of 1-bromo-3-iodo benzene (0.10 mol), 3.9 g of Pd(PPh₃)₄ (0.003 mol), 18.7 g of K₂CO₃ (0.135 mol), 510 mL of toluene, 65 mL of ethanol, and 65 mL of distilled water were added in a flask, and the mixture was stirred at 100° C. for 5 hours. The reaction mixture was cooled to room temperature, and an organic layer was extracted with ethyl acetate. The residual moisture was removed with magnesium sulfate, and the organic layer was dried, and separated by column chromatography to obtain 13.9 g of compound 1-1 (yield: 61.7%).

Synthesis of compound C-2

40.6 g of compound 1-1 (0.122 mol), 53.3 g of N-([1,1′-biphenyl]-4-yl)-[1,1′:3′,1″-terphenyl]-4-amine (0.134 mol), 1.4 g of Pd(OAc)₂ (0.006 mol), 5 g of S-Phos (0.012 mol), 23.4 g of NaOt-Bu (0.244 mmol), and 610 mL of toluene were added in a flask, and the mixture was stirred under reflux at 150° C. for 2 hours. The reaction mixture was cooled to room temperature, and an organic layer was extracted with ethyl acetate. The residual moisture was removed with magnesium sulfate, and the organic layer was dried, and separated by column chromatography to obtain 54.7 g of compound C-2 (yield: 69.07%).

### EXAMPLE 2

### Preparation of Compound C-50

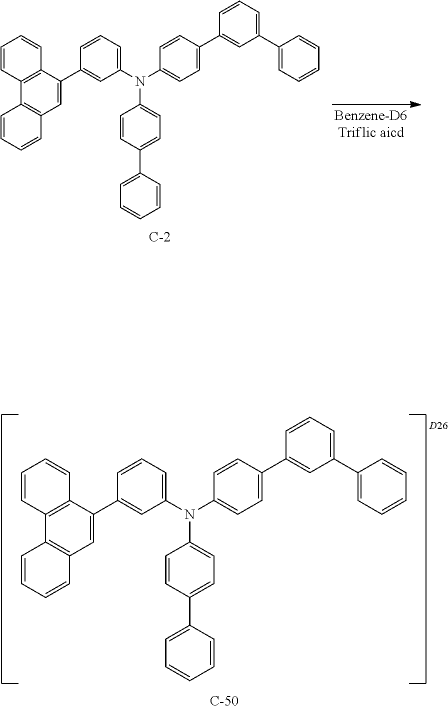

In a flask, 23.0 g of compound C-2 (0.035 mol) were dissolved in 442 mL of benzene-D6, and 18.8 mL of triflic acid (0.212 mol) was added thereto, and then the mixture was stirred at room temperature for 23 hours. 400 mL of 2.0M Na₃PO₄ aqueous solution was slowly added to the reaction mixture. Thereafter, an organic layer was extracted with ethyl acetate. The residual moisture was removed with magnesium sulfate, and the organic layer was dried, and separated by column chromatography to obtain 17.4 g of compound C-50 (yield: 71.9%).

Hereinafter, a method of producing an organic electroluminescent device (OLED) comprising the compound according to the present disclosure and the properties thereof will be explained in detail. However, the following examples merely illustrate the properties of an OLED according to the present disclosure in detail, but the present disclosure is not limited to the following examples.

Device Example 1: Producing an OLEO Using the Compound According to the Present Disclosure

An OLED was produced using the organic electroluminescent compound according to the present disclosure, as follows: A transparent electrode indium tin oxide (ITO) thin film (10 Ω/sq) on a glass substrate for an OLED (GEOMATEC CO., LTD., Japan) was subjected to an ultrasonic washing with acetone, ethanol and distilled water, sequentially, and then was stored in isopropanol. After evacuating until the degree of vacuum in the chamber reached 10⁻⁶ torr, the ITO substrate was mounted on a substrate holder of a vacuum vapor deposition apparatus. Compound HT-1 was introduced into a cell of the vacuum vapor deposition apparatus, and compound HI-1 was introduced into another cell of the vacuum vapor deposition apparatus. The two materials were evaporated, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited on the hole injection layer to form a first hole transport layer having a thickness of 75 nm, Compound C-2 was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows: Compound BH-1 was introduced into a cell of the vacuum vapor deposition apparatus as a host of the light-emitting layer, and compound BD-2 was introduced into another cell as a dopant. The two materials were evaporated, and the dopant was deposited in a doping amount of 2 wt % based on the total amount of the host and dopant to form a light-emitting layer having a thickness of 20 nm on the second hole transport layer. Next, compound ET-1 was deposited to form a hole blocking layer having a thickness of 5 nm on the light-emitting layer. Compound ET-2 and compound EI-1 were evaporated at a rate of 1:1 in two other cells to form an electron transport layer having a thickness of 30 nm on the hole blocking layer. After depositing compound EI-1 as an electron injection layer having a thickness of 2 nm, an Al cathode having a thickness of 80 nm was deposited by another vacuum vapor deposition apparatus. Thus, an OLED was produced.

Device Example 2: Producing an OLED Using the Compound According to the Present Disclosure

An OLED was produced in the same manner as in Device Example 1, except that compound C-50 was used in the second hole transport layer.

Comparative Example 1: Producing an OLED Using a Conventional Compound

An OLED was produced in the same manner as in Device Example 1, except that compound BD-1 was used as the dopant of the light-emitting layer.

Table 1 shows a CIE color coordinate, an electro-luminance wavelength, and a full width at half maximum (FWHM) based on a luminance of 1,000 nit, and the minimum time taken to be reduced from 100% to 95% of the luminance (lifetime; T95) based on a luminance of 2,000 nit of the OLED produced in the Device Examples and the Comparative Example above.

TABLE 1

| | Second | | Electro- | | | | |
|---|---|---|---|---|---|---|---|
| | Hole | | Luminance | | CIE Color | | Lifetime |
| | Transport | | Wavelength | FWHM | Coordinate | | (T95) |
| | Layer | Dopant | [nm] | [nm] | x | y | [hr] |
| Device | C-2 | BD-2 | 457 | 26 | 0.138 | 0.061 | 44 |
| Example 1 | | | | | | | |
| Device | C-50 | BD-2 | 457 | 26 | 0.139 | 0.060 | 59 |
| Example 2 | | | | | | | |
| Comparative | C-2 | BD-1 | 459 | 38 | 0.133 | 0.106 | 26 |
| Example 1 | | | | | | | |

From the Device Examples and the Comparative Example above, it can be confirmed that an organic electroluminescent device comprising a specific combination of the compounds according to the present disclosure as a hole transport material for a hole transport layer and a dopant for a light-emitting layer exhibits deeper blue light-emission compared to a conventional organic electroluminescent device. The deep blue light-emitting OLED can enable a wider color expression in display implementation compared to the conventional blue light-emitting OLED. In addition, it can be confirmed that an organic electroluminescent device comprising a plurality of organic electroluminescent materials according to the present disclosure exhibits longer lifetime properties compared to an organic electroluminescent device using a conventional material. That is, by comprising a plurality of organic electroluminescent materials of the present disclosure, it is possible to provide a deep blue OLED having long lifetime properties.

The compounds used in the Device Examples and the Comparative Example are shown below.

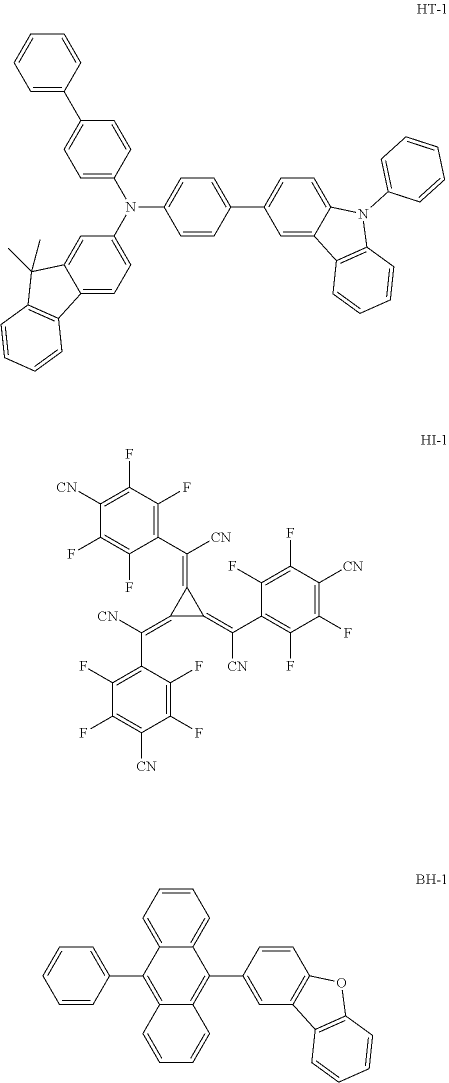

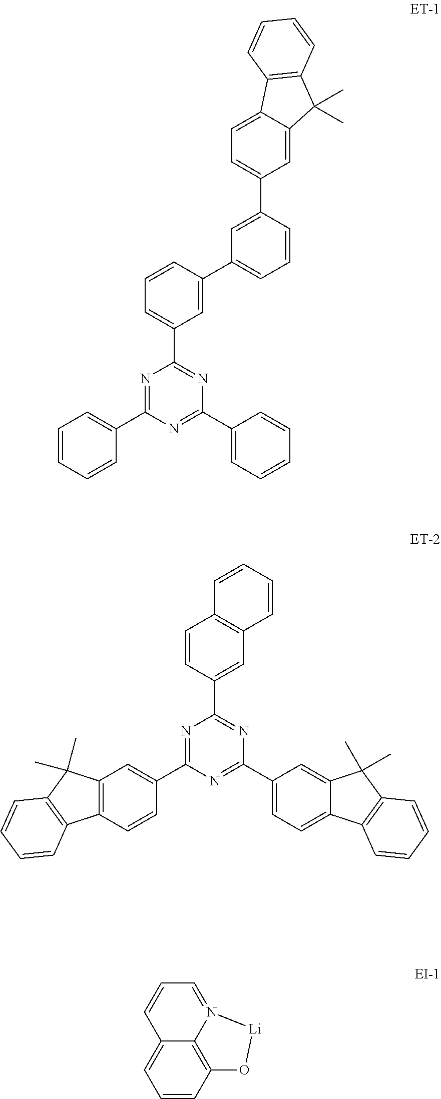

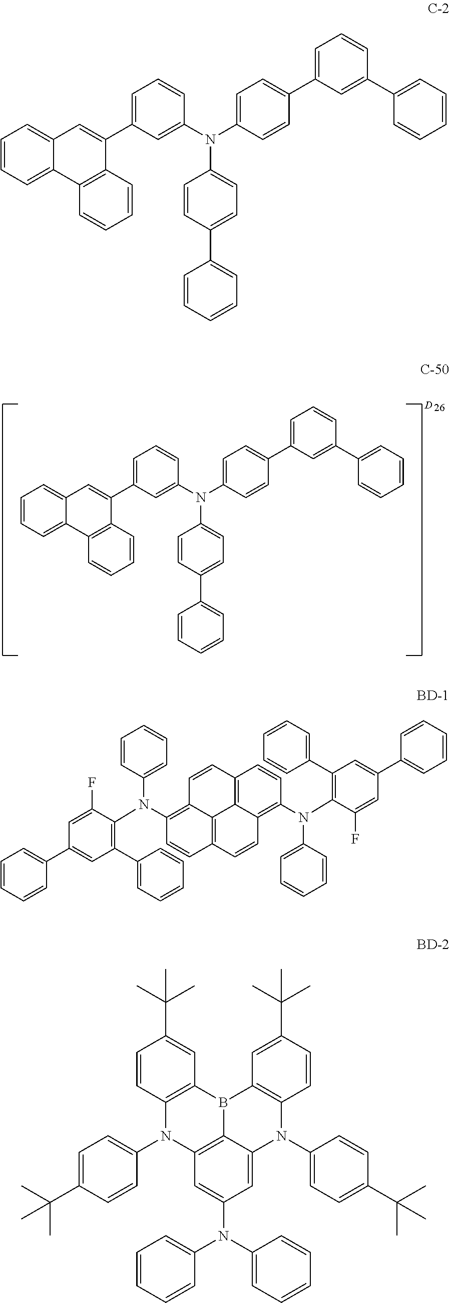

1. A plurality of organic electroluminescent materials comprising at least one first compound and at least one second compound, wherein the first compound is represented by the following formula 1: 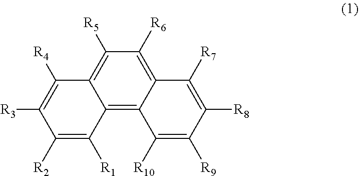
wherein,
R₁ to R₁₀, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or 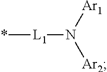
with a proviso that at least one of R₁ to R₁₀ is 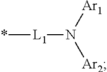
L₁ represents a single bond, a substituted or unsubstituted (C6-C30)arylene, or a substituted or unsubstituted (3- to 30-membered)heteroarylene;
Ar₁ and Ar₂, each independently, represent hydrogen, deuterium, a substituted or unsubstituted (C1-C30)alkyl, a substituted or unsubstituted (C₃-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl, or Ar₁ and Ar₂ may be linked to each other to form a fused ring; and
* represents the position linked to phenanthrene; and
the second compound is represented by the following formula 2: 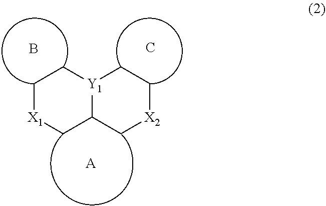
wherein,
ring A, ring B, and ring C, each independently, represent a substituted or unsubstituted mono- or polycyclic (3- to 50-membered) alicyclic or aromatic ring, or the combination thereof, which may contain at least one heteroatom selected from B, N, O, S, Si, and P; and ring B and ring C may be linked to each other to form a fused ring;
Y₁ represents B;
X₁ and X₂, each independently, represent NR or O; and
R, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (3- to 30-membered)heteroaryl(C6-C30)arylamino; or may be linked to at least one of ring A, ring B, and ring C to form a fused ring.

2. The plurality of organic electroluminescent materials according to claim 1, wherein the substituents of the substituted alkyl, the substituted aryl(ene), the substituted heteroaryl(ene), the substituted cycloalkyl, the substituted heterocycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-alkenylamino, the substituted mono- or di-arylamino, the substituted mono- or di-heteroarylamino, the substituted alkylalkenylamino, the substituted alkylarylamino, the substituted alkylheteroarylamino, the substituted alkenylarylamino, the substituted alkenylheteroarylamino, the substituted heteroarylarylamino, and the substituted ring, each independently, are at least one selected from the group consisting of deuterium;
a halogen; a cyano; a carboxyl; a nitro; a hydroxyl; a (C1-C30)alkyl unsubstituted or substituted with at least one deuterium; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl, a (C6-C30)aryloxy; a (C6-C30)arylthio; a (3- to 30-membered)heteroaryl unsubstituted or substituted with at least one of deuterium(s), a (C1-C30)alkyl(s), a (C6-C30)aryl(s), and a di(C6-C30)arylamino(s); a (C6-C30)aryl unsubstituted or substituted with at least one of deuterium(s), a (C1-C30)alkyl(s), a (3- to 30-membered)heteroaryl(s), and a di(C6-C30)arylamino(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino unsubstituted or substituted with at least one of deuterium(s), a (C1-C30)alkyl(s), a (3- to 30-membered)heteroaryl(s), and a di(C6-C30)arylamino(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (3- to 30-membered)heteroaryl(C6-C30)arylamino unsubstituted or substituted with at least one of a (C1-C30)alkyl(s) and a (C6-C30)aryl(s); a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of organic electroluminescent materials according to claim 1, wherein formula 1 is represented by the following formula 1-1: 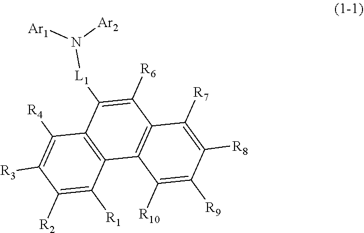
wherein, L₁, Ar₁, Ar₂, R₁ to R₄, and R₆ to R₁₀ are as defined in claim 1.

4. The plurality of organic electroluminescent materials according to claim 1, wherein R₁ to R₁₀, each independently, represent hydrogen, deuterium, a substituted or unsubstituted (C1-C10)alkyl, a substituted or unsubstituted (C6-C25)aryl, a substituted or substituted (5- to 25-membered)heteroaryl, or 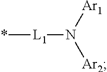
with a proviso that at least one of R₁ to R₁₀ is 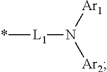
and * represents the position linked to phenanthrene.

5. The plurality of organic electroluminescent materials according to claim 1, wherein R₁ to R₁₀, each independently, represent hydrogen, deuterium, or 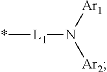
with a proviso that at least one of R₁ to R₁₀ is 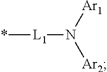
and * represents the position linked to phenanthrene.

6. The plurality of organic electroluminescent materials according to claim 1, wherein formula 2 is represented by the following formula 2-1: 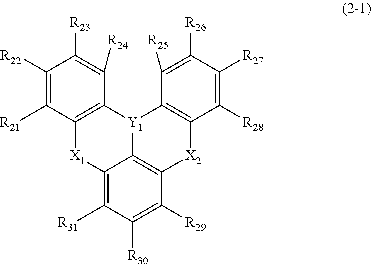
wherein,
Y₁, X₁, and X₂, each independently, are as defined in claim 1; and
R₂₁ to R₃₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)alysilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (3- to 30-membered)heteroaryl(C6-C30)arylamino; or adjacent ones of R₂₁ to R₃ may be linked to each other to form a ring(s).

7. The plurality of organic electroluminescent materials according to claim 1, wherein the compound represented by formula 1 is at least one selected from the following compounds: 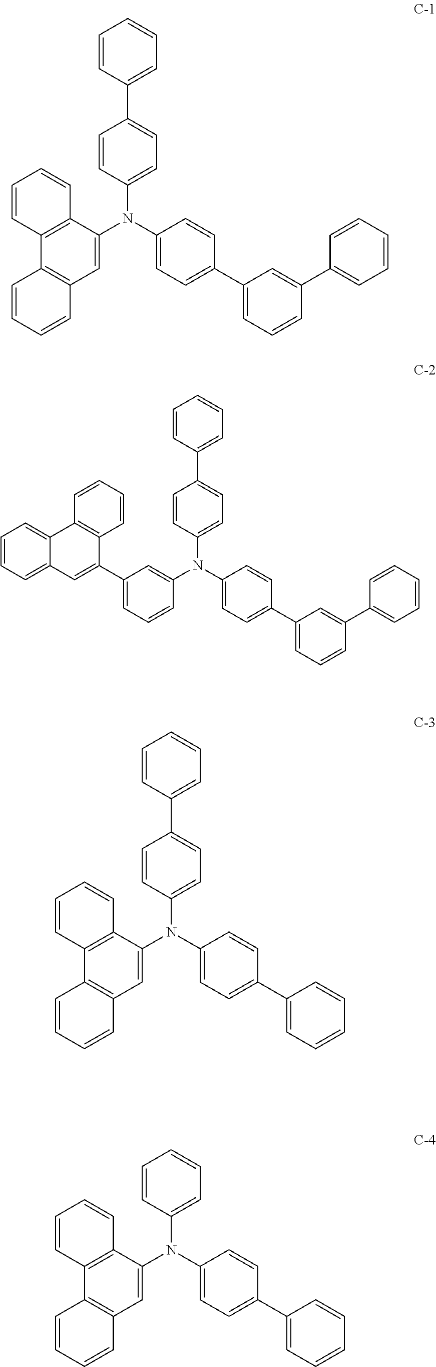 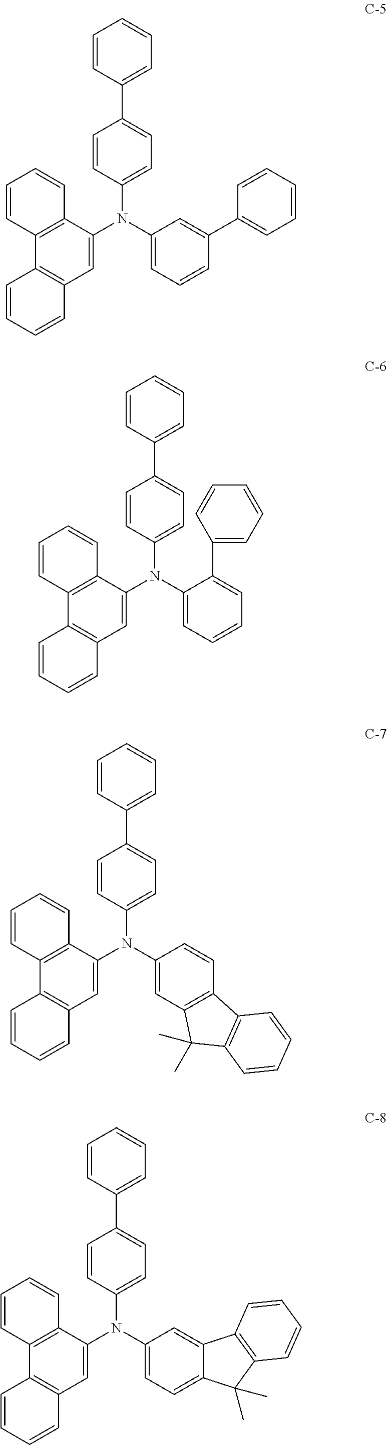 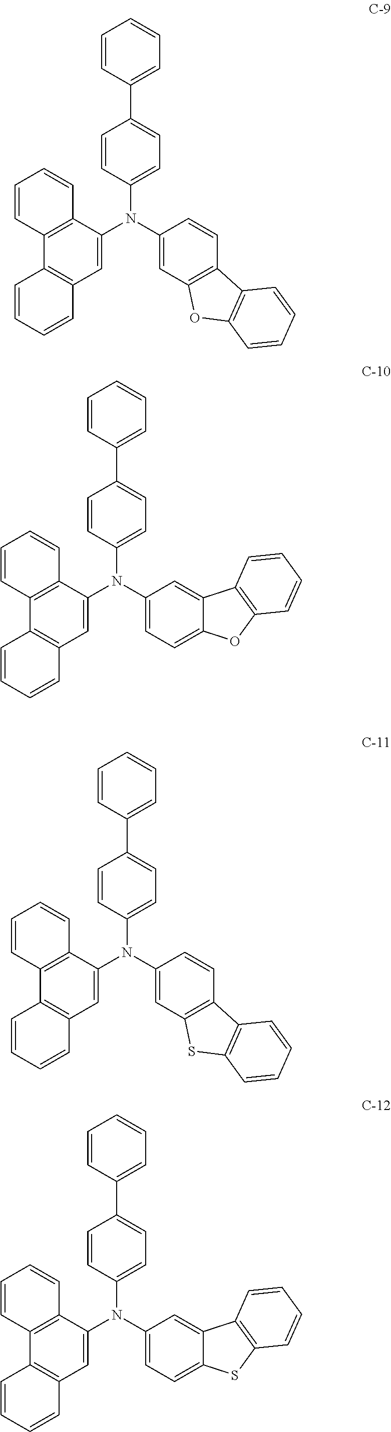 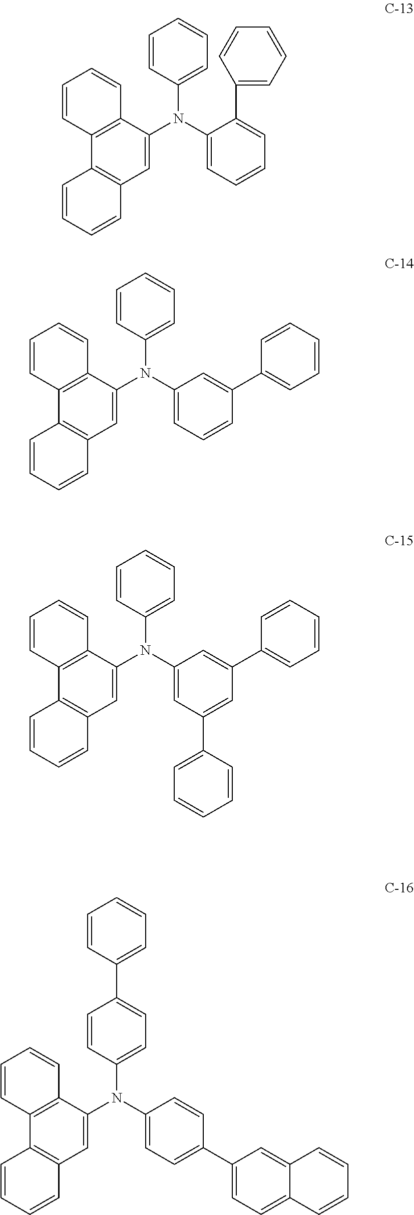 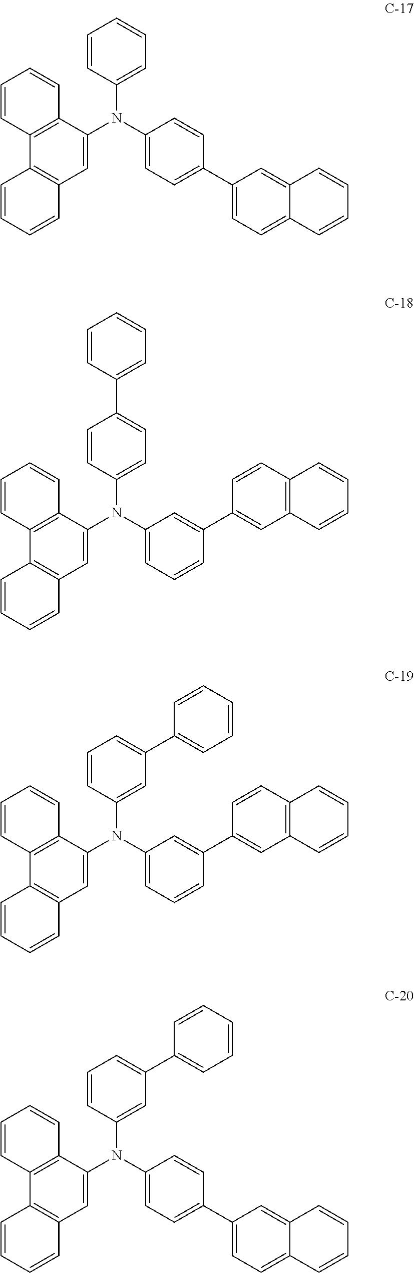 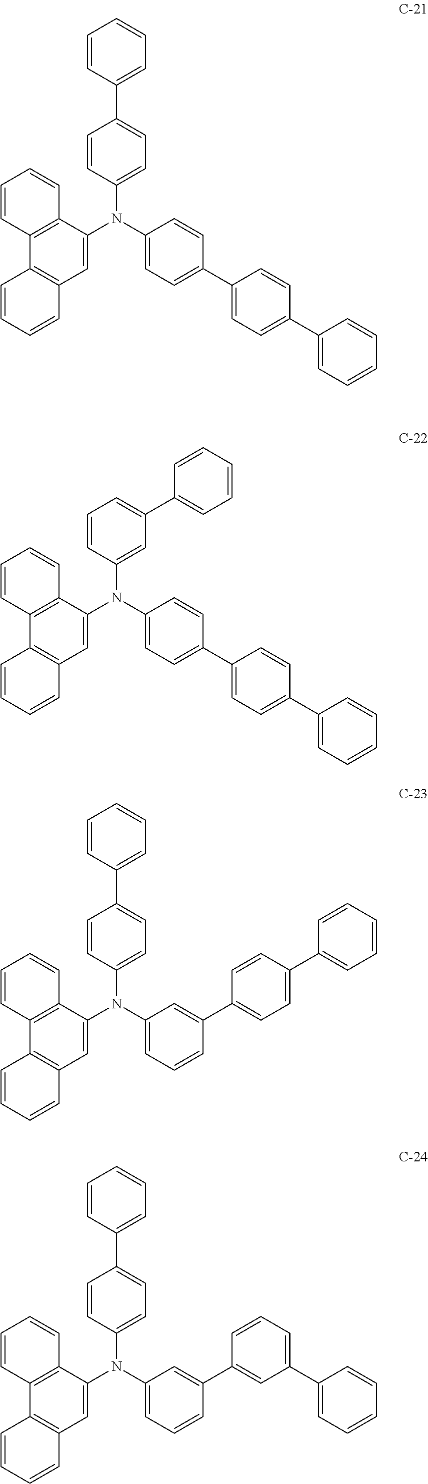 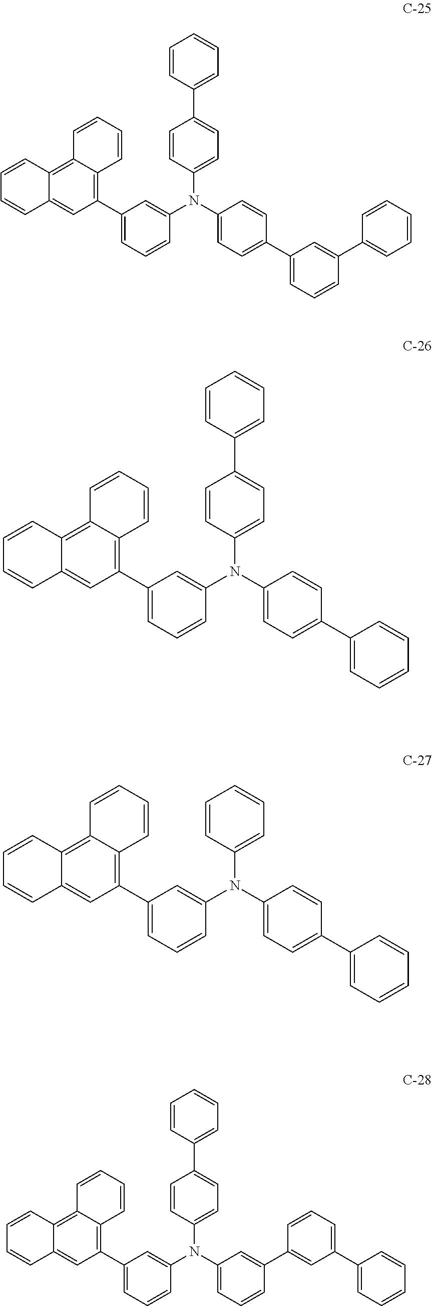 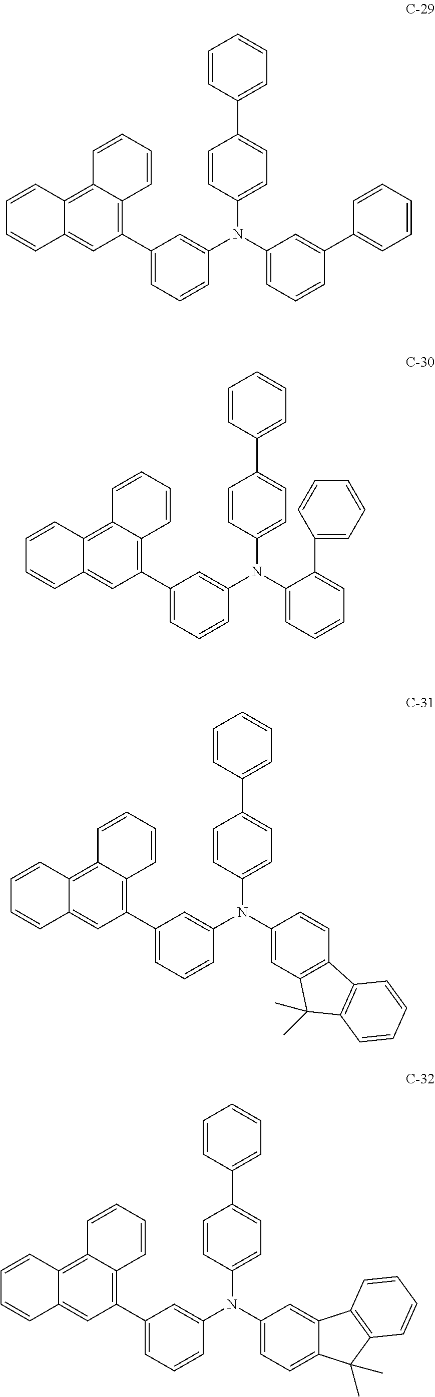 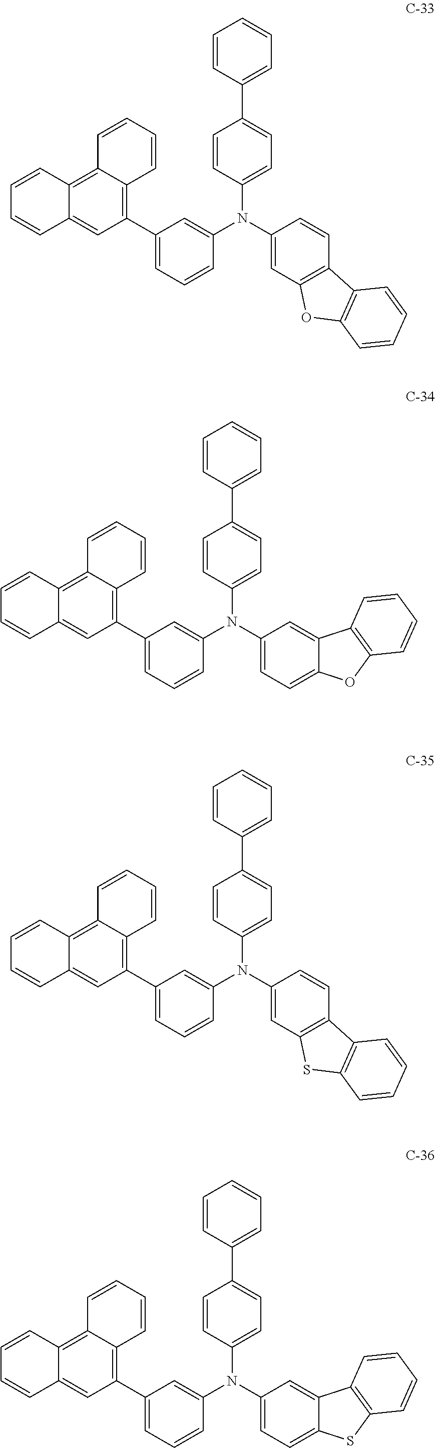 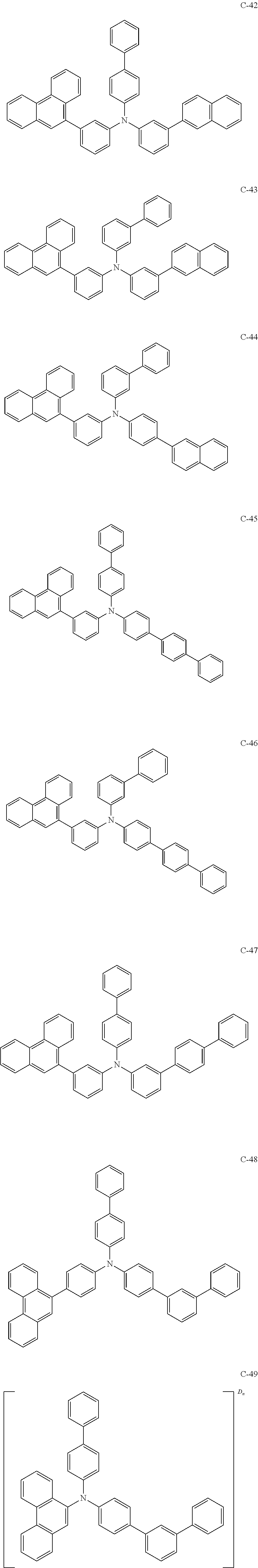 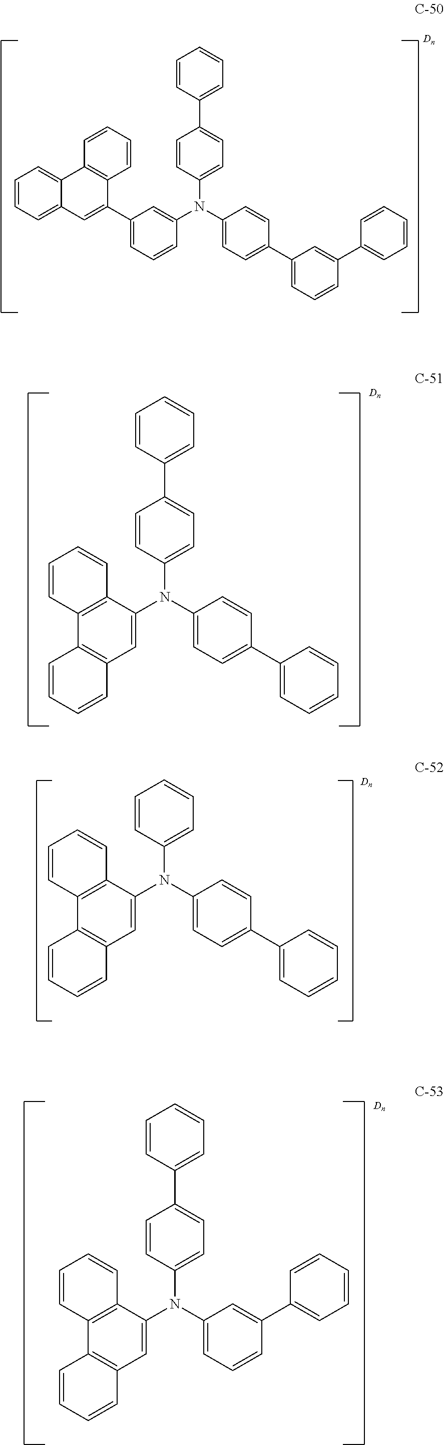 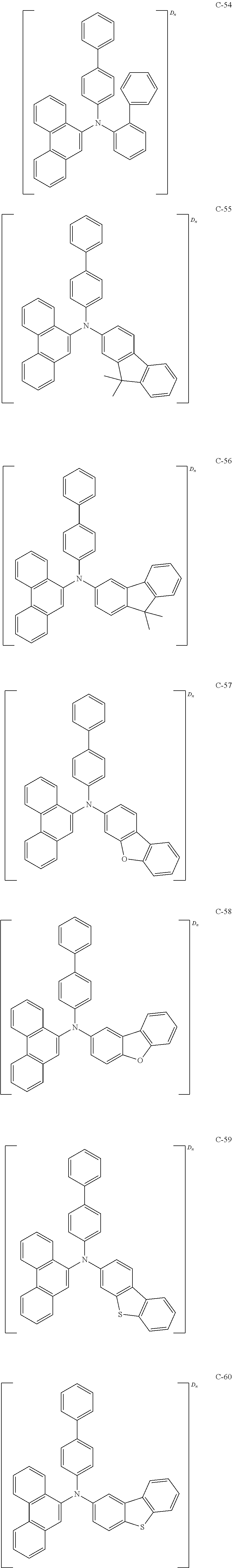 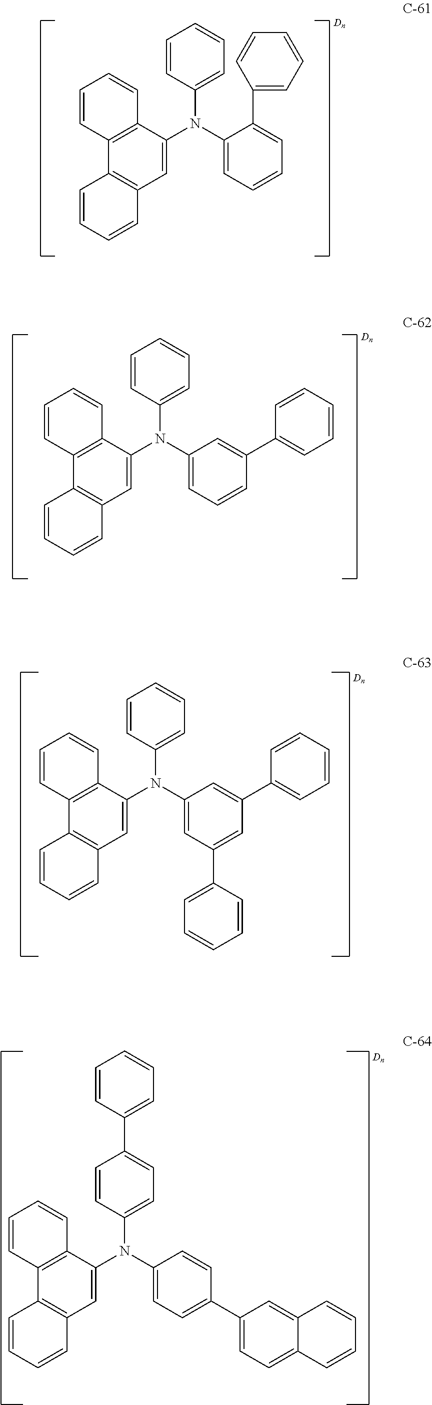 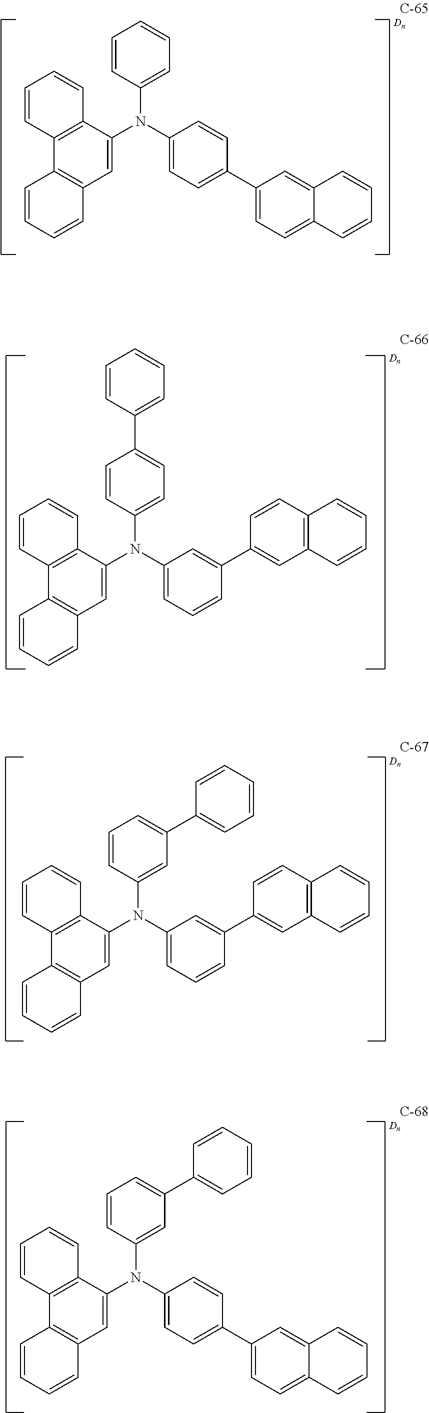 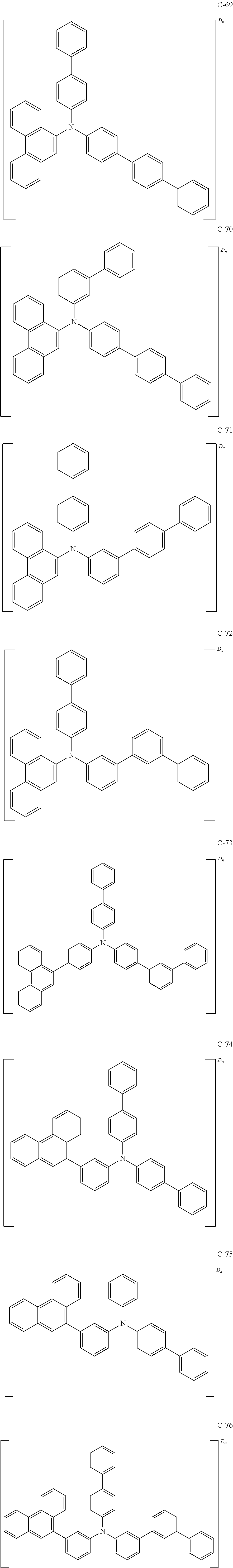 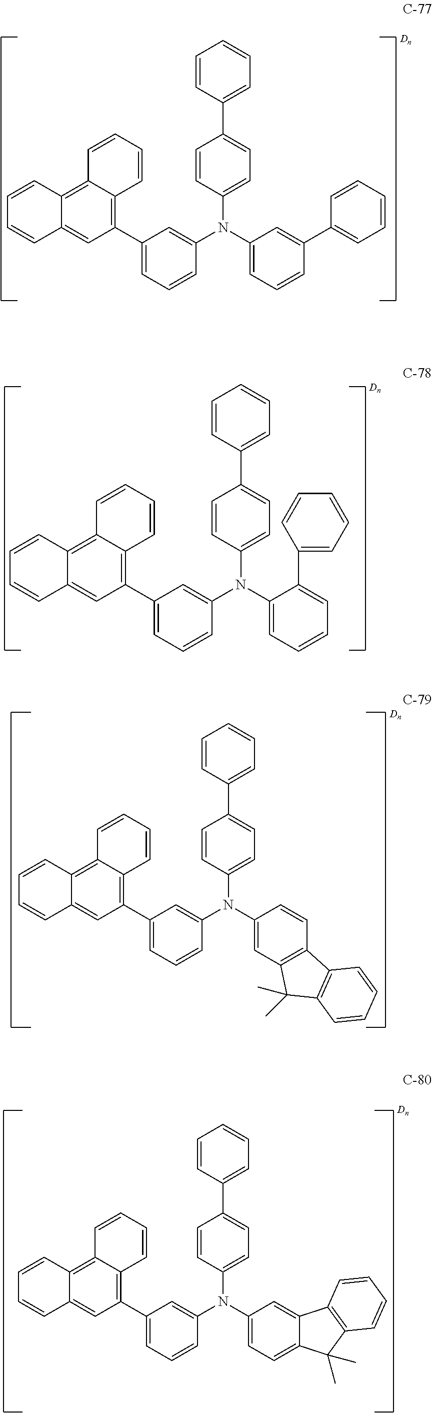 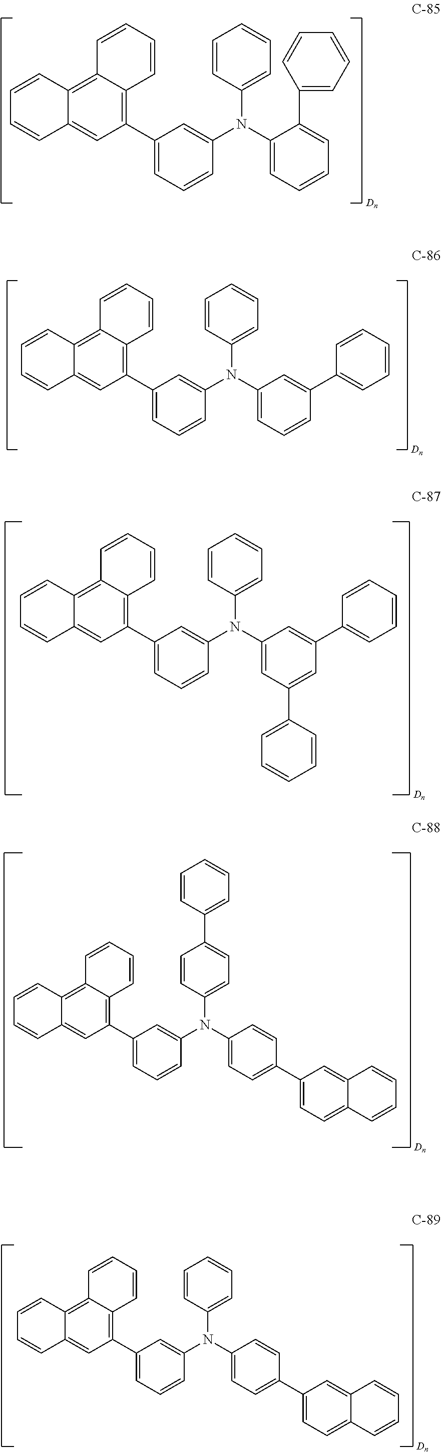 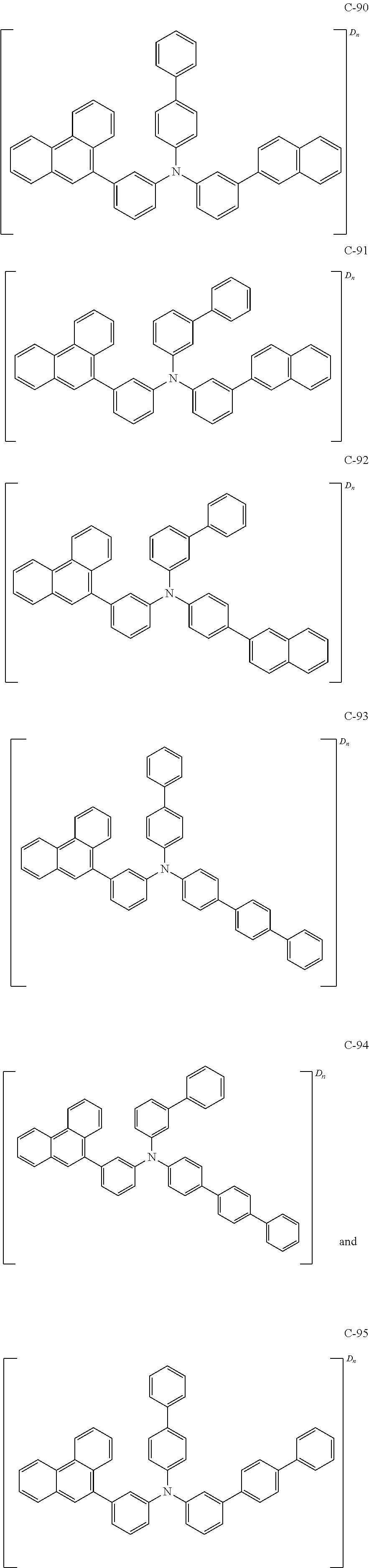
wherein, Dₙ represents that n hydrogens are replaced with deuterium; and n represents an integer of 1 or more.

8. The plurality of organic electroluminescent materials according to claim 1, wherein the compound represented by formula 2 is at least one selected from the following compounds: 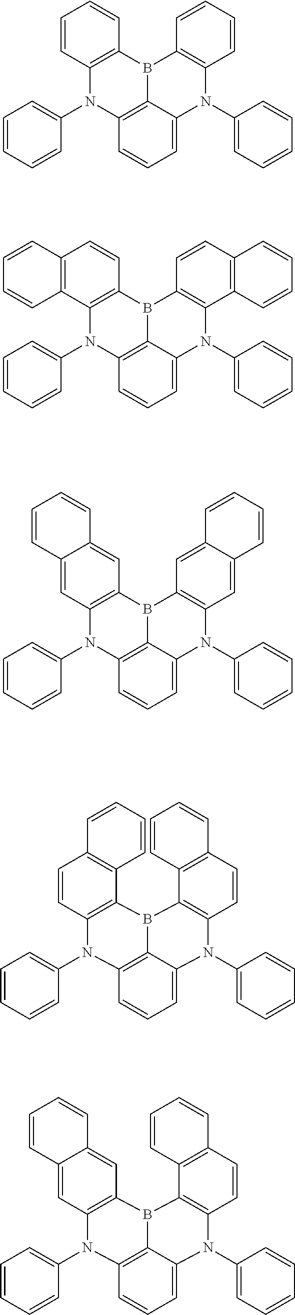 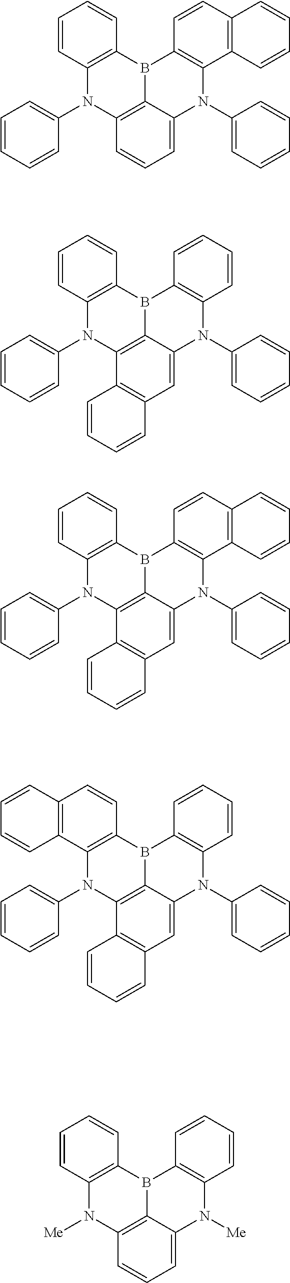 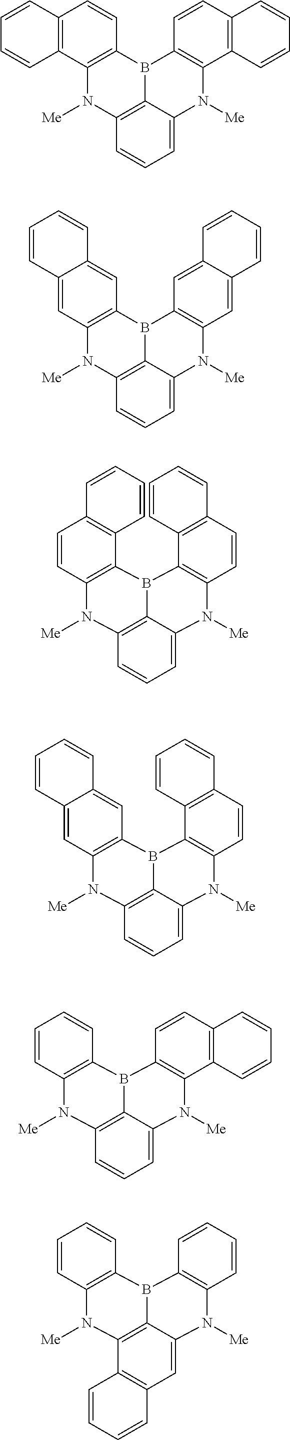 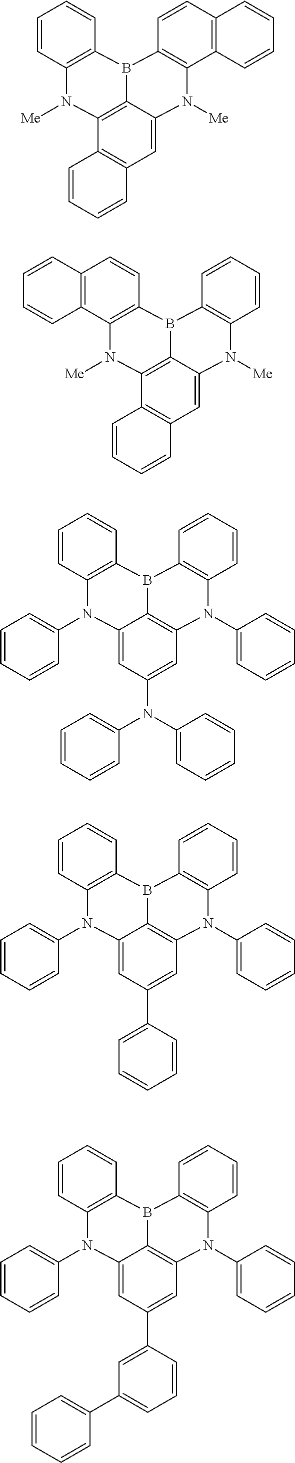 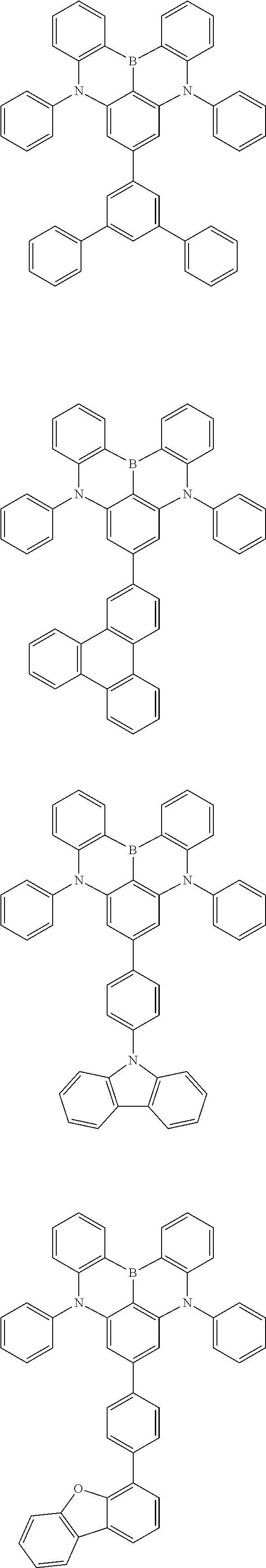 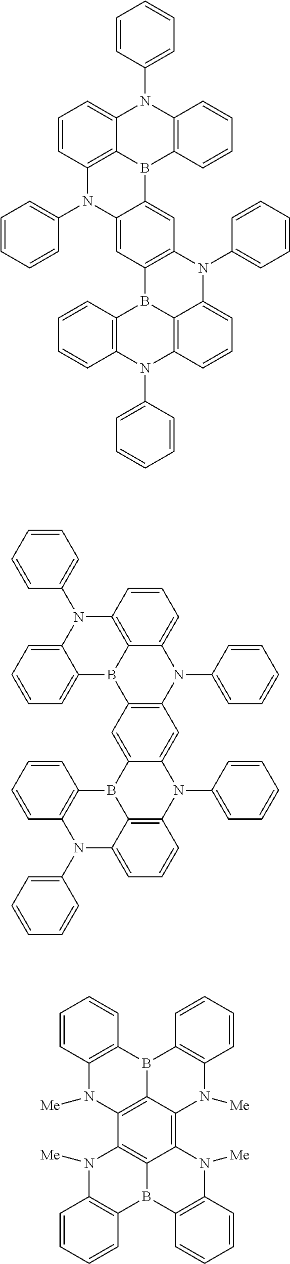 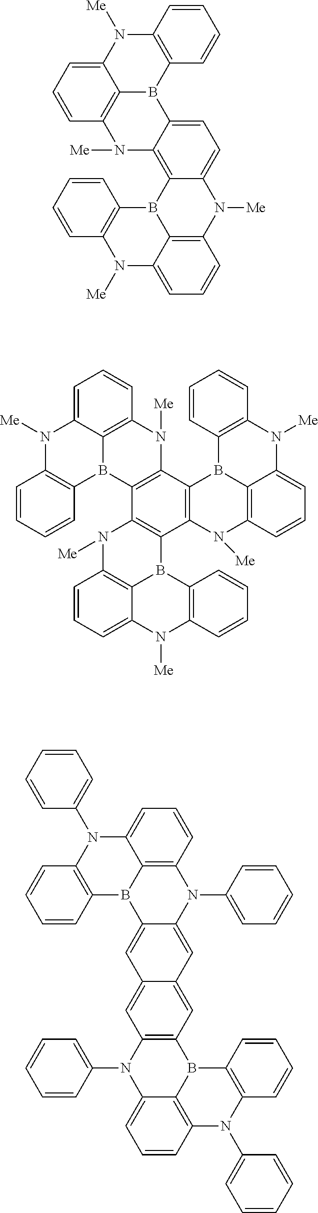 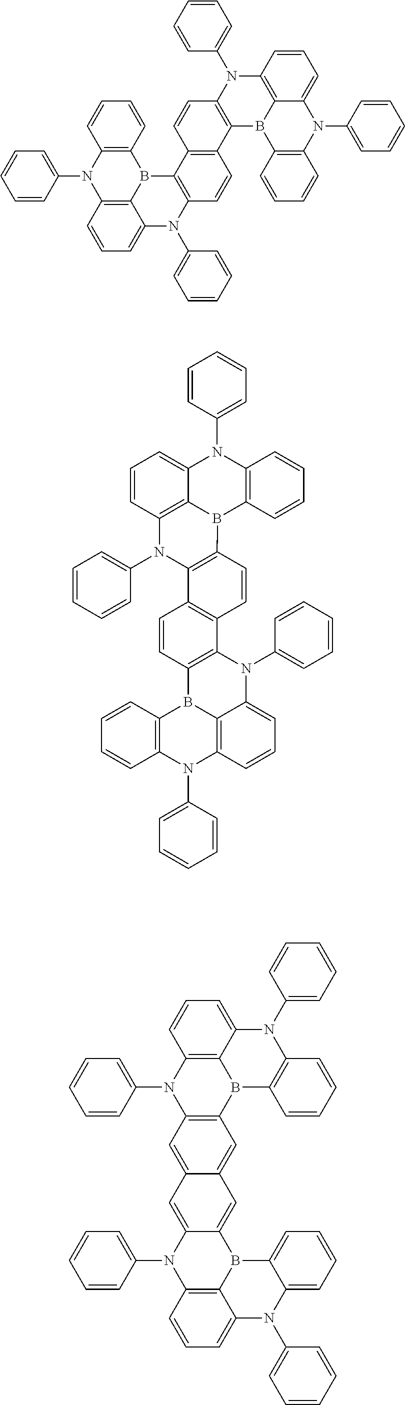 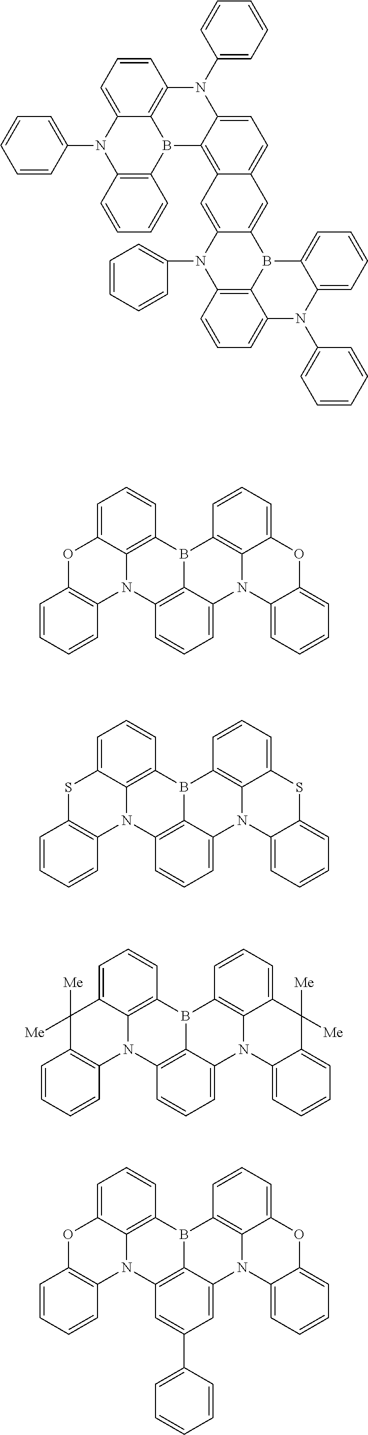 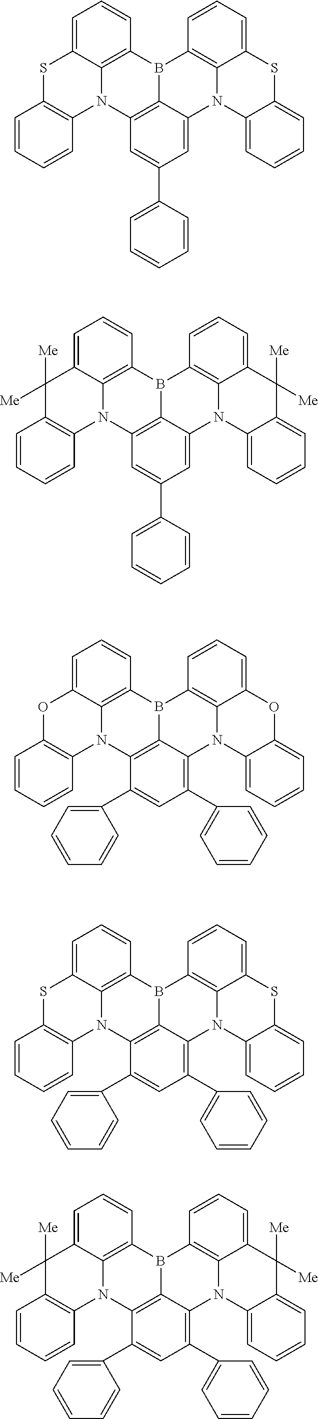 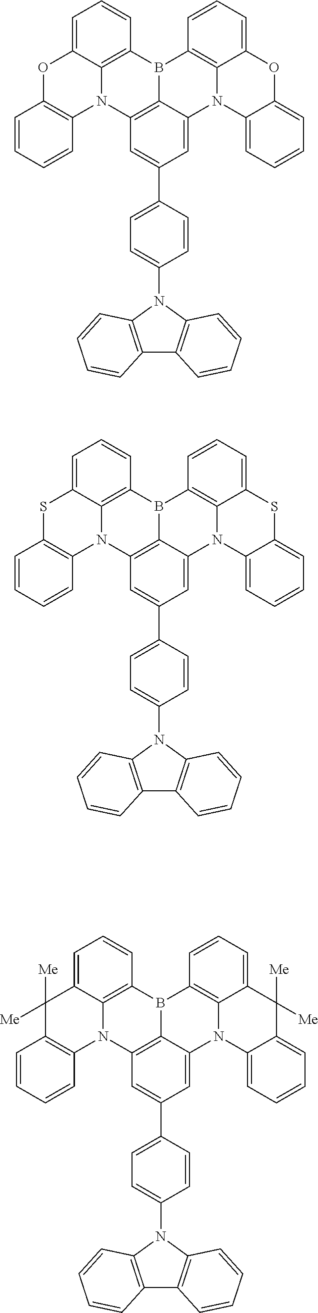 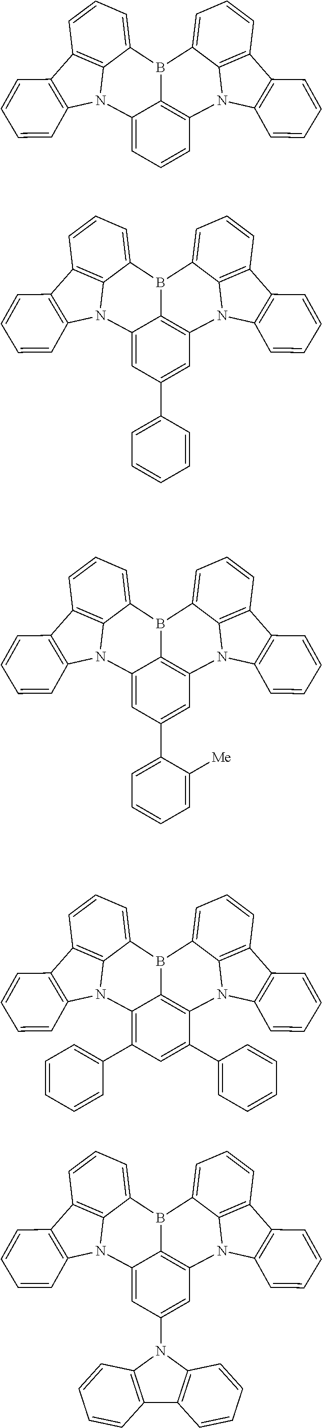 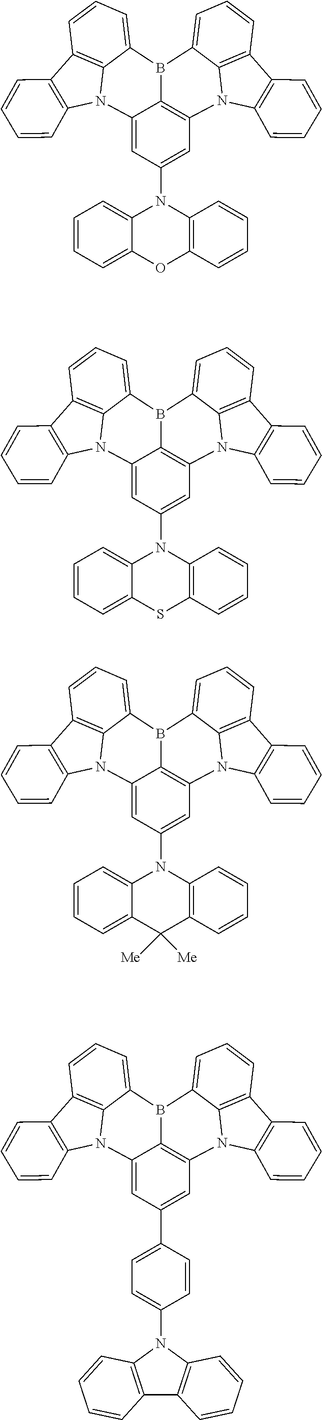 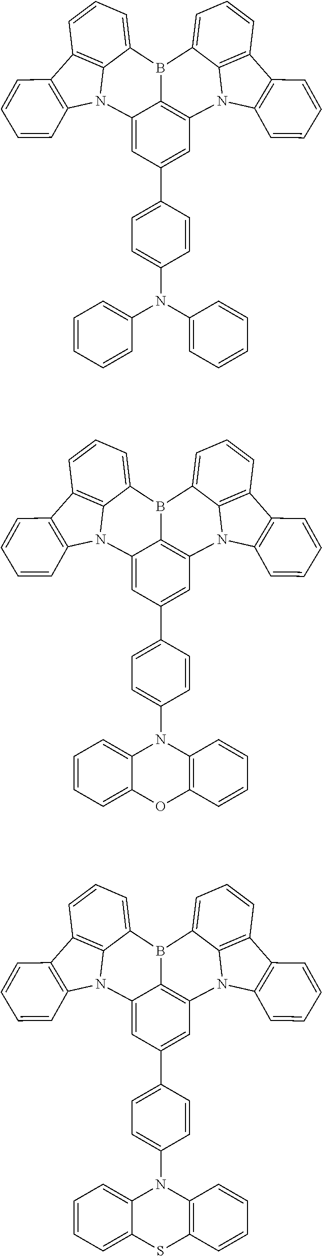 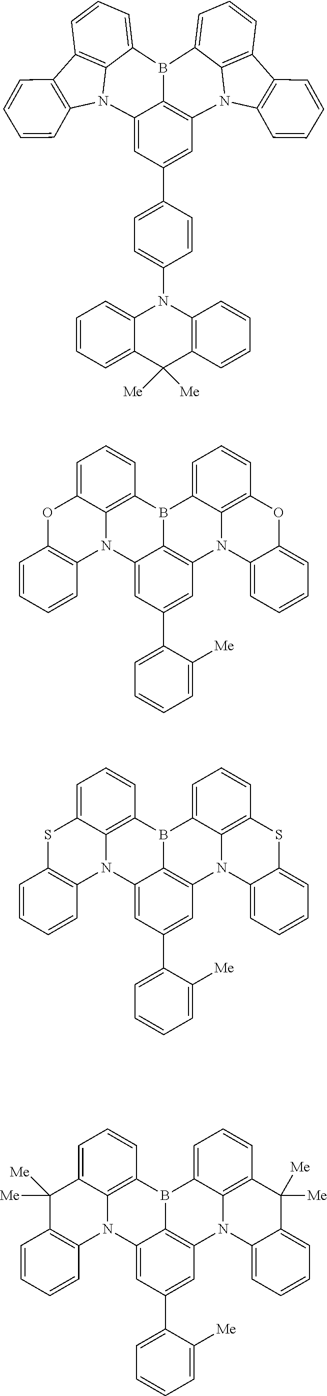 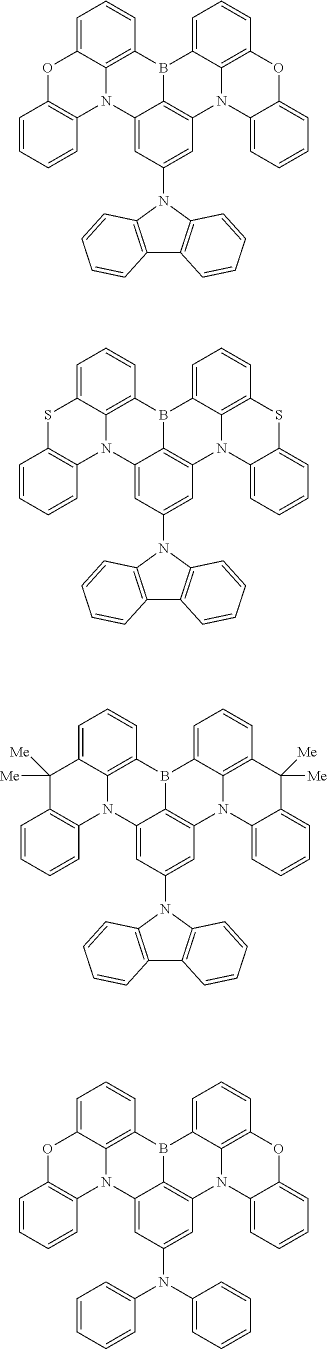 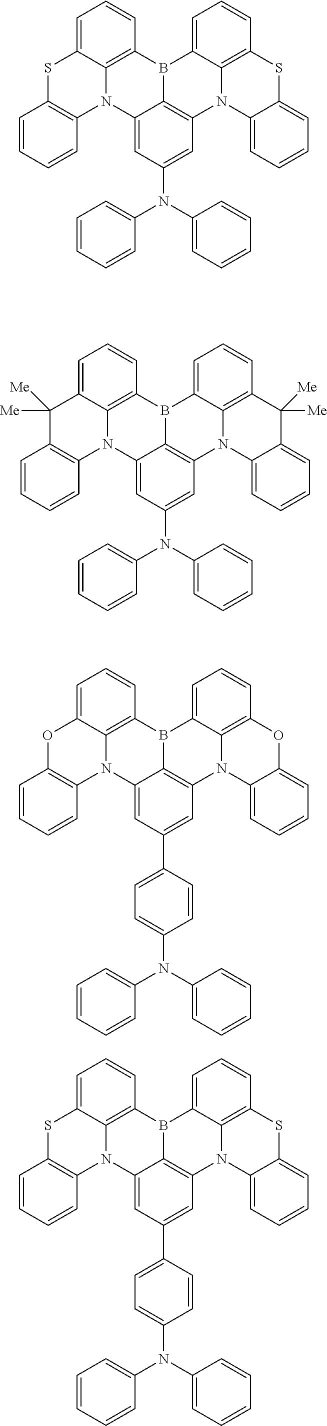 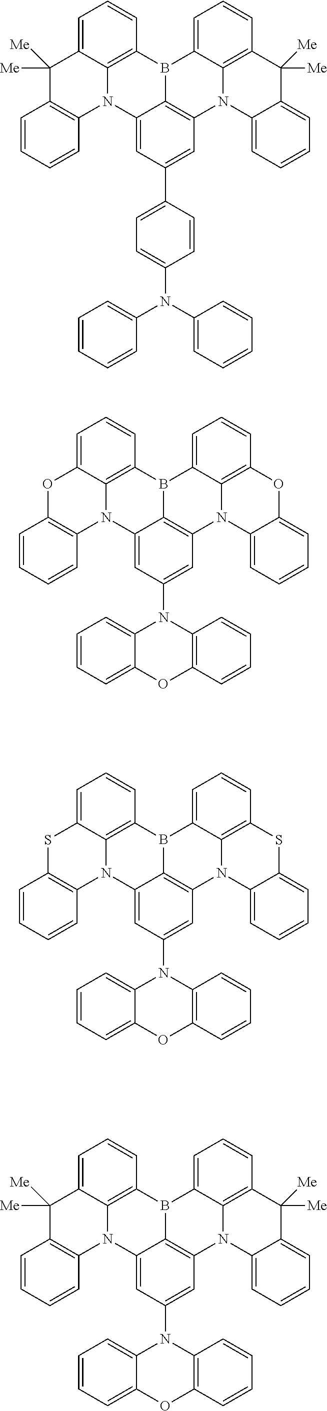 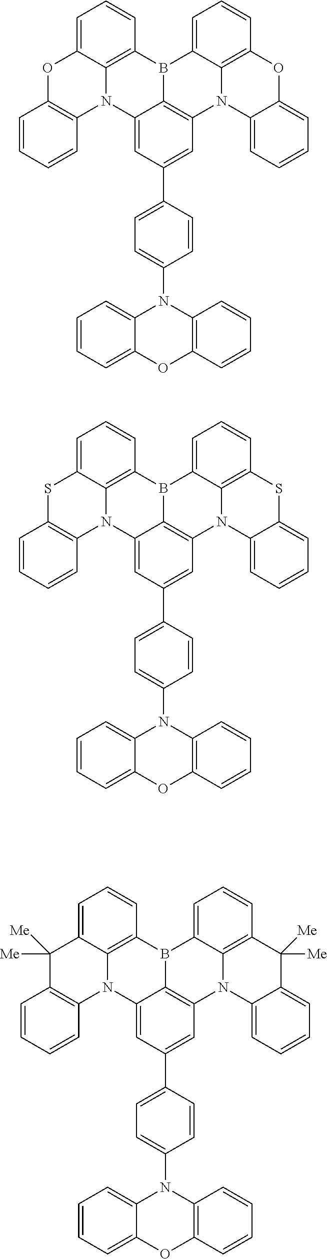 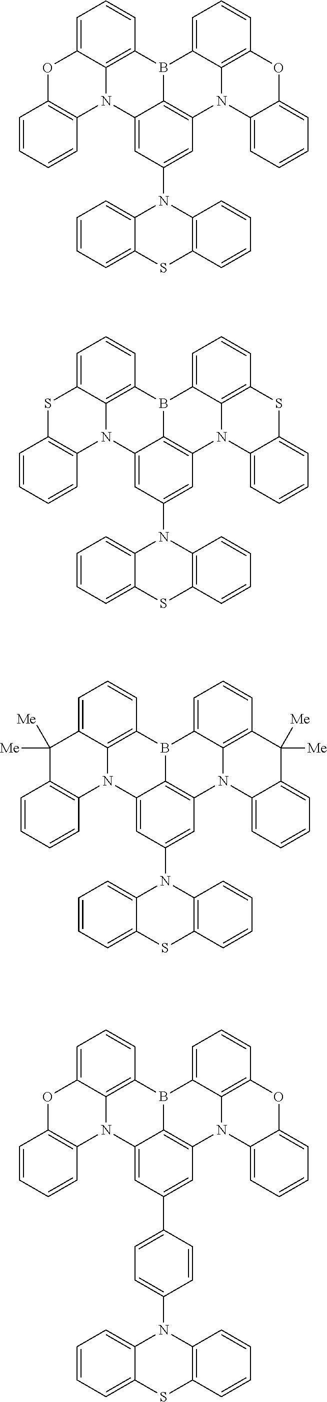 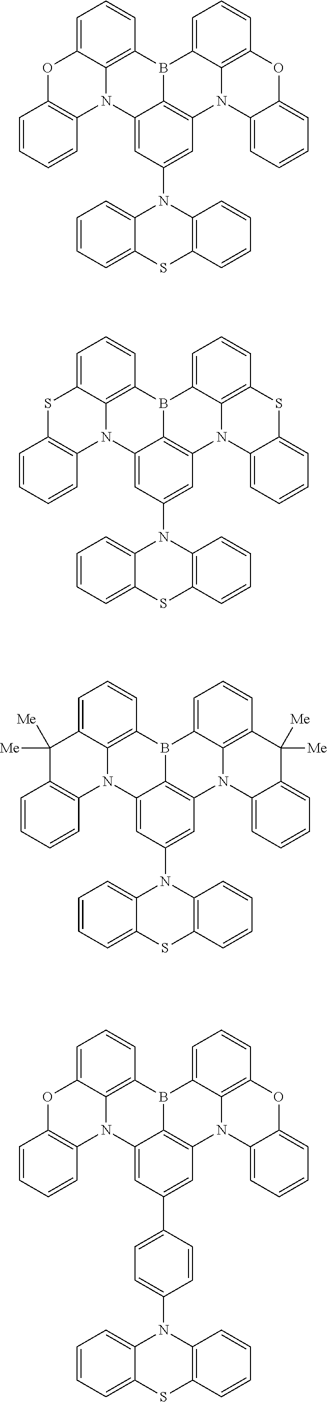 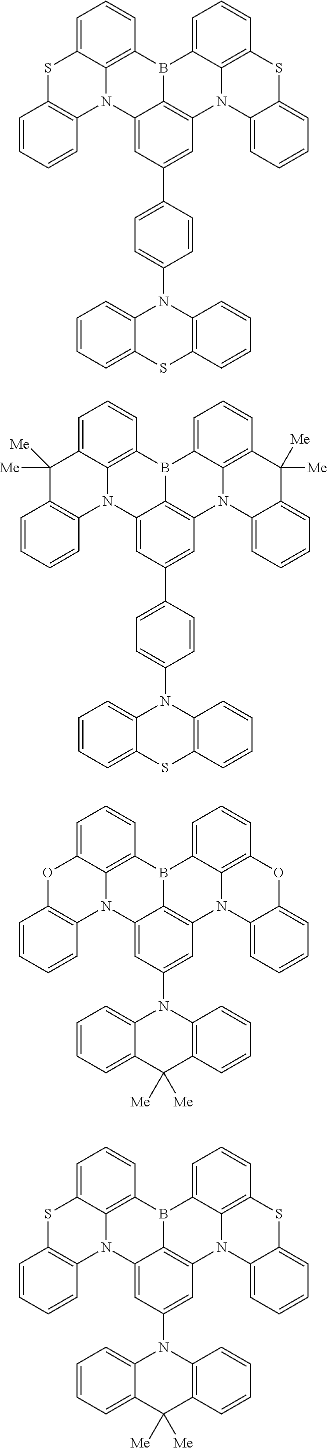 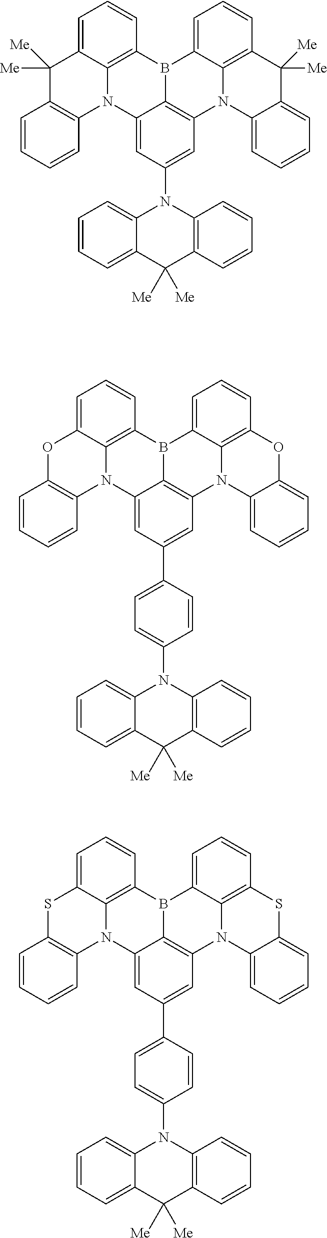 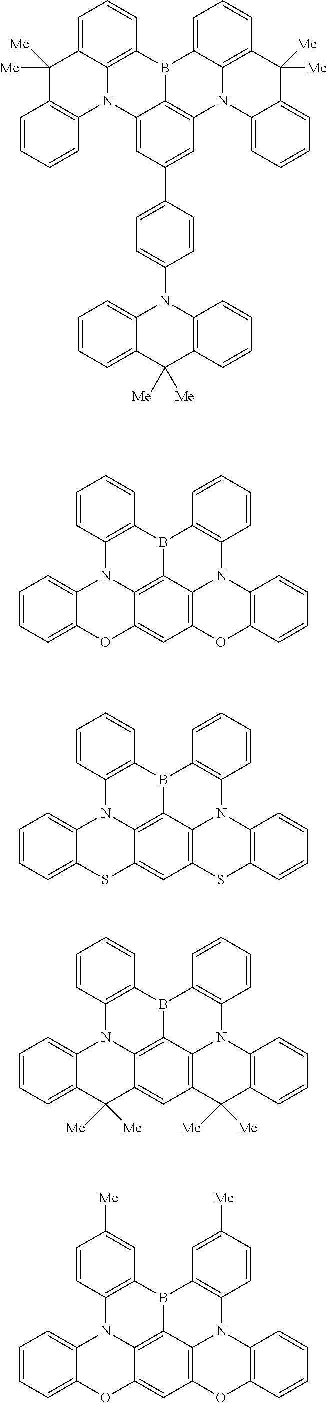 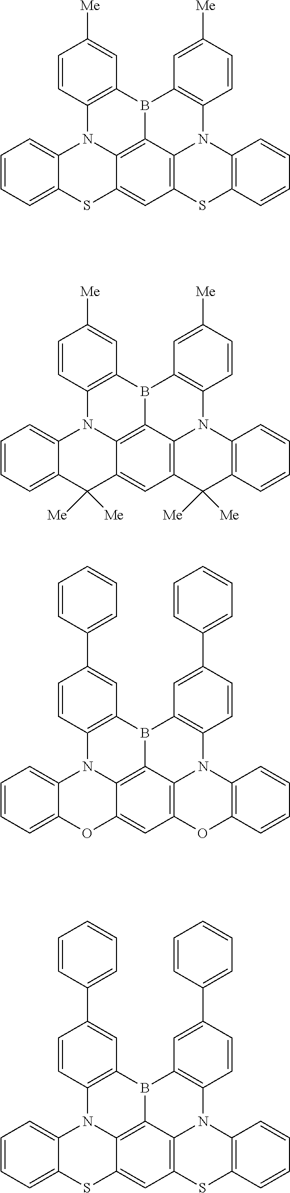 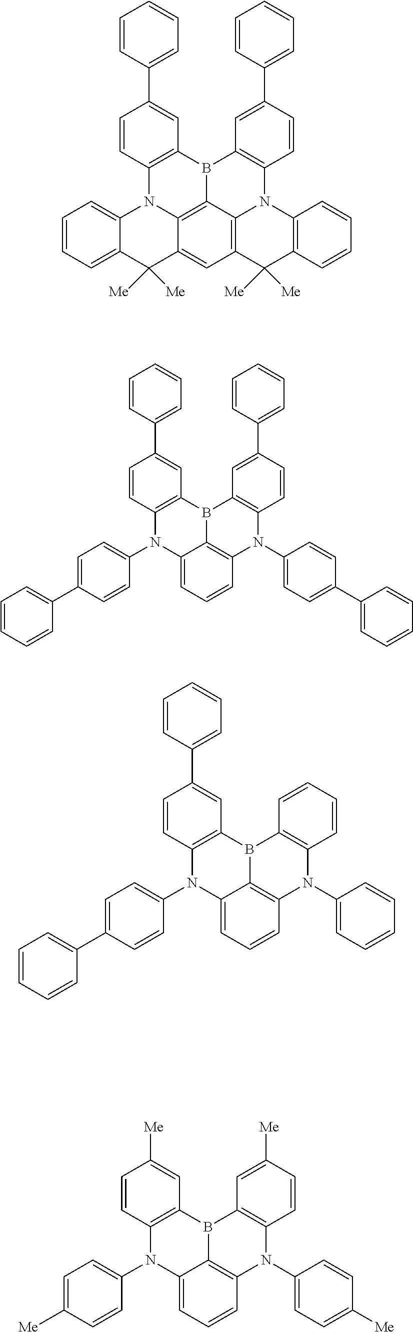 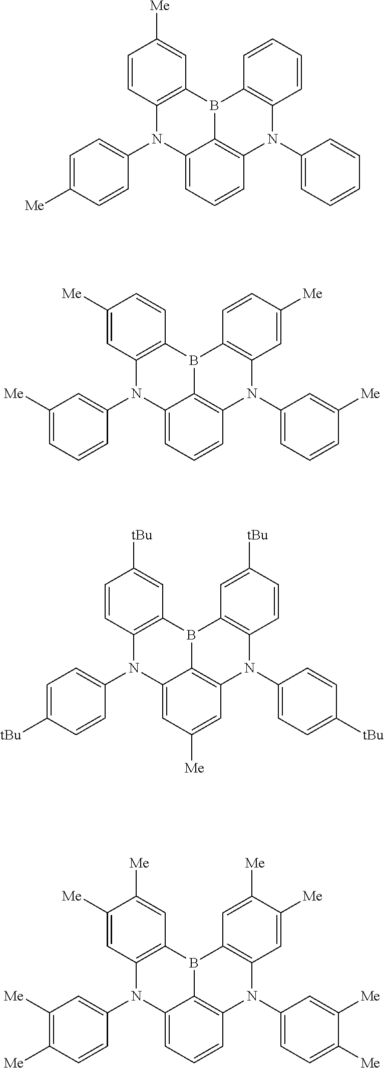 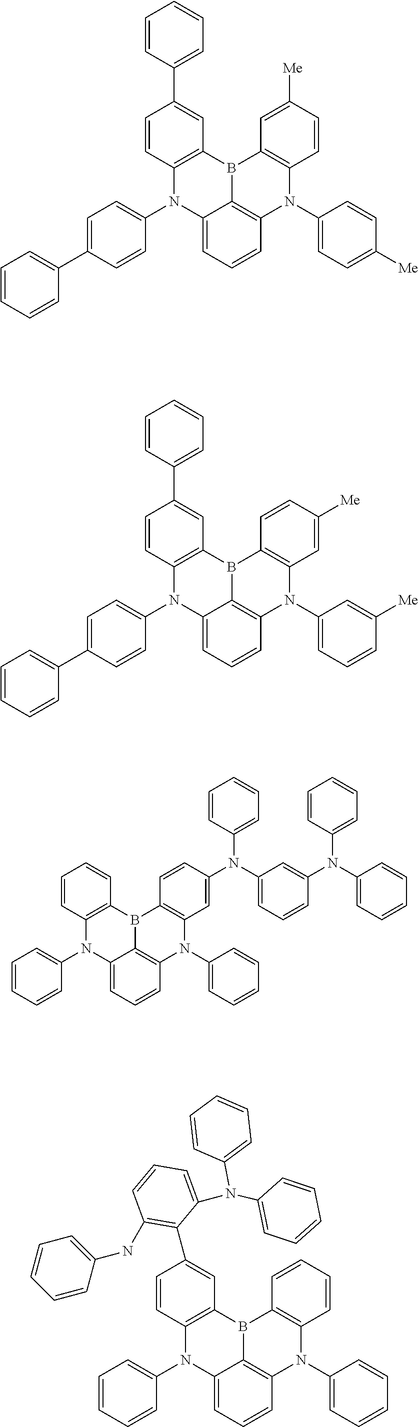 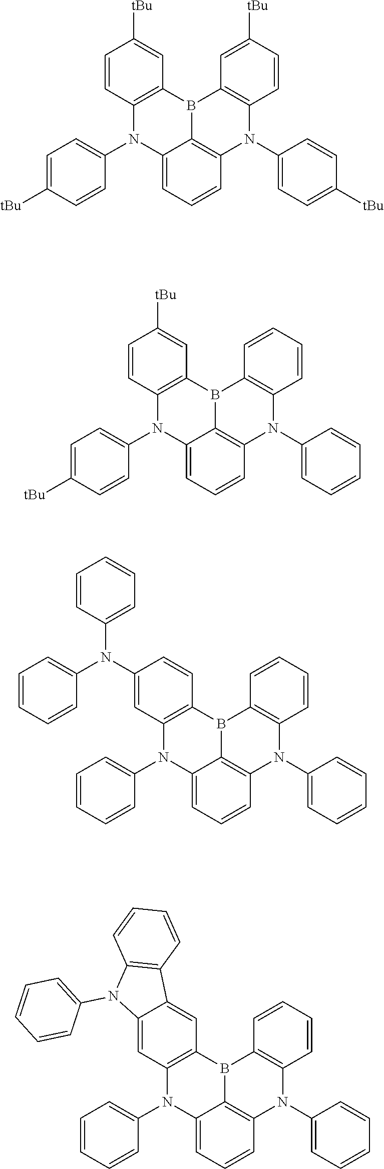 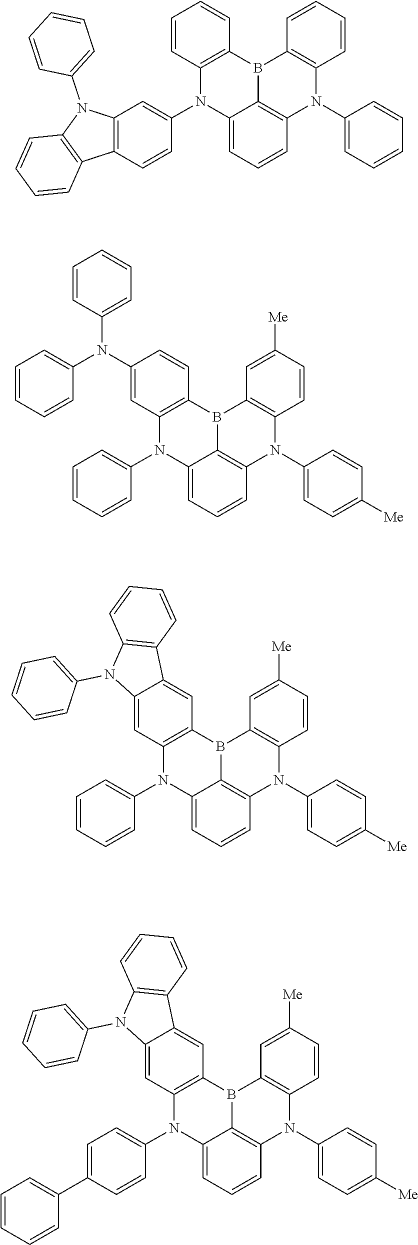 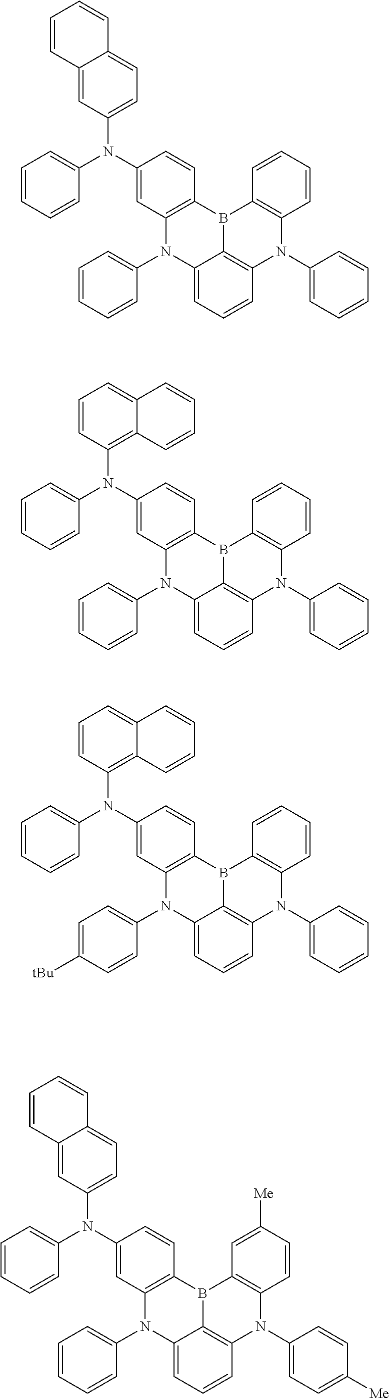 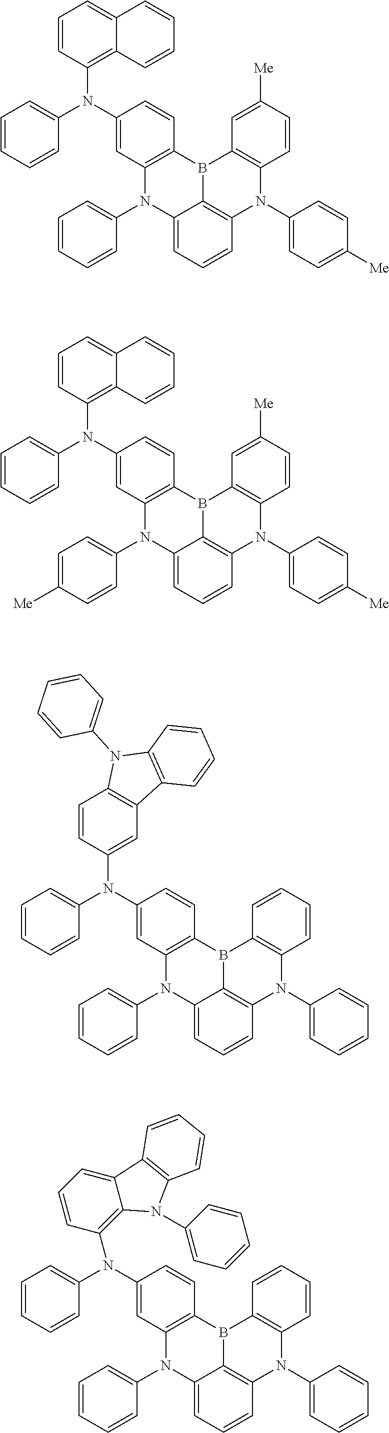 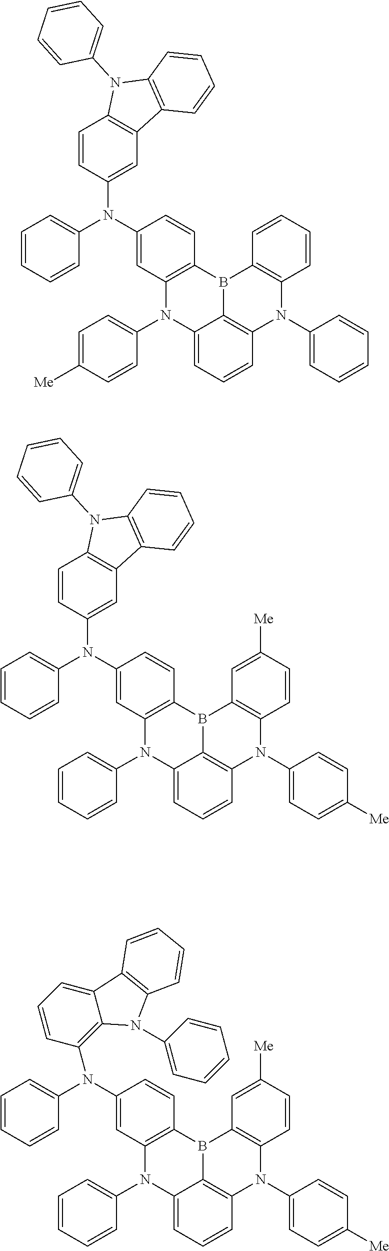 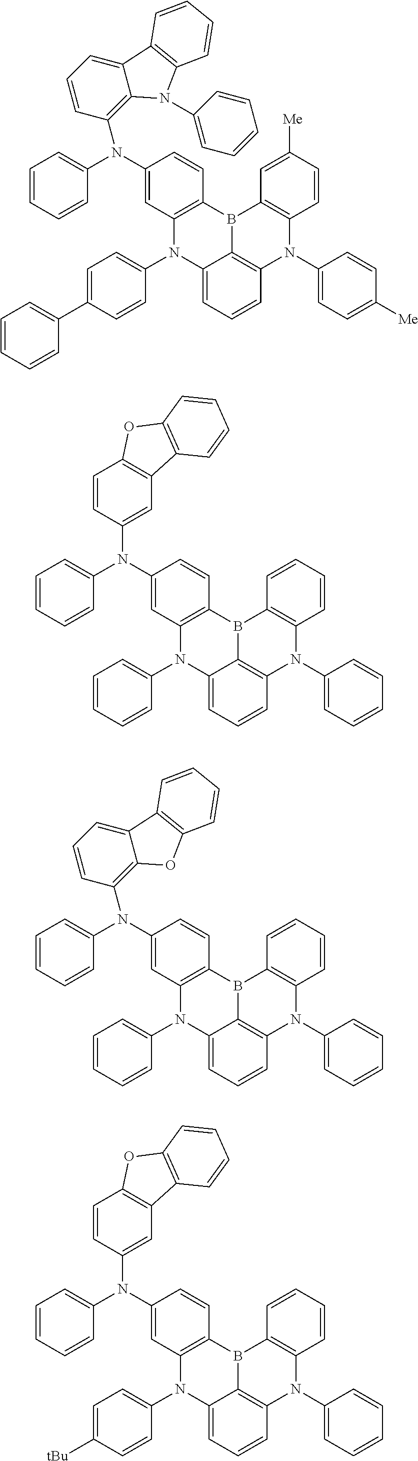 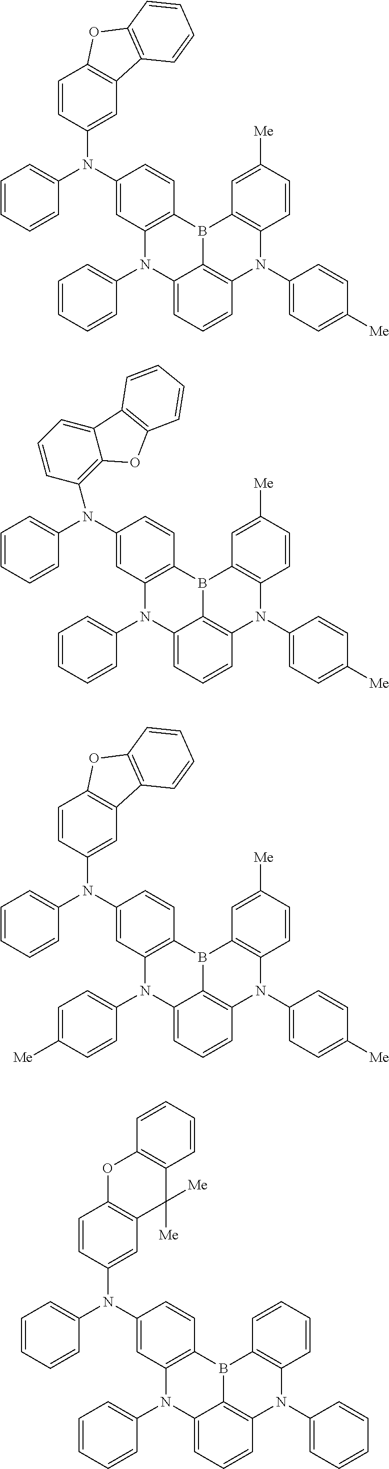 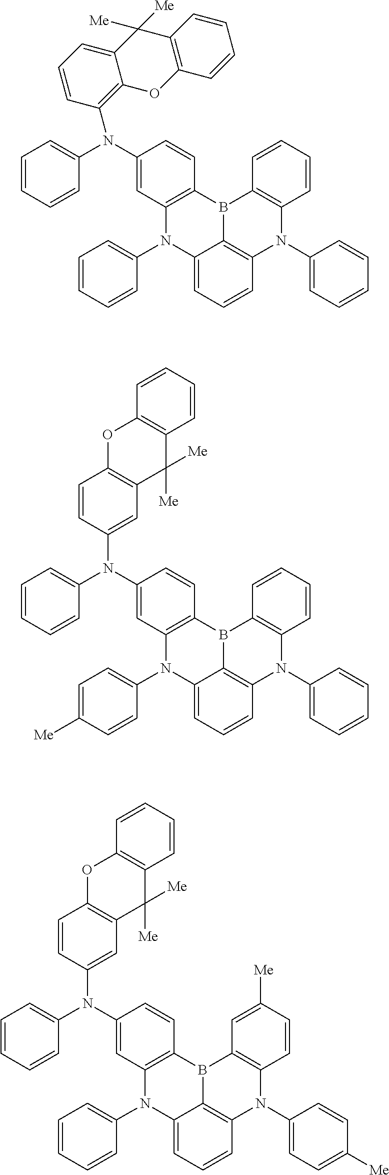 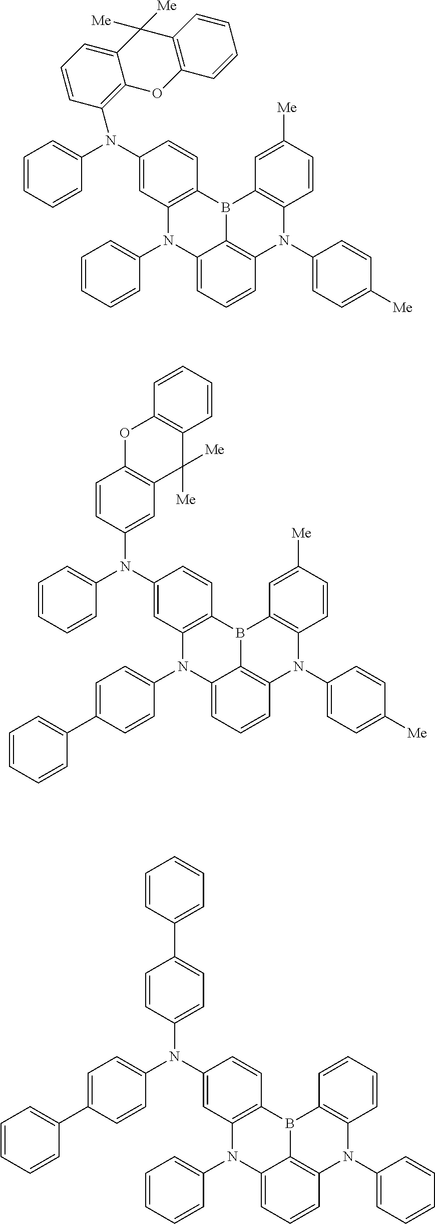 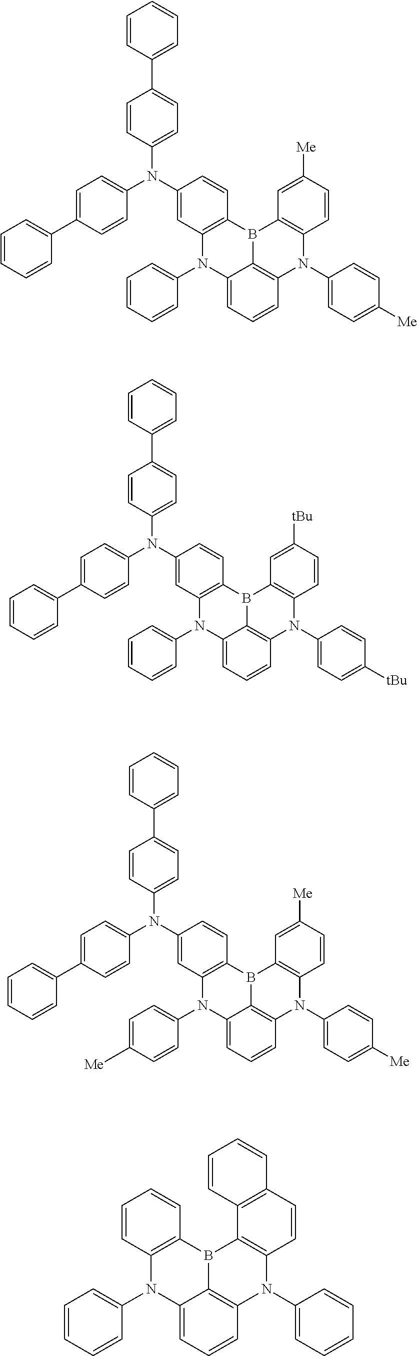 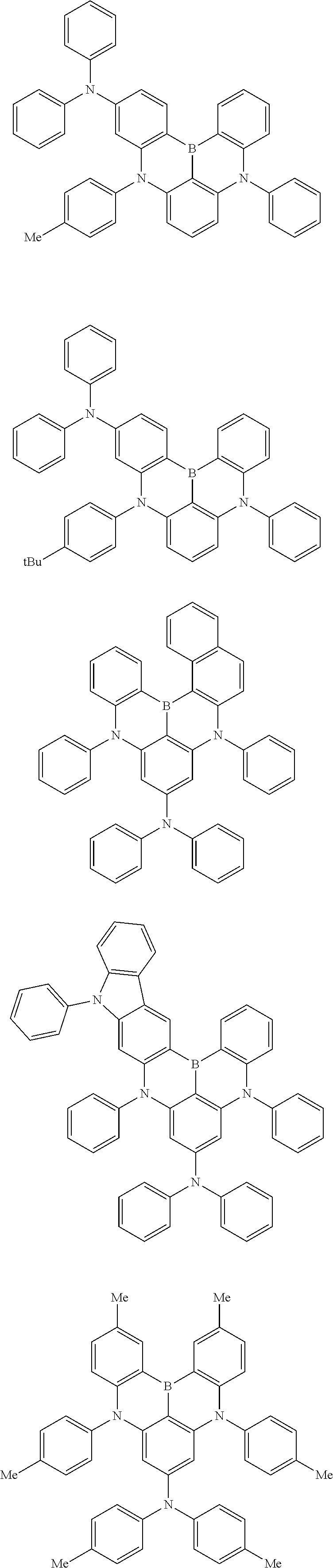 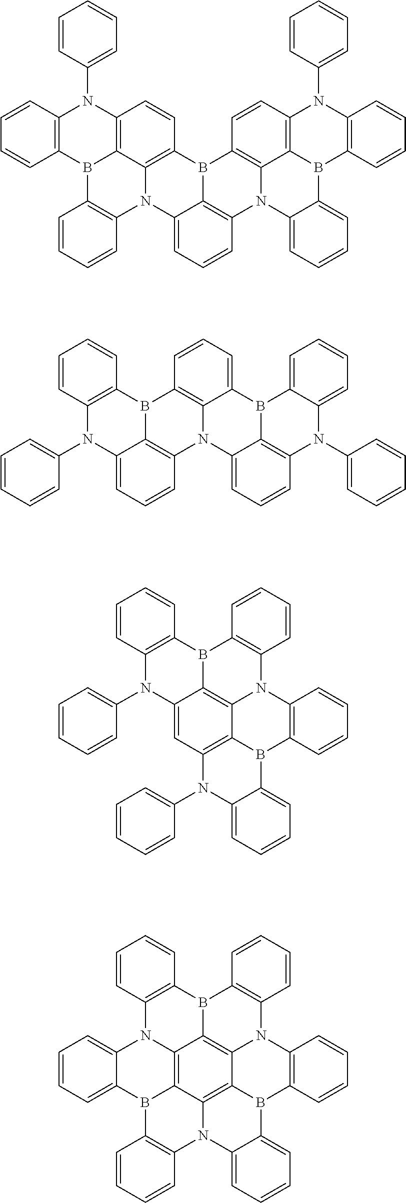 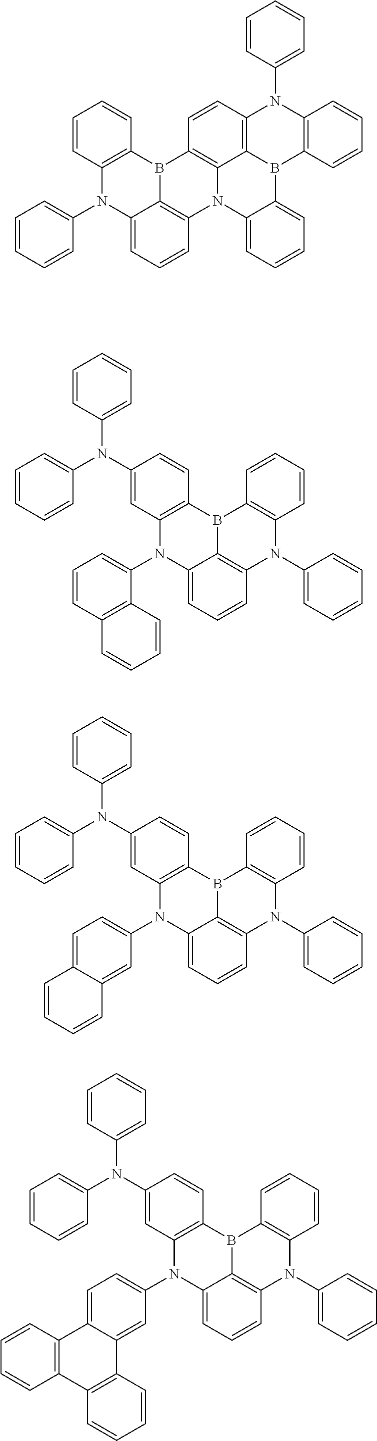 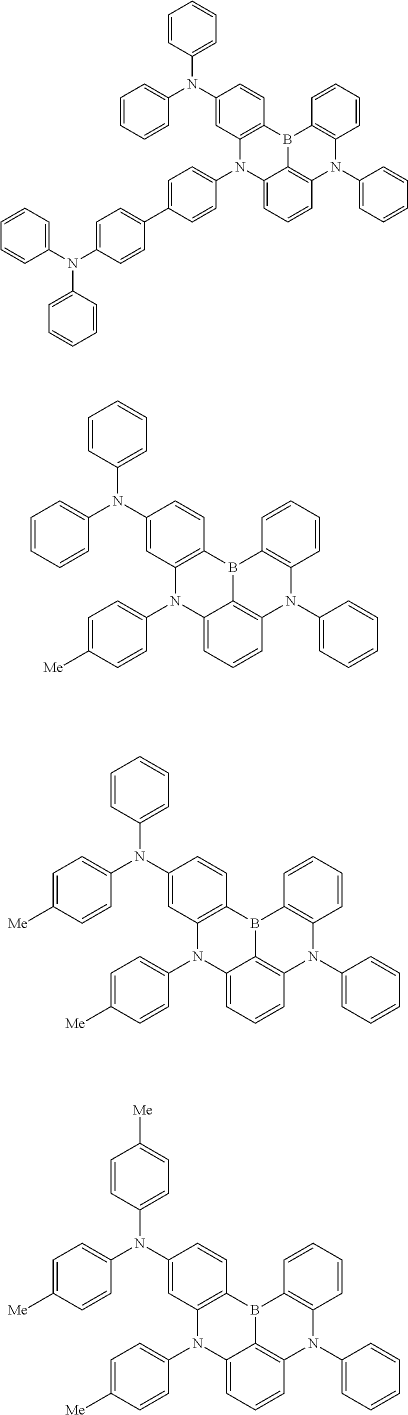 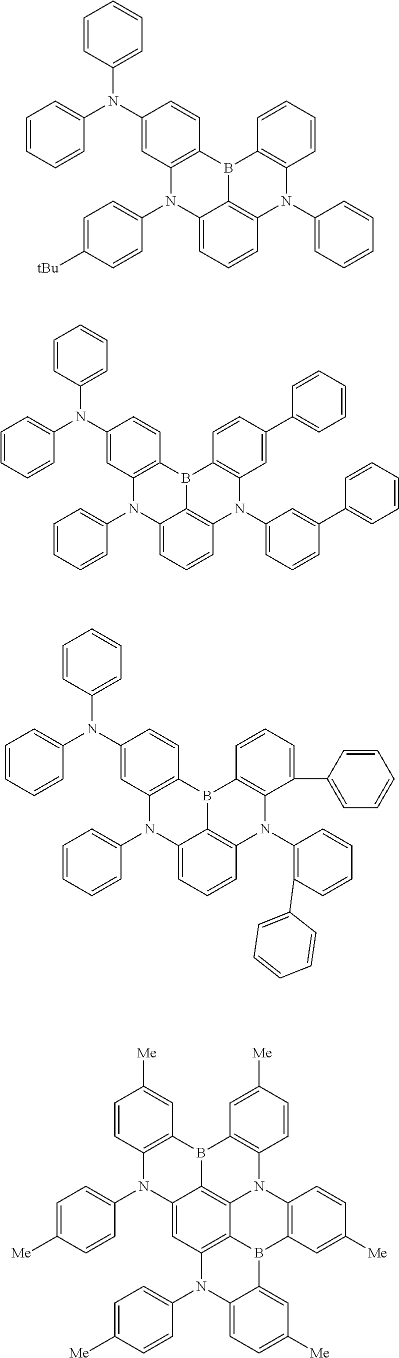 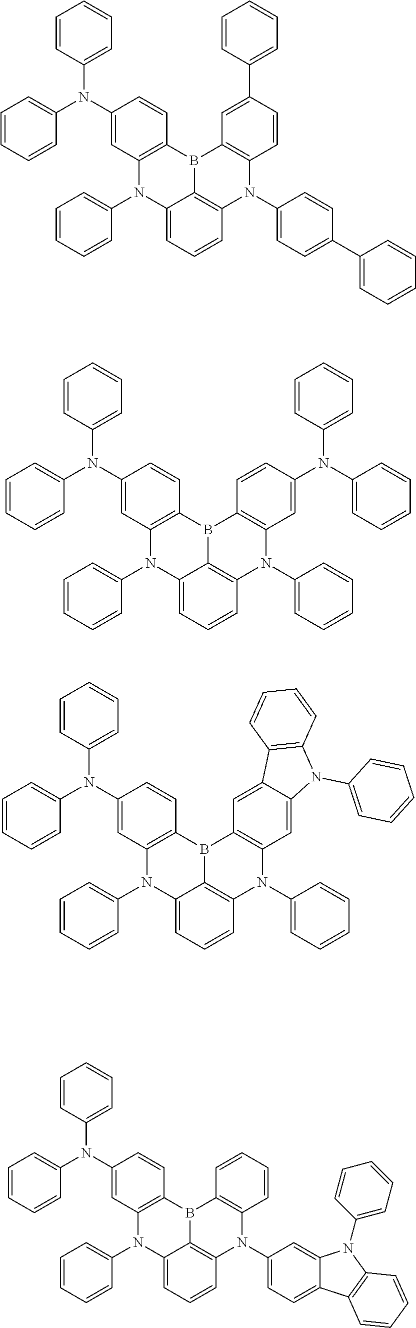 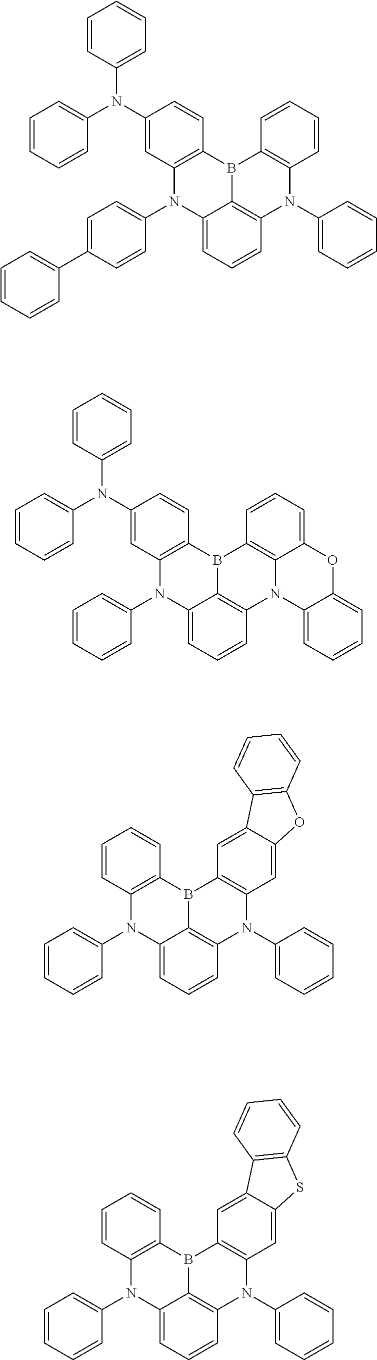 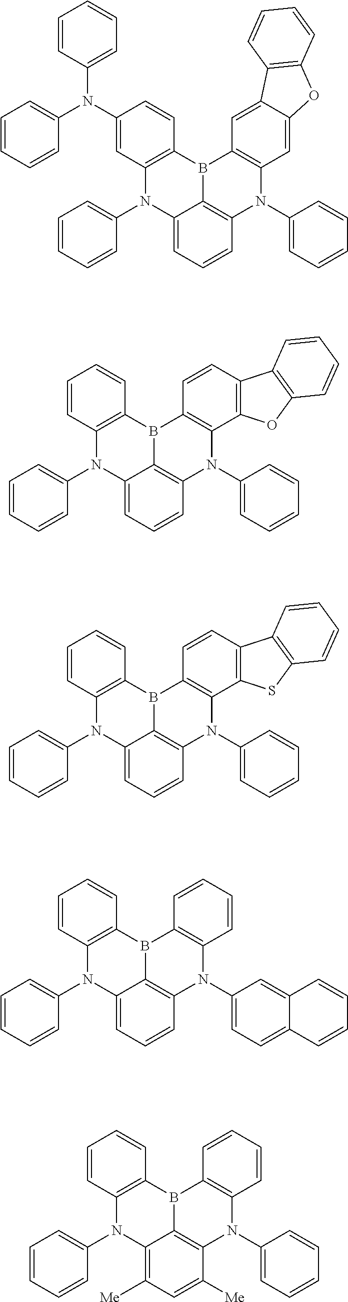 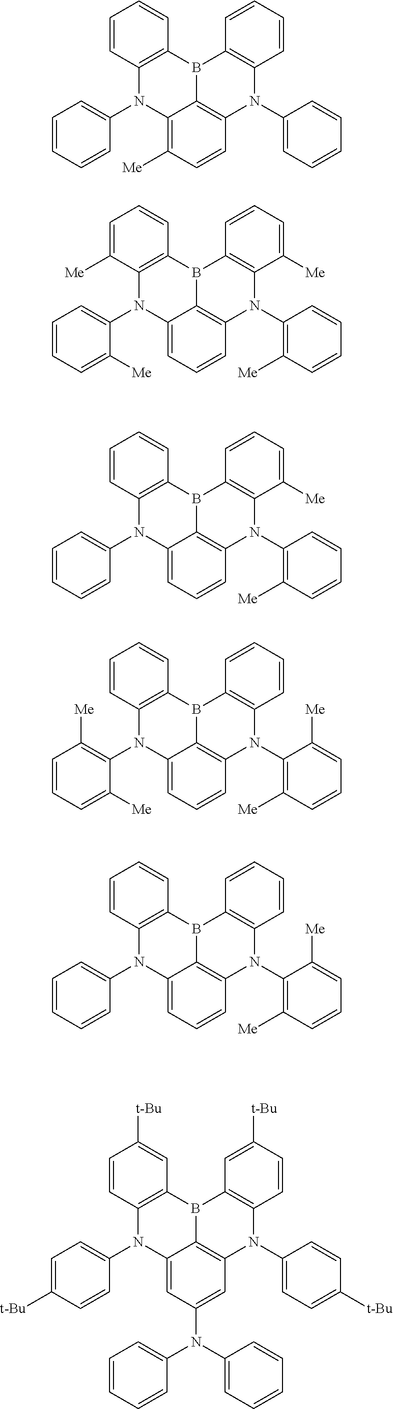 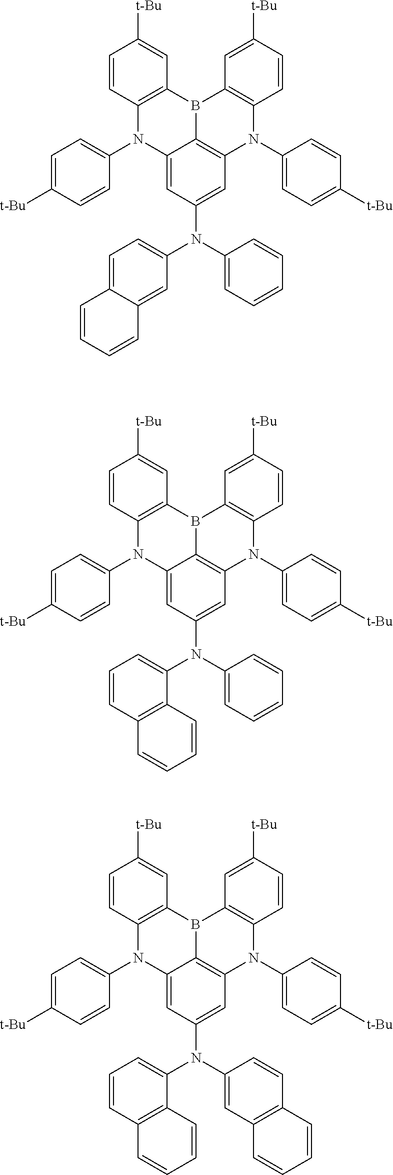 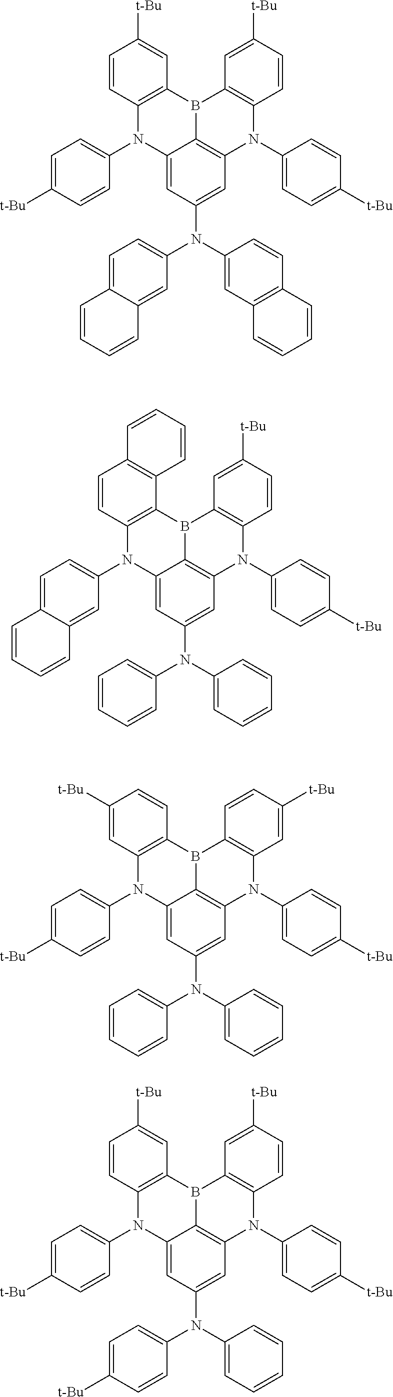 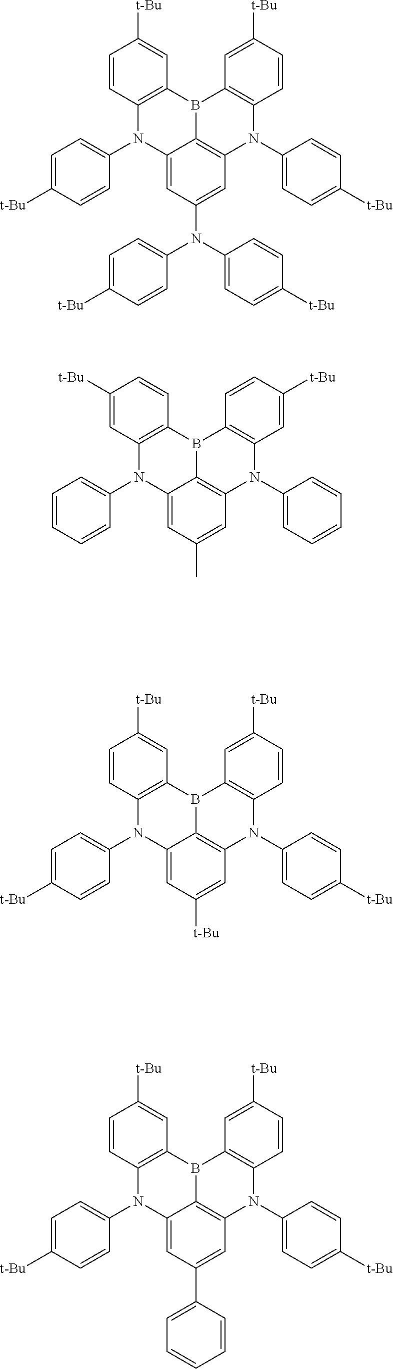 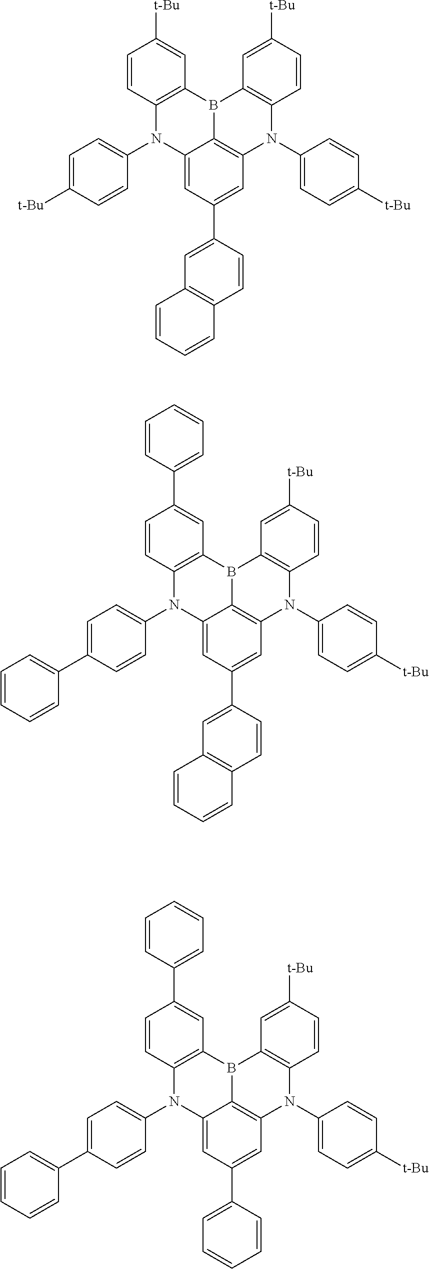 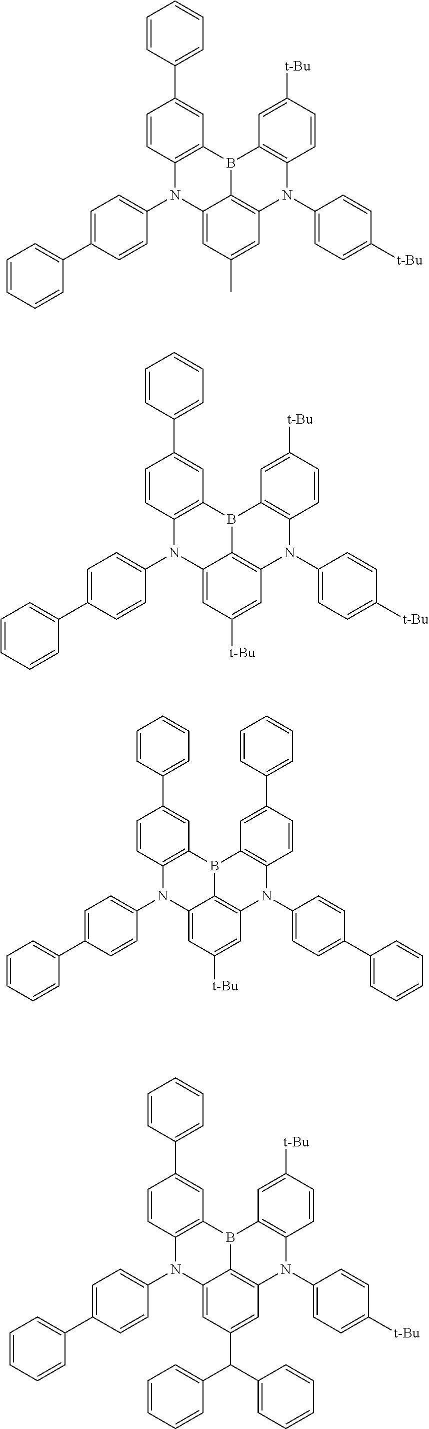 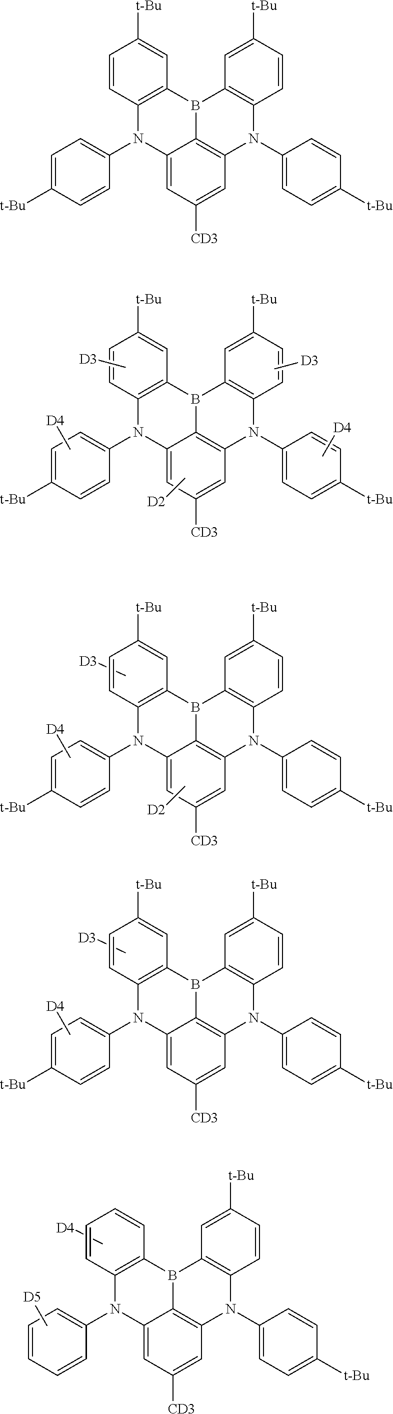 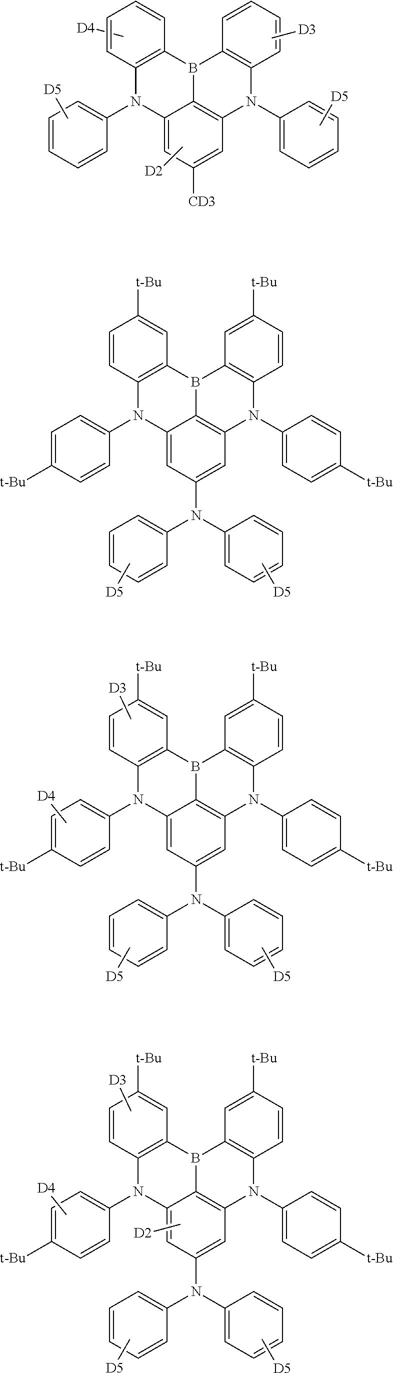 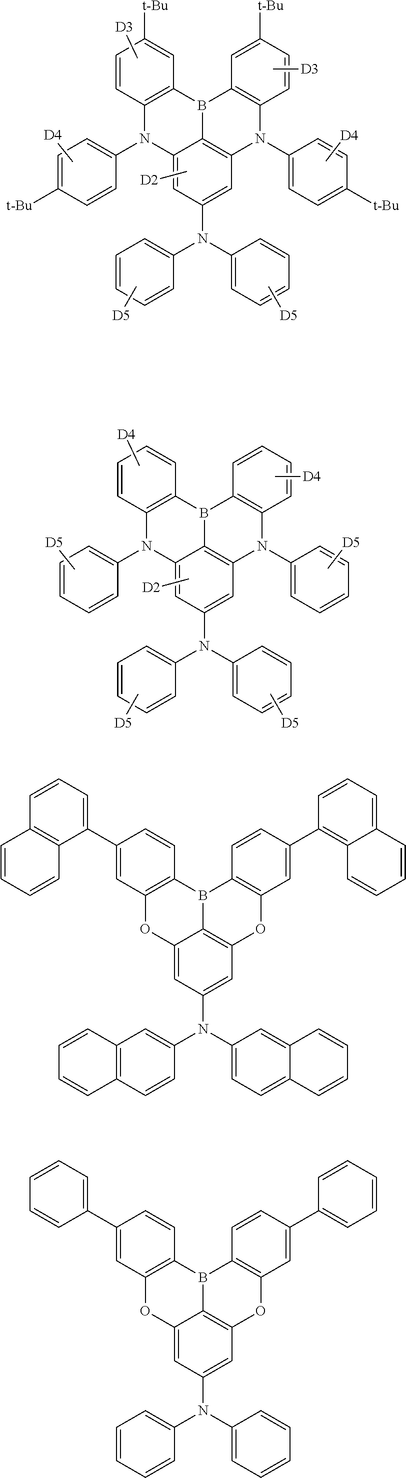 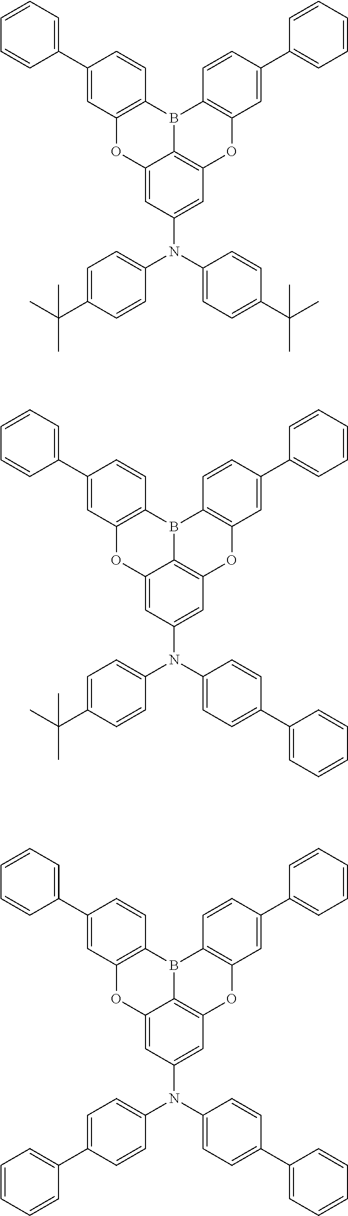 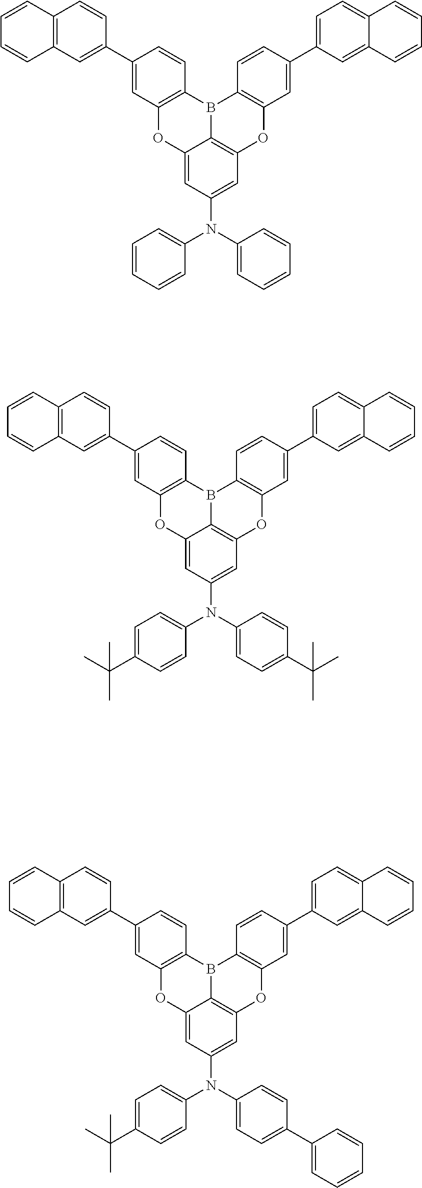 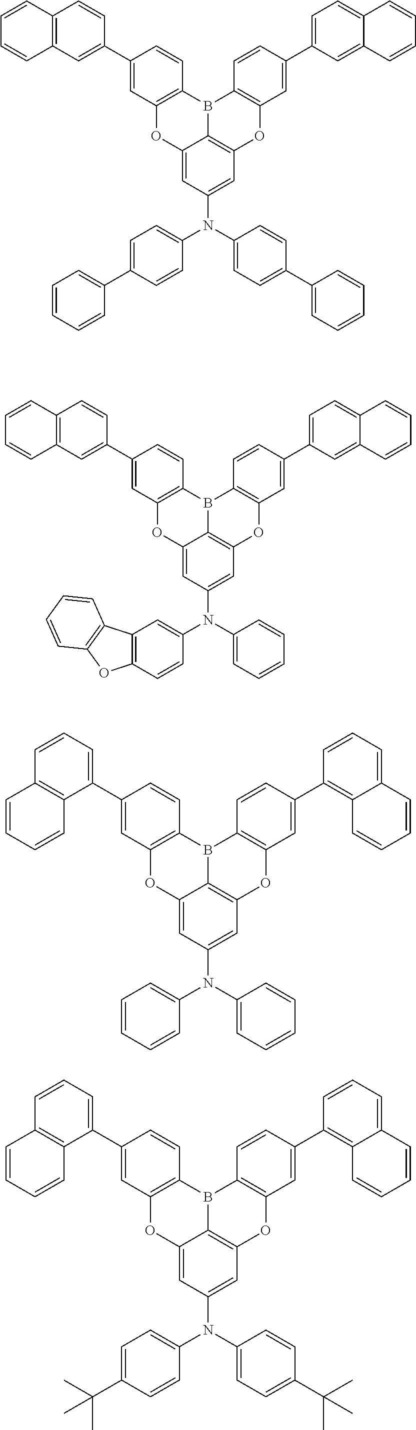 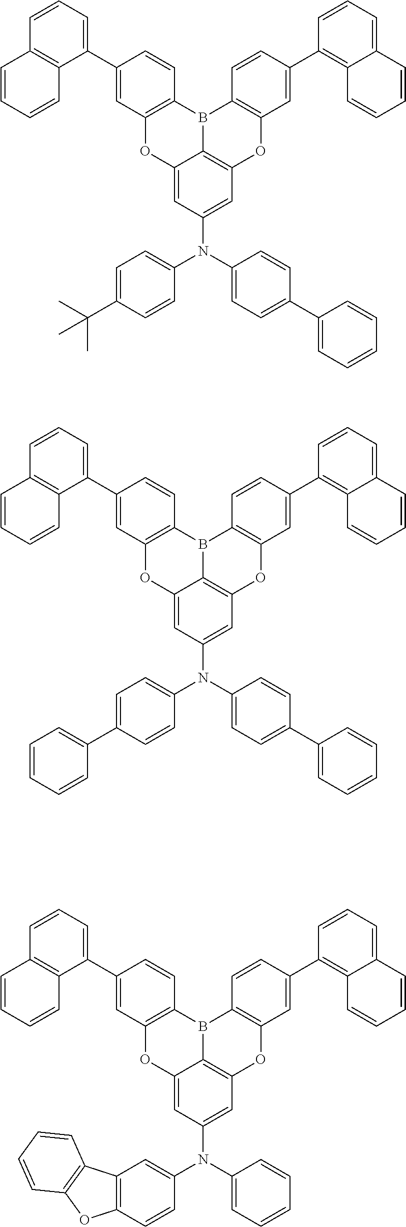 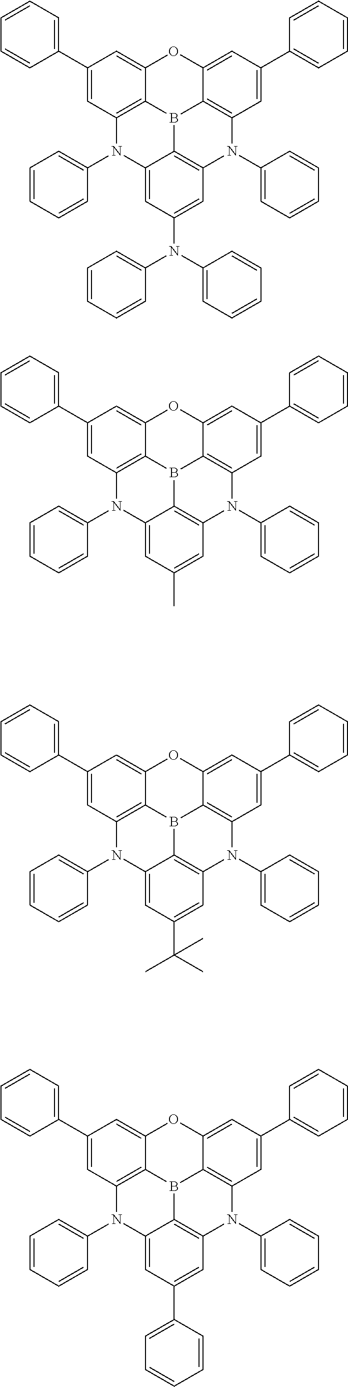 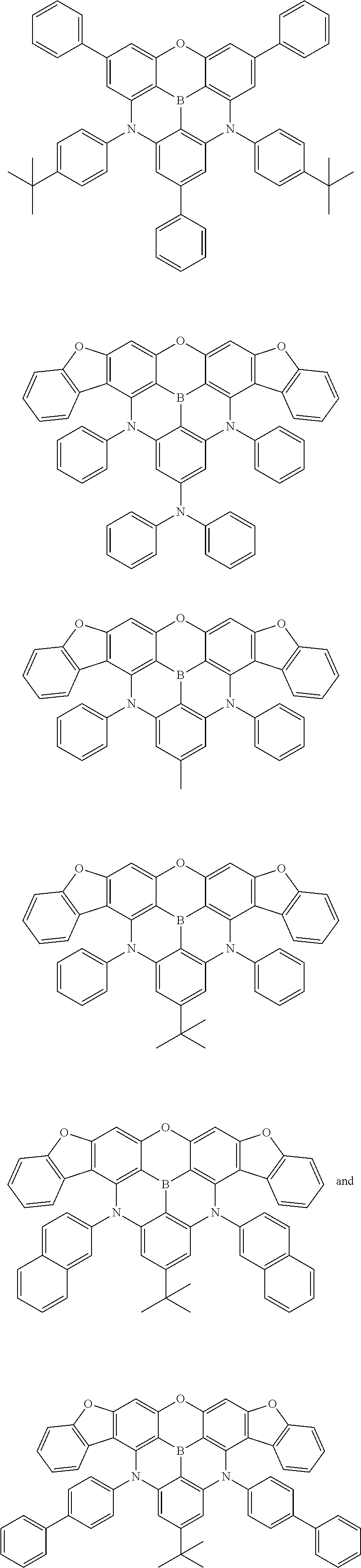
wherein, D2 to D5 respectively represent that 2 to 5 hydrogens are replaced with deuterium.

9. The plurality of organic electroluminescent materials according to claim 1, wherein the compound represented by formula 1 is comprised as a hole transport zone material, and the compound represented by formula 2 is comprised as a dopant material.

10. An organic electroluminescent device comprising a first electrode, a second electrode facing the first electrode, and an organic layer between the first and second electrodes, wherein the organic layer comprises the plurality of organic electroluminescent materials according to claim 1.

11. The organic electroluminescent device according to claim 10, wherein the organic layer comprises at least one layer of hole transport zone and at least one light-emitting layer, and at least one layer of the hole transport zone comprises the compound represented by formula 1, and at least one layer of the light-emitting layer comprises the compound represented by formula 2.
